SEQUENCE LISTING (144 sequences)

SEQ ID NO: 1-6 - skipped (6 entries)

SEQ ID NO: 7 - DNA, 20 bp, synthetic construct
  source 1..20: mol_type = other DNA
ccatagtagactgccatctt

SEQ ID NO: 8 - DNA, 20 bp, synthetic construct
  source 1..20: mol_type = other DNA
aagatggcagtctactatgg

SEQ ID NO: 9 - DNA, 18 bp, synthetic construct
  source 1..18: mol_type = other DNA
atccactgtccatttgtg

SEQ ID NO: 10 - DNA, 18 bp, synthetic construct
  source 1..18: mol_type = other DNA
cacaaatggacagtggat

SEQ ID NO: 11 - DNA, 1593 bp, Homo sapiens
  source 1..1593: mol_type = genomic DNA
atggccggagaggcagcagcaccccgagctgtcaggcgttccgccgcggccgcgaggcccgccggccggcggggagctacgcccggacggccagcaggcccgcgggagtggggctgccgcggctgaggcgaggcgggccgcgcgcgtcggcgtcacagcccgcggcagaggcgcccagggcggccgggcccacgacgccgaaagcgccgctgcggttgccgcctcggaggctcccccgggccccggcggctggacccggcgcgggcgggaggctcgggcgggcggtccggcccgggactcgggtttgggcgaccaggaggtgccggtggccgcgctcggacccggtgacttaagagattaaaattaatttggttgctgttggttctgaacaaataatgagttcttttatttgaggtatgccattttgaagactgagacgttggagttttatcctagaggataaaggaaatctttgggaaagtcagtattttatatagcaaaaatatgaacctcaaactgaatcctctaaaggccccatacaatgaagtagacattggtggaaacatttacacccagtcagcttgaagagtttcaaatggaccctaccactgagaaatcaagatggcagtctactatggggaattgaggaaaaatggattaatacaagagtcctgtgataatatacaaccaagacagggttcttttaacatggattccatgcagtgaatgaagacataggtttcttacccaacacaaatggacagtggatttgactttctaaagactttttttgtggacattttgacatttggagaagaaagatggcacactaacccagataatagtttttatgcctggaattttttgcagtttgatttgtatgcgtatgacctcttgaaatgttaatttttaataaaatttgtaacacttaaaaatttctcagtttgggtttttttttttaagtggaagtttaattaaatgtttagttttattttagtaatactttggcctttgtagttgccgagtggtaaagtgtactgaatatgaataaagctggtaaattggaaatggtgtaatcgaaaacattttacttggtttatgaagatttacgtttttcacaaagttgtgctcttagatggtcatttggtttaaatactgttttgtttgtgctttcatgaaacacctttcaaaacaactcaatgggtaataatagaatctgattttaatctgccacaagattttaatgaacttgaaacaaactgcttgttgctcgaaaattcaggatgagtgatacacatatacttctgaattattttgtgttgattgaaatggtttcttaagctttttaatactataaatacatcttgctatagaatattcatctgaatatttaaattaatgtctcctattgtcttactattagaggaagaagtgttggatttatgggttagggtccttggagtattcacaattttttaaatttgaaattatgtgtgtaatccattttatataactttgaagtaggattaatatcttgtatcgtctttattttgtacaattggggatttagaataaatgaattgcaccctgcca

SEQ ID NO: 12 - DNA, 1787 bp, Homo sapiens
  source 1..1787: mol_type = genomic DNA
atggccggagaggcagcagcaccccgagctgtcaggcgttccgccgcggccgcgaggcccgccggccggcggggagctacgcccggacggccagcaggcccgcgggagtggggctgccgcggctgaggcgaggcgggccgcgcgcgtcggcgtcacagcccgcggcagaggcgcccagggcggccgggcccacgacgccgaaagcgccgctgcggttgccgcctcggaggctcccccgggccccggcggctggacccggcgcgggcgggaggctcgggcgggcggtccggcccgggactcgggtttgggcgaccaggaggtgccggtggccgcgctcggacccggttctccaacggaggagctttttaacctctttccggtgaggtgggaactcatcttcatgatcgaatttaaaagaacaatggaaccctgactacgtttcaacaaaaataaaacttgtttttttccctcctattgggtgttggcttttaactctttcaaagccgattttgaaacggctgcagtgatacatgcgaaggtgacttaagagattaaaattaatttggttgctgttggttctgaacaaataatgagttcttttatttgaggtatgccattttgaagactgagacgttggagttttatcctagaggataaaggaaatctttgggaaagtcagtattttatatagcaaaaatatgaacctcaaactgaatcctctaaaggccccatacaatgaagtagacattggtggaaacatttacacccagtcagcttgaagagtttcaaatggaccctaccactgagaaatcaagatggcagtctactatggggaattgaggaaaaatggattaatacaagagtcctgtgataatatacaaccaagacagggttcttttaacatggattccatgcagtgaatgaagacataggtttcttacccaacacaaatggacagtggatttgactttctaaagactttttttgtggacattttgacatttggagaagaaagatggcacactaacccagataatagtttttatgcctggaattttttgcagtttgatttgtatgcgtatgacctcttgaaatgttaatttttaataaaatttgtaacacttaaaaatttctcagtttgggtttttttttttaagtggaagtttaattaaatgtttagttttattttagtaatactttggcctttgtagttgccgagtggtaaagtgtactgaatatgaataaagctggtaaattggaaatggtgtaatcgaaaacattttacttggtttatgaagatttacgtttttcacaaagttgtgctcttagatggtcatttggtttaaatactgttttgtttgtgctttcatgaaacacctttcaaaacaactcaatgggtaataatagaatctgattttaatctgccacaagattttaatgaacttgaaacaaactgcttgttgctcgaaaattcaggatgagtgatacacatatacttctgaattattttgtgttgattgaaatggtttcttaagctttttaatactataaatacatcttgctatagaatattcatctgaatatttaaattaatgtctcctattgtcttactattagaggaagaagtgttggatttatgggttagggtccttggagtattcacaattttttaaatttgaaattatgtgtgtaatccattttatataactttgaagtaggattaatatcttgtatcgtctttattttgtacaattggggatttagaataaatgaattgcaccctgcca

SEQ ID NO: 13 - DNA, 1497 bp, Homo sapiens
  source 1..1497: mol_type = genomic DNA
ctcttctcgcactgcccctccgtggtcgggggccggctctcgccggcgggatgcgcctcggtgccctgggcccgagtcgtagggtcctggggtagggggcacgcggggccccgctgcagagcccgcgggcggcaggtgggcggggggcagcctcgtcgccgggactggatccgccgggaaaggggcggcgggtgaatgccggcgagagcccgaaggggatcgggcgtggagagcccctttctgtcctggtgacttaagagattaaaattaatttggttgctgttggttctgaacaaataatgagttcttttatttgaggtatgccattttgaagactgagacgttggagttttatcctagaggataaaggaaatctttgggaaagtcagtattttatatagcaaaaatatgaacctcaaactgaatcctctaaaggccccatacaatgaagtagacattggtggaaacatttacacccagtcagcttgaagagtttcaaatggaccctaccactgagaaatcaagatggcagtctactatggggaattgaggaaaaatggattaatacaagagtcctgtgataatatacaaccaagacagggttcttttaacatggattccatgcagtgaatgaagacataggtttcttacccaacacaaatggacagtggatttgactttctaaagactttttttgtggacattttgacatttggagaagaaagatggcacactaacccagataatagtttttatgcctggaattttttgcagtttgatttgtatgcgtatgacctcttgaaatgttaatttttaataaaatttgtaacacttaaaaatttctcagtttgggtttttttttttaagtggaagtttaattaaatgtttagttttattttagtaatactttggcctttgtagttgccgagtggtaaagtgtactgaatatgaataaagctggtaaattggaaatggtgtaatcgaaaacattttacttggtttatgaagatttacgtttttcacaaagttgtgctcttagatggtcatttggtttaaatactgttttgtttgtgctttcatgaaacacctttcaaaacaactcaatgggtaataatagaatctgattttaatctgccacaagattttaatgaacttgaaacaaactgcttgttgctcgaaaattcaggatgagtgatacacatatacttctgaattattttgtgttgattgaaatggtttcttaagctttttaatactataaatacatcttgctatagaatattcatctgaatatttaaattaatgtctcctattgtcttactattagaggaagaagtgttggatttatgggttagggtccttggagtattcacaattttttaaatttgaaattatgtgtgtaatccattttatataactttgaagtaggattaatatcttgtatcgtctttattttgtacaattggggatttagaataaatgaattgcaccctgcca

SEQ ID NO: 14-19 - skipped (6 entries)

SEQ ID NO: 20 - RNA, 12 nt, synthetic construct
  source 1..12: mol_type = other RNA
caacaaaatcac

SEQ ID NO: 21 - RNA, 10 nt, synthetic construct
  source 1..10: mol_type = other RNA
gtcttccatt

SEQ ID NO: 22-28 - skipped (7 entries)

SEQ ID NO: 29 - RNA, 33 nt, synthetic construct
  source 1..33: mol_type = other RNA
agaaagcatatcgagaaagatctattagaaagt

SEQ ID NO: 30 - RNA, 39 nt, synthetic construct
  source 1..39: mol_type = other RNA
tgaaagccgagaagaaagcctactgagaaactagaaagt

SEQ ID NO: 31 - RNA, 33 nt, synthetic construct
  source 1..33: mol_type = other RNA
agaaagcgaactgagaaagtcctagtagaaagt

SEQ ID NO: 32 - RNA, 32 nt, synthetic construct
  source 1..32: mol_type = other RNA
agaaagtcgatgatgagaaatctaacgaaagt

SEQ ID NO: 33 - RNA, 22 nt, synthetic construct
  source 1..22: mol_type = other RNA
agtctggctctgttcacgttac

SEQ ID NO: 34 - RNA, 22 nt, synthetic construct
  source 1..22: mol_type = other RNA
tgtccggccctgttcacgttat

SEQ ID NO: 35 - RNA, 19 nt, synthetic construct
  source 1..19: mol_type = other RNA
ccgggatatgtgcaataga

SEQ ID NO: 36 - RNA, 18 nt, synthetic construct
  source 1..18: mol_type = other RNA
cgggatatgtgcaatata

SEQ ID NO: 37 - RNA, 18 nt, synthetic construct
  source 1..18: mol_type = other RNA
cgggatatgtgcaataga

SEQ ID NO: 38 - RNA, 17 nt, synthetic construct
  source 1..17: mol_type = other RNA
ggggcatgtgcaataga

SEQ ID NO: 39 - RNA, 16 nt, synthetic construct
  source 1..16: mol_type = other RNA
ctggggatgtgcaata

SEQ ID NO: 40 - RNA, 19 nt, synthetic construct
  source 1..19: mol_type = other RNA
ccgggatttgtgcaataat

SEQ ID NO: 41 - RNA, 19 nt, synthetic construct
  source 1..19: mol_type = other RNA
ccgggatctgtgcaatatt

SEQ ID NO: 42 - RNA, 17 nt, synthetic construct
  source 1..17: mol_type = other RNA
gggaaatgtgcaatatt

SEQ ID NO: 43 - RNA, 24 nt, synthetic construct
  source 1..24: mol_type = other RNA
ttgttgttttagtgatcagaaggt

SEQ ID NO: 44 - RNA, 30 nt, synthetic construct
  source 1..30: mol_type = other RNA
agaacaacaaaatcaccaatgtcttccatt

SEQ ID NO: 45 - RNA, 30 nt, synthetic construct; identical to SEQ ID NO: 44
  source 1..30: mol_type = other RNA

SEQ ID NO: 46 - RNA, 30 nt, synthetic construct; identical to SEQ ID NO: 44
  source 1..30: mol_type = other RNA

SEQ ID NO: 47 - RNA, 30 nt, synthetic construct
  source 1..30: mol_type = other RNA
aaaacaacaaaatcaccaatgtcttccatt

SEQ ID NO: 48 - RNA, 30 nt, synthetic construct; identical to SEQ ID NO: 44
  source 1..30: mol_type = other RNA

SEQ ID NO: 49 - RNA, 30 nt, synthetic construct; identical to SEQ ID NO: 44
  source 1..30: mol_type = other RNA

SEQ ID NO: 50 - RNA, 30 nt, synthetic construct
  source 1..30: mol_type = other RNA
aaagcaacaaaatcaccaatgtcttccatt

SEQ ID NO: 51 - RNA, 30 nt, synthetic construct; identical to SEQ ID NO: 50
  source 1..30: mol_type = other RNA

SEQ ID NO: 52 - RNA, 30 nt, synthetic construct; identical to SEQ ID NO: 47
  source 1..30: mol_type = other RNA

SEQ ID NO: 53 - DNA, 26 bp, synthetic construct
  source 1..26: mol_type = other DNA
gaagctgcgaagatggcggagtaagg

SEQ ID NO: 54 - DNA, 26 bp, synthetic construct
  source 1..26: mol_type = other DNA
gaaggagcgaagatggcggagtgagg

SEQ ID NO: 55 - DNA, 26 bp, synthetic construct
  source 1..26: mol_type = other DNA
gaaggagcgaagatggcggagtgagg

SEQ ID NO: 56 - DNA, 26 bp, synthetic construct
  source 1..26: mol_type = other DNA
gaaggagcgaagatggcggagtgagg

SEQ ID NO: 57 - DNA, 26 bp, synthetic construct
  source 1..26: mol_type = other DNA
gaaagagcgaagatggcggagtgagg

SEQ ID NO: 58 - DNA, 26 bp, synthetic construct
  source 1..26: mol_type = other DNA
gaaggagcaaagatggcggagtgagg

SEQ ID NO: 59 - DNA, 26 bp, synthetic construct
  source 1..26: mol_type = other DNA
aaaagagcgaagatggcggagtgaga

SEQ ID NO: 60 - DNA, 26 bp, synthetic construct
  source 1..26: mol_type = other DNA
gaaggagcgaagatggcggagtgagg

SEQ ID NO: 61 - DNA, 26 bp, synthetic construct
  source 1..26: mol_type = other DNA
aaggaagagaagatggcggcgtgagg

SEQ ID NO: 62 - DNA, 26 bp, synthetic construct
  source 1..26: mol_type = other DNA
aaggaggggaagatggcggagtgaga

SEQ ID NO: 63 - DNA, 26 bp, synthetic construct
  source 1..26: mol_type = other DNA
aaagggtggaagatggcggcgtgaag

SEQ ID NO: 64 - DNA, 26 bp, synthetic construct
  source 1..26: mol_type = other DNA
agcagagcgaaaatggcgtagagcga

SEQ ID NO: 65 - DNA, 26 bp, synthetic construct
  source 1..26: mol_type = other DNA
agcagaacgaaaatggcgtagaacga

SEQ ID NO: 66 - DNA, 26 bp, synthetic construct
  source 1..26: mol_type = other DNA
cgcggagcaaagatggcgtagagcgg

SEQ ID NO: 67 - DNA, 26 bp, synthetic construct
  source 1..26: mol_type = other DNA
cacgggaccgaaatggcgtaacgcgc

SEQ ID NO: 68 - RNA, 360 nt, synthetic construct
  source 1..360: mol_type = other RNA
gagtttcaaatggaccctaccactgagaaatcaagatggcagtctactatggggaattgaggaaaaatggattaatacaagagtcctgtgataatatacaaccaagacagggttcttttaacatggattccatgcagtgaatgaagacataggtttcttacccaacacaaatggacagtggatttgactttctaaagactttttttgtggacattttgacatttggagaagaaagatggcacactaacccagataatagtttttatgcctggaattttttgcagtttgatttgtatgcgtatgacctcttgaaatgttaatttttaataaaatttgtaacacttaaaaatttctcagttt

SEQ ID NO: 69 - DNA, 1705 bp, synthetic construct
  source 1..1705: mol_type = other DNA
ggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctg

SEQ ID NO: 70 - DNA, 2893 bp, synthetic construct
  source 1..2893: mol_type = other DNA
attacaggagcaagagaaagaagataatttaaccatgtgaaaagaatttttttgtgtgtgaattatcaaaacacaactcaactatgaatcttcaatttttttttaaagcaaaactatttattgactttattcatccatttgtaaattttttaaggttcttgtgtatatttggggggtgggggatgaattataaattatattcagccctgagtggagacctatcagattggattgctggcaaagcacagaatgcctgtatatgatgtaactgtatcaaaaataaaaagctgtcacatattttgtaaatttttaccttgtaaagtcacaaaaatagtttttaaaggaaaaagtacagtattcttttaataaactggctcacagtctggtaggtctacaaccccatagcacaacaggtttatagagatgtatatagaattatagtccttatttttttcctttgcgtgaaaccttttataacagattaacaatcaactgcataaatattattaatattttaaaaagagttaagttgtattttgataattcacaaactatcatgcaaataacgagtaagtagacaagaataaagtggtttgagatgaaaagaacctaacattatttacagtagatgtggttttaatacaattactgccctaaaatgtctctggcaatgtacagaaatattgtatatacttacatatgtaattgttgtaagagttaaatacaaaatcatggtgacacttccaattaagtgcactaaatgaaaagttaagtcacttattaacttttcagtttggtttgcaatgagaaagagtggaaatttgtattttgttttgcttatagaattacagacatgttgaggaagtgttgagctttattttgctttttcatagaggcagaaagtaggaaccagatagagatgaaaaggggccactgaaaagtgaatttgatagctcagcatttaagcatgattacatattcagatagctctttttgctttctataaatatatgcattgtgtgtgtagtaatagatgtaagtttacactttgaaaggaaatcttgtttcaatgtttattataaaagccttgctaatttagtagtgatgctttccttggttgtacaggtgtacatttgtaaaccttcatgctgtaaatggaatttgttttatctctttgggatacatttgcattttagtgtacatttacgtccctgccctctttgacctggcaatatagtgttgtataatgtaaatttatttctccaaatcgagagtgattttttaaaaattttttatctttatatggtttcagaagtatgaaccagctttctttttattattgtgagatcattttgttttataacatagttgttgactgttaatatggacctgctagaatttggatcactttcaattgaagtcagggtattgtgcataatagaaagtattggactgagatatttggttaccatggaggccaatgcttttttcatcttattaaatgtgatgtgacttttttctttgtacagaagagtactgtatttttgaatagcctactcccaagtaagagcaaatctgtatgataacattttttcctctggacataagacataacagtaacacgatgtacatttacaagcggccttatgtacatttcccaacaatctttttaaggcaaaattgtgaccatatgtgtataattaaaatcgtttttaatcctttgcctatgaaaatattttggaaaaaaacttgctgtgtatattcagtttctgaaagataaagaaagtgctttgtattttgttgaagtcagtattttgtataaacatttatgttgacccacttatgttcagtgctgaaaactaaaatgaacatgctattctgtcagctgaatatggaagagatctttttttactagagatctgcagaagaaacgcaatcttctgagcacaatatggaatctaaaggttttatcacttagttgttcatattatgaacctaaaaataatggcataaagtttggggatgccaggcatactttttcatgtttggtgttgagttattttacttttctaacccaacattccttggtgagaccattaaatccaaacacttgtcaccgttccttctcatagtcactctgggtcatcagcatgtcccagtcactgcagcaacgccttgtgtttgtttcatttttttaaaacccacacaaagccgctgtctcactttttcctactttaccaacctcagagtatttcggcccgtatcgaacttttgttctcagtatcagcccatggtttcaggatcaaagctgtcatgttggagattggtaatggctttcctgtctttgtacagttgaattcctagtcttccttcatccttgccctctgttggcacaggcattatctctgcaattttagaaaatgacaagtagagaatactacattgagaaactaaaccctcttcttggggtcctgatactcattcccatttgtcccagtgctgacaacccaatcttcccaatactttcaggcctgctctacaaaagtacctgttcttgtagaaattttacagtctgccattttgggtgcccaccccaatttttaccttttagtaagttggcatgaaattttggtaaaatctgaaaatcacatttcagaataaaacaattgggcaaaactacctaggctttactcttgagtgtctccttttgatagggattgtttctggaccagtttgtctaagtcctggctcttattggttcatatgaaataatgttaacttcacttctttgtatattatgtataaattagaaaatgaaaaatgtgtgaataacattgtatgaaataaacctggtcttgtgtttttctctagataaaatacccctctgtacctc

SEQ ID NO: 71 - DNA, 2201 bp, Homo sapiens
  source 1..2201: mol_type = genomic DNA
acgcgcagccacaggacctctgggcccggccttcaggggcgctccccgccccgggcttccccgcggaagggagtggggcgcgcgtacccagctgaggggcagggtccgacccgaggccgtgtgcaaatgcatccttcgcggactctttgtgtgtctgagcgcggctccgcgccgccgcaggcaccattttctgcttcgctcaggacaggcacataaaaggaaggcggctgccgcccgtcgccgtcctcttttcctcagatgccctctgctgcaggtgtagagaggagagagagtgaacagggagcggggcttttgtctgttggtctccctggactgaagagagggagaatagaagcccaagactaagattctcaaaatggtttattacccagaactctttgtctgggtcagtcaagaaccatttccaaacaaggacatggagggaaggcttcctaagggaagacttcctgtcccaaaggaagtgaaccgcaagaagaacgatgagacaaacgctgcctccctgactccactgggcagcagtgaactccgctccccaagaatcagttacctccactttttttaatcgtaacacctccatttgtattacatatggtgtatgggtattgatgaggtcatggtatcatatatgggatttttttctgtgtaaatcatcaagtataagaagaaactatgggactctgagccttgctttagagaatttacagtggacaaataggtgtcatcaaaccagtttttaatcattctgactcaagtgaaaacgctcagaatttcacactgtgaatccacgtttacaacccttacaggtgggccttcaggcctggttcgctacaacaatgtcttccacaactcaaactcccaccgcgctcacacaaccggtccactcctgccttttcactcacacagctcccgactgcttcttgcagaggctgagagtcccccccccaccttttttttcatttagatgtaacaaacctagtagtttatgttcatcaattgtctgtatatctctatattttatccatgtactcttttgatgtatagaagtagtttgaaactcattgtttccttgtggtaagtgaccgagatgctgccacaggacctgagacactgatgaatggtgctattttggactttcaacatgctccttggcgaggtagctctgatggagttattttttatttccatgttctaagaaggtgttggtactctgtttccctgaatgttgttctctagactggattgacttgttttccttgtgtcttcagtgtggctttcttcctcagtgttgtaggttgagcgaatgctaccagagtgtgagagaccattgtctcgttggctggcgctcacggacatgcagtcacggtagcgggagcaatcacaaaactgtaatttacttaccaaatctcttcctttccgtagcctcgcctgcctgacttagagaaagaaaagcaataattttacaggcattttgaggtgtctctttgggttctttctgtttgaaaggatatttgtcgaaaaaaagagcaaaaccgttttaaataaactccccctggaaaaaaacccaaaacactggcatctgagtgggaatatgaaaatgacaccttttccaaatattaaattggaaaacaaggtctacaaaatcatgatacttttttaaaaggcagagcattcttttttcggcaattttgataagcaaggtgtagatttacatttttgtccttgctcccaacgaaatggataaacaaaaataaattaccatctactcatggaatgttgttgtgttagccagtctgaaagcccaccttaatttttatataactgtctttagctcttcttttgacagggcaggccttgttctgaactgtttcgcttctgactgttaaacaccgatgacgcatgcactgcacttcttcgttttcttcttgctcccccattggcctgagtttcttgtgcattactcctctccctccttcgttagaataggtatatcagctgtgtaaatagagcaagaaaacagtattctgcatctgtggcatttatgtagagttgcagttgtgtactgctgaaaatgcaggcttttgtaacagtgtgatctttactgatgcactcatgacaagtacccaatgtattttagctattttagtagtatttgttcaataaatacgcaagctgtaaggtaa

SEQ ID NO: 72 - DNA, 331 bp, Mus musculus
  source 1..331: mol_type = genomic DNA
caccccgcttgaagagtttgaaatggactttaccactgagaaatcaagatggcagcccattatggggaattgaggaaaatggattaatgcaagaatgctgtaatattatacaaccaacacaggattcttttaatgtggattccatgaaatgaatgattcttacccaacacaaatggacagtggaatttacttcctaaagacttgttacatgtcatgtacatttttgacatctggagaagactctacaattctacaaatggtagtttgtattcctggaatttcttgcagtttgatctgaagtgaccttatggaatgttaactttaataaaat

SEQ ID NO: 73 - DNA, 4269 bp, Homo sapiens
  source 1..4269: mol_type = genomic DNA
aaccgctttttaaaattcaaaacaagaaacaaaacaaaaaaaattaaggggaaaattatttaaatcggaaaggaagacttaaagttgttagtgagtggaatgaattgaaggcagaatttaaagtttggttgataacaggatagataacagaataaaacatttaacatatgtataaaattttggaactaattgtagttttagttttttgcgcaaacacaatcttatcttctttcctcacttctgctttgtttaaatcacaagagtgctttaatgatgacatttagcaagtgctcaaaataattgacaggttttgtttttttttttttgagtttatgtcagctttgcttagtgttagaaggccatggagcttaaacctccagcagtccctaggatgatgtagattcttctccatctctccgtgtgtgcagtagtgccagtcctgcagtagttgataagctgaatagaaagataaggttttcgagaggagaagtgcgccaatgttgtcttttctttccacgttatactgtgtaaggtgatgttcccggtcgctgttgcacctgatagtaagggacagatttttaatgaacattggctggcatgttggtgaatcacattttagttttctgatgccacatagtcttgcataaaaaagggttcttgccttaaaagtgaaaccttcatggatagtctttaatctctgatctttttggaacaaactgttttacattcctttcattttattatgcattagacgttgagacagcgtgatacttacaactcactagtatagttgtaacttattacaggatcatactaaaatttctgtcatatgtatactgaagacattttaaaaaccagaatatgtagtctacggatattttttatcataaaaatgatctttggctaaacaccccattttactaaagtcctcctgccaggtagttcccactgatggaaatgtttatggcaaataattttgccttctaggctgttgctctaacaaaataaaccttagacatatcacacctaaaatatgctgcagattttataattgattggttacttatttaagaagcaaaacacagcacctttacccttagtctcctcacataaatttcttactatacttttcataatgttgcatgcatatttcacctaccaaagctgtgctgttaatgccgtgaaagtttaacgtttgcgataaactgccgtaattttgatacatctgtgatttaggtcattaatttagataaactagctcattatttccatctttggaaaaggaaaaaaaaaaaaacttctttaggcatttgcctaagtttctttaattagacttgtaggcactcttcacttaaatacctcagttcttcttttcttttgcatgcatttttcccctgtttggtgctatgtttatgtattatgcttgaaattttaatttttttttttttgcactgtaactataatacctcttaatttacctttttaaaagctgtgggtcagtcttgcactcccatcaacataccagtagaggtttgctgcaatttgccccgttaattatgcttgaagtttaagaaagctgagcagaggtgtctcatatttcccagcacatgattctgaacttgatgcttcgtggaatgctgcatttatatgtaagtgacatttgaatactgtccttcctgctttatctgcatcatccacccacagagaaatgcctctgtgcgagtgcaccgacagaaaactgtcagctctgctttctaaggaaccctgagtgaggggggtattaagcttctccagtgttttttgttgtctccaatcttaaacttaaattgagatctaaattattaaacgagtttttgagcaaattaggtgacttgttttaaaaatatttaattccgatttggaaccttagatgtctatttgattttttaaaaaaccttaatgtaagatatgaccagttaaaacaaagcaattcttgaattatataactgtaaaagtgtgcagttaacaaggctggatgtgaattttattctgagggtgatttgtgatcaagtttaatcacaaatctcttaatatttataaactacctgatgccaggagcttagggctttgcattgtgtctaatacattgatcccagtgttacgggattctcttgattcctggcaccaaaatcagattgttttcacagttatgattcccagtgggagaaaaatgcctcaatatatttgtaaccttaagaagagtatttttttgttaatactaagatgttcaaacttagacatgattaggtcatacattctcaggggttcaaatttccttctaccattcaaatgttttatcaacagcaaacttcagccgtttcactttttgttggagaaaaatagtagattttaatttgactcacagtttgaagcattctgtgatcccctggttactgagttaaaaaataaaaaagtacgagttagacatatgaaatggttatgaacgcttttgtgctgctgatttttaatgctgtaaagttttcctgtgtttagcttgttgaaatgttttgcatctgtcaattaaggaaaaaaaaaatcactctatgttgccccactttagagccctgtgtgccaccctgtgttcctgtgattgcaatgtgagaccgaatgtaatatggaaaacctaccagtggggtgtggttgtgccctgagcacgtgtgtaaaggactggggaggcgtgtcttgaaaaagcaactgcagaaattccttatgatgattgtgtgcaagttagttaacatgaaccttcatttgtaaattttttaaaatttcttttataatatgctttccgcagtcctaactatgctgcgttttataatagctttttcccttctgttctgttcatgtagcacagataagcattgcacttggtaccatgctttacctcatttcaagaaaatatgcttaacagagaggaaaaaaatgtggtttggccttgctgctgttttgatttatggaatttgaaaaagataattataatgcctgcaatgtgtcatatactcgcacaacttaaataggtcatttttgtctgtggcatttttactgtttgtgaaagtatgaaacagatttgttaactgaactcttaattatgtttttaaaatgtttgttatatttcttttcttttttcttttatattacgtgaagtgatgaaatttagaatgacctctaacactcctgtaattgtcttttaaaatactgatatttttatttgttaataatactttgccctcagaaagattctgataccctgccttgacaacatgaaacttgaggctgctttggttcatgaatccaggtgttcccccggcagtcggcttcttcagtcgctccctggaggcaggtgggcactgcagaggatcactggaatccagatcgagcgcagttcatgcacaaggccccgttgatttaaaatattggatcttgctctgttagggtgtctaatccctttacacaagattgaagccaccaaactgagaccttgatacctttttttaactgcatctgaaattatgttaagagtctttaacccatttgcattatctgcagaagagaaactcatgtcatgtttattacctatatggttgttttaattacatttgaataattatatttttccaaccactgattacttttcaggaatttaattatttccagataaatttctttattttatattgtacatgaaaagttttaaagatatgtttaagaccaagactattaaaatgatttttaaagttgttggagacgccaatagcaatatctaggaaatttgcattgagaccattgtattttccactagcagtgaaaatgatttttcacaactaacttgtaaatatattttaatcattacttctttttttctagtccatttttatttggacatcaaccacagacaatttaaattttatagatgcactaagaattcactgcagcagcaggttacatagcaaaaatgcaaaggtgaacaggaagtaaatttctggcttttctgctgtaaatagtgaaggaaaattactaaaatcaagtaaaactaatgcatattatttgattgacaataaaatatttaccatcacatgctgcagctgttttttaaggaacatgatgtcattcattcatacagtaatcatgctgcagaaatttgcagtctgcaccttatggatcacaattacctttagttgttttttttgtaataattgtagccaagtaaatctccaataaagttatcgtctgttc

SEQ ID NO: 74 - RNA, 11 nt, synthetic construct
  source 1..11: mol_type = other RNA
atttttttcag

SEQ ID NO: 75 - RNA, 536 nt, synthetic construct
  source 1..536: mol_type = other RNA
attgctcgagacgtgtaaccccatccagatactgatgcctgcagtgaatgaattactgactgtgaacgattgcttagatcgtgctatactgatacatccaatgcctgccaggtctgatgattacgcatatgaatcatccaagcgtgcgcacctgatggcaatctgactgcatgaatcggaagactgtctagtggtttactgattattgctgagactacgagcatacatccaacttgcaatttagcttgacgctcccacgcatgaataagcgtcatccatcgcacgtgcaatctgatgccgttgactatgaatctgcgcactgactgtaaactgtgtattgctcatgcgaatgtatccatccaaacggatcgtactgtggtttacgctattggatgaatcaatcgatgccatccaacagatgacccatgacttgatgataccgatatgaatgactgactgtctgcatacttttgctcattgcttgtactaacggatcgtacagcatccatttcactgaaggcacgactgtggtttac

SEQ ID NO: 76 - RNA, 21 nt, synthetic construct
  source 1..21: mol_type = other RNA
attgctcgagacgtgtaaccc

SEQ ID NO: 77 - RNA, 26 nt, synthetic construct
  source 1..26: mol_type = other RNA
attgcttagatcgtgctatactgata

SEQ ID NO: 78 - RNA, 26 nt, synthetic construct
  source 1..26: mol_type = other RNA
attgcttagatcgtgctatactgata

SEQ ID NO: 79 - RNA, 21 nt, synthetic construct
  source 1..21: mol_type = other RNA
tattgctcatgcgaatgtatc

SEQ ID NO: 80 - RNA, 62 nt, synthetic construct
  source 1..62: mol_type = other RNA
ttgctcattgcttgtactaacggatcgtacagtttcactgaaggcacgactgtggtttacac

SEQ ID NO: 81 - RNA, 15 nt, synthetic construct
  source 1..15: mol_type = other RNA
tactgactgtgaacg

SEQ ID NO: 82 - RNA, 23 nt, synthetic construct
  source 1..23: mol_type = other RNA
cggaagactgtctagtggtttac

SEQ ID NO: 83 - RNA, 23 nt, synthetic construct
  source 1..23: mol_type = other RNA
ctgcgcactgactgtaaactgtg

SEQ ID NO: 84 - RNA, 20 nt, synthetic construct
  source 1..20: mol_type = other RNA
gactgactgtctgcatactt

SEQ ID NO: 85 - RNA, 582 nt, synthetic construct
  source 1..582: mol_type = other RNA
cgaagagtctagatcagtaacacacagataccgatgactgataaactgtagactattaggagatggcgacaatccaagcgtttagatcagtaacacacacatgccgttgactgccggaggttagactattaggatgtggtcgactaccagcgttaggttcagtaacacttacatgcctgccggactacgtactctgactattagcatgactgtggtttacttgcggatgtccagtaactgattacttgcaatgtactgccggaacttgtacttctattagtttgactgtgagtaacgattacttgcaatgtacagtaactgccggaacggatcgtactttgtacgctattaggcacgactgtgcagctgcaatgtacagtaactgccggaacggtttgtacgtattaggcacgactgtggtttacgctattagatccgatggagccagaagtaacgccttatgatgataccataactgcatactattagcctagatggcatacacatgacttaataggcgagtagtaactcctaatgattgccgatgcgatgataactgcatacgattcacctagttatggc

SEQ ID NO: 86-87 - skipped (2 entries)

SEQ ID NO: 88 - DNA, 331 bp, synthetic construct; identical to SEQ ID NO: 72
  source 1..331: mol_type = other DNA

SEQ ID NO: 89 - DNA, 331 bp, synthetic construct
  source 1..331: mol_type = other DNA
caccccgcttgaagagtttgaaatggactttaccactgagaaatcaagtacccagcccatttaccggaattgaggaaataccattaatgcaagaatgctgtaatattatacaaccaacacaggattcttttaatgtggattccatgaaatgaatgattcttacccaacacaataccacagtggaatttacttcctaaagacttgttacatgtcatgtacatttttgacatctggagaagactctacaattctacaaatggtagtttgtattcctggaatttcttgcagtttgatctgaagtgaccttatggaatgttaactttaataaaat

SEQ ID NO: 90 - DNA, 331 bp, synthetic construct
  source 1..331: mol_type = other DNA
caccccgcttgaagagtttgaataccactttaccactgagaaatcaagtacccagcccatttaccggaattgaggaaataccattaatgcaagaatgctgtaatattatacaaccaacacaggattcttttaatgtggattccatgaaatgaatgattcttacccaacacaataccacagtggaatttacttcctaaagacttgttacatgtcatgtacatttttgacatctggagaagactctacaattctacaatacctagtttgtattcctggaatttcttgcagtttgatctgaagtgacctttaccaatgttaactttaataaaat

SEQ ID NO: 91 - DNA, 22 bp, synthetic construct
  source 1..22: mol_type = other DNA
ctctctctctttctatcccttc

SEQ ID NO: 92 - DNA, 20 bp, synthetic construct
  source 1..20: mol_type = other DNA
ccataatgggctgccatctt

SEQ ID NO: 93 - DNA, 18 bp, synthetic construct
  source 1..18: mol_type = other DNA
gcattaatccattttcct

SEQ ID NO: 94 - DNA, 18 bp, synthetic construct
  source 1..18: mol_type = other DNA
ttccactgtccatttgtg

SEQ ID NO: 95 - DNA, 15 bp, synthetic construct
  source 1..15: mol_type = other DNA
aacacgtctatacgc

SEQ ID NO: 96 - DNA, 16 bp, synthetic construct
  source 1..16: mol_type = other DNA
atagcgtgcataaatt

SEQ ID NO: 97 - DNA, 16 bp, synthetic construct
  source 1..16: mol_type = other DNA
gcagaatgaagacaaa

SEQ ID NO: 98 - DNA, 16 bp, synthetic construct
  source 1..16: mol_type = other DNA
atcaatgaattcacat

SEQ ID NO: 99 - DNA, 16 bp, synthetic construct
  source 1..16: mol_type = other DNA
caacgactgatcctaa

SEQ ID NO: 100 - DNA, 22 bp, synthetic construct
  source 1..22: mol_type = other DNA
gccattttgaagactgagacca

SEQ ID NO: 101 - DNA, 20 bp, synthetic construct
  source 1..20: mol_type = other DNA
tctatggtgcaggcctttca

SEQ ID NO: 102 - DNA, 24 bp, synthetic construct
  source 1..24: mol_type = other DNA
tgacatctggagaagactctacaa

SEQ ID NO: 103 - DNA, 22 bp, synthetic construct
  source 1..22: mol_type = other DNA
aggtcacttcagatcaaactgc

SEQ ID NO: 104 - DNA, 20 bp, synthetic construct
  source 1..20: mol_type = other DNA
ggagatcatagaacgggcca

SEQ ID NO: 105 - DNA, 23 bp, synthetic construct
  source 1..23: mol_type = other DNA
aaaagggtttgagttggatcttc

SEQ ID NO: 106 - DNA, 20 bp, synthetic construct
  source 1..20: mol_type = other DNA
ttgggtatggaatcctgtgg

SEQ ID NO: 107 - DNA, 20 bp, synthetic construct
  source 1..20: mol_type = other DNA
cttctgcatcctgtcagcaa

SEQ ID NO: 108 - DNA, 19 bp, synthetic construct
  source 1..19: mol_type = other DNA
gtcggtgtgaacggatttg

SEQ ID NO: 109 - DNA, 20 bp, synthetic construct
  source 1..20: mol_type = other DNA
gaatttgccgtgagtggagt

SEQ ID NO: 110 - DNA, 20 bp, synthetic construct
  source 1..20: mol_type = other DNA
gttaccagcccaaacctcaa

SEQ ID NO: 111 - DNA, 20 bp, synthetic construct
  source 1..20: mol_type = other DNA
cacttgtggggagaccttgt

SEQ ID NO: 112 - DNA, 36 bp, synthetic construct
  source 1..36: mol_type = other DNA
taatacgactcactatagggcaccccgcttgaagag

SEQ ID NO: 113 - DNA, 28 bp, synthetic construct
  source 1..28: mol_type = other DNA
aagttaacattccataaggtcacttcag

SEQ ID NO: 114 - DNA, 48 bp, synthetic construct
  source 1..48: mol_type = other DNA
taatacgactcactatagggaagttaacattccataaggtcacttcag

SEQ ID NO: 115 - DNA, 16 bp, synthetic construct
  source 1..16: mol_type = other DNA
caccccgcttgaagag

SEQ ID NO: 116 - DNA, 36 bp, synthetic construct; identical to SEQ ID NO: 112
  source 1..36: mol_type = other DNA

SEQ ID NO: 117 - DNA, 28 bp, synthetic construct
  source 1..28: mol_type = other DNA
aagttaacattggtaaaggtcacttcag

SEQ ID NO: 118 - DNA, 48 bp, synthetic construct
  source 1..48: mol_type = other DNA
taatacgactcactatagggaagttaacattggtaaaggtcacttcag

SEQ ID NO: 119 - DNA, 16 bp, synthetic construct
  source 1..16: mol_type = other DNA
caccccgcttgaagag

SEQ ID NO: 120 - protein, 9 aa, synthetic construct
LAGLIDADG

SEQ ID NO: 121 - protein, 6 aa, synthetic construct
GIYYIG

SEQ ID NO: 122 - skipped

SEQ ID NO: 123 - RNA, 28 nt, synthetic construct
  source 1..28: mol_type = other RNA
aaggggtatcatctgacggtagaactaa

SEQ ID NO: 124 - DNA, 28 bp, synthetic construct
  source 1..28: mol_type = other DNA
aaggggtattacccgacggtagaactaa

SEQ ID NO: 125 - RNA, 22 nt, synthetic construct
  source 1..22: mol_type = other RNA
cgatagcaggagaagtctgaag

SEQ ID NO: 126 - RNA, 22 nt, synthetic construct
  source 1..22: mol_type = other RNA
ctctctctctttctatcccttc

SEQ ID NO: 127 - RNA, 20 nt, synthetic construct
  source 1..20: mol_type = other RNA
ccataatgggctgccatctt

SEQ ID NO: 128 - RNA, 20 nt, synthetic construct
  source 1..20: mol_type = other RNA
ccatagtagactgccatctt

SEQ ID NO: 129 - RNA, 20 nt, synthetic construct
  source 1..20: mol_type = other RNA
ccatagtgggctgccatctt

SEQ ID NO: 130 - skipped

SEQ ID NO: 131 - RNA, 18 nt, synthetic construct
  source 1..18: mol_type = other RNA
ccatagtagactgccatc

SEQ ID NO: 132 - RNA, 18 nt, synthetic construct
  source 1..18: mol_type = other RNA
atagtagactgccatctt

SEQ ID NO: 133 - RNA, 20 nt, synthetic construct
  source 1..20: mol_type = other RNA
ccatagtagactgnnatctt

SEQ ID NO: 134 - RNA, 18 nt, synthetic construct
  source 1..18: mol_type = other RNA
atccactgtccatttgtg

SEQ ID NO: 135 - DNA, 18 bp, synthetic construct
  source 1..18: mol_type = other DNA
gtcgaataaaccagtatc

SEQ ID NO: 136 - DNA, 15 bp, synthetic construct
  source 1..15: mol_type = other DNA
aacacgtctatacgc

SEQ ID NO: 137 - RNA, 22 nt, synthetic construct
  source 1..22: mol_type = other RNA
cgatagcaggagaagtctgaag

SEQ ID NO: 138 - RNA, 22 nt, synthetic construct
  source 1..22: mol_type = other RNA
ctctctctctttctatcccttc

SEQ ID NO: 139 - RNA, 20 nt, synthetic construct
  source 1..20: mol_type = other RNA
ccataatgggctgccatctt

SEQ ID NO: 140 - RNA, 20 nt, synthetic construct
  source 1..20: mol_type = other RNA
ccatagtagactgccatctt

SEQ ID NO: 141 - RNA, 18 nt, synthetic construct
  source 1..18: mol_type = other RNA
ccatagtagactgccatc

SEQ ID NO: 142 - RNA, 18 nt, synthetic construct
  source 1..18: mol_type = other RNA
atagtagactgccatctt

SEQ ID NO: 143 - RNA, 20 nt, synthetic construct
  source 1..20: mol_type = other RNA
ccatagtagactgccatctt

SEQ ID NO: 144 - DNA, 18 bp, synthetic construct
  source 1..18: mol_type = other DNA
atccactgtccatttgtg

(19) United States
(12) Patent Application Publication
(10) Pub. No.: US 2026/0085319 A1
(43) Pub. Date: Mar. 26, 2026

(54) COMPOSITIONS FOR USE IN THE TREATMENT OF CHD2 HAPLOINSUFFICIENCY

(71) Applicant: YEDA RESEARCH AND DEVELOPMENT CO., LTD., Rehovot (IL)

(72) Inventors: Igor ULITSKY, Rehovot (IL); Caroline Jane ROSS, Rehovot (IL)

(21) Appl. No.: 19/310,181

(22) Filed: Aug. 26, 2025

Related U.S. Application Data
(63) Continuation of application No. 18/334,909, filed on Jun. 14, 2023.
Application No. 18/334,909 is a continuation of application No. PCT/IL2021/051503, filed on Dec. 19, 2021.
(60) Provisional application No. 63/127,212, filed on Dec. 18, 2020.

(51) Int. Cl. C12N 15/113; A61P 25/00; G16B 30/10

(52) U.S. Cl. CPC C12N 15/1137; A61P 25/00; G16B 30/10; C12N 2310/11; C12N 2310/321

(57) ABSTRACT

A method of increasing an amount of Chromodomain Helicase DNA Binding Protein 2 (CHD2) in a neuronal cell is provided. The method comprising introducing into the cell a nucleic acid agent directed at the last exon of human Chaserr, thereby increasing the amount of CHD2 in the neuronal cell.

### RELATED APPLICATIONS

This application claims the benefit of priority from U.S. Provisional Patent Application No. 63/127,212 filed Dec. 18, 2020 which is hereby incorporated in its entirety.

### SEQUENCE LISTING STATEMENT

The ASCII file, entitled 89180SequenceListing.txt, created on Dec. 19, 2021, comprising 61,440 bytes, submitted concurrently with the filing of this application is incorporated herein by reference.

### FIELD AND BACKGROUND OF THE INVENTION

The present invention, in some embodiments thereof, relates to compositions for use in the treatment of CHD2 haploinsufficiency and methods of identifying same.

Chromodomain Helicase DNA Binding Protein 2 (Chd2) gene encodes an ATP-dependent chromatin-remodeling enzyme, which together with CHD1 belongs to subfamily I of the chromodomain helicase DNA-binding (CHD) protein family. Members of this subfamily are characterized by two chromodomains located in the N-terminal region and a centrally located SNF2-like ATPase domain [Tajul-Arifin, K. et al. Identification and analysis of chromodomain-containing proteins encoded in the mouse transcriptome. Genome Res. 13, 1416-1429 (2003)], and facilitate disassembly, eviction, sliding, and spacing of nucleosomes [Narlikar, G. J., Sundaramoorthy, R. & Owen-Hughes, T. Mechanisms and functions of ATP-dependent chromatin-remodeling enzymes. Cell 154, 490-503 (2013)].

In humans, CHD2 haploinsufficiency is associated with neurodevelopmental delay, intellectual disability, epilepsy, and behavioral problems [reviewed in Lamar, K.-M. J. & Carvill, G. L. Chromatin remodeling proteins in epilepsy: lessons from CHD2-associated epilepsy. Front. Mol. Neurosci. 11, 208 (2018)]. Studies in mouse models and cell lines also implicate Chd2 in neuronal dysfunction.

In all described cases, these individuals are haploinsufficient for CHD2, and so bear an intact WT copy of CHD2. Therefore, increase of CHD2 expression through perturbation of Chaserr, e.g., by using antisense oligonucleotides, might have a therapeutic benefit.

Multiple lines of evidence point to a strong link between long non-coding RNA (lncRNA) functions and those of chromatin-modifying complexes [Han, P. & Chang, C.-P. Long non-coding RNA and chromatin remodeling. RNA Biol. 12, 1094-1098 (2015)]. Numerous chromatin modifiers have been reported to interact with lncRNAs [Han et al., supra]. In addition, lncRNAs in vertebrate genomes are enriched in the vicinity of genes that encode for transcription-related factors [Ulitsky, I., Shkumatava, A., Jan, C. H., Sive, H. & Bartel, D. P. Conserved function of lincRNAs in vertebrate embryonic development despite rapid sequence evolution. Cell 147, 1537-1550 (2011)], including numerous chromatin-associated proteins, but the functions of the vast majority of these lncRNAs remain unknown.

Previous work by the present inventors discloses the presence of Chaserr a conserved lncRNA located upstream of Chd2 (Rom et al. Nature Communications 2019 10:5092): 1810026B05Rik in mouse (denoted as Chaserr, for CHD2 adjacent, suppressive regulatory RNA) and LINC01578/LOC100507217 in human (CHASERR), are almost completely uncharacterized lncRNAs, found upstream of and transcribed from the same strand as Chd2.

Chaserr acts in concert with the CHD2 protein to maintain proper Chd2 expression levels. Loss of Chaserr in mice leads to early postnatal lethality in homozygous mice, and severe growth retardation in heterozygotes. Mechanistically, loss of Chaserr leads to substantially increased Chd2 mRNA and protein levels, which in turn lead to transcriptional interference by inhibiting promoters found downstream of highly expressed genes. Chaserr production represses Chd2 expression solely in cis, and that the phenotypic consequences of Chaserr loss are rescued when Chd2 is perturbed as well. Targeting Chaserr is thus a potential strategy for increasing CHD2 levels in haploinsufficient individuals.

Additional background art includes:

www(dot)iscb(dot)org/cms_addon/conferences/ismb2020/postersdotphp?track=RegSys%20COS
I&session=B
github(dot)com/lncLOOM/lncLOOM

### SUMMARY OF THE INVENTION

According to an aspect of some embodiments of the present invention there is provided a method of increasing an amount of Chromodomain Helicase DNA Binding Protein 2 (CHD2) in a neuronal cell, the method comprising introducing into the cell a nucleic acid agent that down-regulates activity or expression of human Chaserr, wherein the nucleic acid agent is directed at the last exon of human Chaserr, thereby increasing the amount of CHD2 in the neuronal cell.

According to an aspect of some embodiments of the present invention there is provided a method of treating a disease or medical condition associated with Chromodomain Helicase DNA Binding Protein 2 (CHD2) haploinsufficiency in a subject in need thereof, the method comprising administering to the subject a therapeutically effective amount of a nucleic acid agent that down-regulates activity or expression of human Chaserr, wherein the nucleic acid agent is directed at the last exon of human Chaserr, thereby treating the disease or medical condition associated with CHD2 haploinsufficiency.

According to an aspect of some embodiments of the present invention there is provided a nucleic acid agent that down-regulates activity or expression of human Chaserr for use in treating a disease or medical condition associated with Chromodomain Helicase DNA Binding Protein 2 (CHD2) haploinsufficiency in a subject in need thereof, wherein the nucleic acid agent is directed at the last exon of human Chaserr.

According to some embodiments of the invention, the human Chaserr comprises an alternatively spliced variant selected from the group consisting of SEQ ID NO: 11 (NR_037600), SEQ ID NO: 12 (NR_037601), and SEQ ID NO: 13 (NR_037602).

According to some embodiments of the invention, the nucleic acid agent hybridizes to a nucleic acid sequence element which comprises SEQ ID NO: 2 (AUGG).

According to some embodiments of the invention, the nucleic acid agent hybridizes to a nucleic acid sequence element selected from the group consisting of AAGAUG (SEQ ID NO: 5) and AAAUGGA (SEQ ID NO: 6).

According to some embodiments of the invention, the nucleic acid agent hybridizes to a nucleic acid sequence element comprising AAGAUG (SEQ ID NO: 5) and/or AAAUGGA (SEQ ID NO: 6).

According to some embodiments of the invention, the nucleic acid agent inhibits binding of DHX36 to Chaserr.

According to some embodiments of the invention, the nucleic acid agent is an antisense oligonucleotide.

According to some embodiments of the invention, the antisense oligonucleotide has a nucleobase sequence as set forth in SEQ ID NO: 92-99 (where T is replaced with U).

According to some embodiments of the invention, the nucleic acid agent is an RNA silencing agent.

According to some embodiments of the invention, the nucleic acid agent is a genome editing agent.

According to some embodiments of the invention, the nucleic acid agent is active in an inducible manner.

According to some embodiments of the invention, the nucleic acid agent is active in a tissue or cell-specific manner.

According to some embodiments of the invention, the disease or medical condition associated with Chromodomain Helicase DNA Binding Protein 2 (CHD2) haploinsufficiency is selected from the group consisting of intellectual disability, autism, epilepsy and Lennox-Gastaut syndrome (LGS).

According to an aspect of some embodiments of the present invention there is provided a method of analyzing a set of sequences describing a plurality of homologous polynucleotides, the method comprising:
constructing a graph having a plurality of nodes arranged in layers, and a plurality of edges connecting nodes of consecutive layers, wherein each layer represents a sequence of the set such that a first layer represents a sequence describing a query polynucleotide, each node represents a k-mer within a respective sequence, and each edge connects nodes representing identical or homologous k-mers, k being from 6 to 12;
searching the graph for continuous non-intersecting paths along edges of the graph; and
generating an output identifying a k-mer corresponding to at least one path as a nucleic acid sequence of functional interest.

According to some embodiments of the invention, the method comprises, before the generating the output, iteratively repeating the constructing and the searching, each time for a shorter k-mer.

According to some embodiments of the invention, the method comprises, at each iteration cycle, applying paths obtained in a previous iteration cycle as constraints for the search.

According to some embodiments of the invention, the searching comprises applying a path depth criterion as a constraint for the search, such that the search is preferential for deeper paths than for shallower paths.

According to some embodiments of the invention, the searching comprises applying an Integer Linear Program (ILP) to the graph.

According to some embodiments of the invention, the homologous polynucleotides are DNA sequences.

According to some embodiments of the invention, the homologous polynucleotides are RNA sequences.

According to some embodiments of the invention, the method comprises aligning the sequences in the set according to a predetermined order, so as to provide a multiple alignment with multiple alignment layers, where a first layer is the query polynucleotide of the plurality of homologous polynucleotides, and wherein the multiple alignment layers respectively correspond to the layers of the graph.

According to some embodiments of the invention, the predetermined order is evolution-dictated, optionally wherein the query is the most advanced in evolution is the homologous polynucleotides.

According to some embodiments of the invention, a homology among the homologous k-mers is at least 70%.

According to some embodiments of the invention, the homologous polynucleotides comprise partial sequences.

According to some embodiments of the invention, the homologous polynucleotides are selected from the group consisting of 3′UTR, lncRNA and enhancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B provides an overview of an embodiment for discovering nucleic acid sequence elements referred to as the “LncLOOM” framework. (A) Overview of the LncLOOM methodology. LncLOOM processes ordered lists of sequences and recovers a set of ordered motifs conserved to various depths that can be further annotated as miRNA or RBP binding sites. (B) Schematic diagram of graph construction and motif discovery using integer linear programming (ILP) to find long non-intersecting paths. Sequences are ordered with monotonically increasing evolutionary distance from the top layer (human). BLAST high-scoring pairs (HSPs) that can be used to constrain the placement of edges (see Methods), are depicted as pink and red blocks beneath each sequence. The graph is used for construction of an ILP problem and its solution is used for construction of a set of long paths that correspond to conserved syntenic motifs (SEQ ID NOs: 29-32).

FIGS. 2A-F depict the discovery of conserved elements in the Cyrano lncRNA. (A) Outline of the genomic organization of Cyrano exons in select species. (B) Sequence elements identified by LncLOOM to be conserved in Cyrano in at least 17 species. The region containing elements found in the region alignable by BLAST between human and zebrafish Cyrano sequences is circled. Numbers between elements indicate the range distances between the elements in the 18 species. The circled number above each element indicates the element number used in the text and in the other panels. (C) Pairing between the predicted binding elements in Cyrano and the miR-25/92 and miR-7 miRNAs. (D) Evidence for binding of PUM1 and PUM2 to the UGUAUAG motif (shaded region) in the human genome. ENCODE project CLIP data (top, K562 cells) and ²² (bottom, HCT116 cells). Shading is based on strength of binding evidence, as defined by the ENCODE project. (E) Binding and regulation of the mouse Cyrano sequence by Pum1/2 and Rbfox1/2. Top: Pum1/2 CLIP and RNA-seq data from. Middle: Rbfox1 CLIP from mouse brain and from mESCs. Binding motifs for Pumilio and Rbfox are highlighted in yellow and blue, respectively. PhyloP sequence conservation scores are from the UCSC genome browser. Bottom: Binding of Ago2 in the mouse brain to the region of the miR-153 binding site near the 3′ end of Cyrano. CLIP data from (F) Top left: Alignment of the region surrounding the conserved AUGGCG motif near the 5′ end of Cyrano. Top right and bottom: Composite Ribo-seq and RNA-seq data from multiple datasets curated in. Chip-seq data for YY1 in the K562 cell line from the ENCODE project. Shown is the read coverage and the IDR peaks. Sequences shown in the panels are marked as SEQ ID NOs: 33-42 and 53-67.

FIG. 3A-E depict the discovery of conserved elements in the CHASERR lncRNA. (A) Human CHASERR gene structure is shown with motifs conserved in at least four species color-coded by their depth of conservation. The region of the last exon is magnified, and the motifs discussed in the text are highlighted. (B) Sequence logos of the sequences flanking the two most conserved motifs, with the shared AARAUGR motif shaded (a sequence shown in the panel is marked as SEQ ID NO: 68). (C) Top: mouse Chaserr locus with the positions of the primer pairs used for qRT-PCR, and the regions targeted by the GapmeRs (the same ones as used in) and ASOs highlighted. Bottom: qRT-PCR with primers targeting Chaserr (shown on top) or Chd2 exons in N2a cells treated with the indicated reagents, n=4 for ASO treatments and n=5 for GapmeRs. (D) Volcano plot for comparison of MS intensities between pulldown with the WT sequence of the Chaserr last exon and the last exon where the conserved elements were mutated (FIG. 8A). (E) qRT-PCR using primers targeting the indicated regions following IP with the indicated antibody, n=4. Top right: Western blot using anti-DHX36 antibody on the indicated sample. A sequence shown in the Figure is marked as SEQ ID NO. 68.

FIG. 4 shows the identification of conserved elements in the PUM1 and PUM2 3′UTRs. The human sequence is shown and the motifs conserved in at least seven species are color-coded based on their conservation. The occurrences of the ultra-conserved UGUACAUU (SEQ ID NO: 14) motif are in a box. Sequences shown in the panel are marked as SEQ ID NOs: 69-70.

FIGS. 5A-I show Global analysis of conserved motifs in 3′UTRs with LncLOOM. (A) Number of genes with various numbers of ortholog sequences that had no significant alignment to their human sequence (black) or to their mouse, dog and chicken sequences (grey). (B) Distribution of combinations of unique k-mers conserved in the indicated number of sequences that did not align to the human 3′UTR sequence. (C) Quantification of the total number of unique k-mers (pink) and their total instances (dark red) that LncLOOM identified per species. The total number of broadly conserved miRNA binding sites is shown in green, and the number of unique k-mers that correspond to these sites in yellow. The number of genes that contained any k-mer is shown in grey, and the number of genes that contained at least one k-mer that correspond to a miRNA site is shown in black. (D) Top: Distribution of unique k-mers that were identified in the first sequence non-alignable to human in multiple genes (grey). The number of k-mers detected in an invertebrate species in at least one gene is shown in black. Bottom: Unique k-mers common to at least 50 genes and detected in an invertebrate sequence. k-mers that resemble an ARE are coloured red, those resembling a PAS are blue and those resembling a PRE are green. (E) Comparison of genes that contained broadly conserved miRNA binding sites detected by LncLOOM and TargetScan in the human sequences of genes analysed. (F) Number of broadly conserved miRNA bindings detected by LncLOOM per number of non-alignable sequences; the percentage of genes with a miRNA site detected per number of non-alignable layers (black) and the number of unique k-mers corresponding to the miRNA binding sites (yellow). (G) Top: Broadly conserved miRNA binding sites predicted by LncLOOM in human sequences. Sites predicted by TargetScan and recovered by LncLOOM are shown in red, and new sites in blue. Bottom: The conservation of these sites per number of species. (H) Comparison of the fractions of genes with at least one miRNA site detected in the indicated species by TargetScan and LncLOOM. Only sites found in TargetScanHuman were used. (I) Percentage of genes that contain a miRNA site detected by LncLOOM per number of non-alignable sequences: (red) miRNA sites that were previously predicted by TargetScan in the human sequence and recovered by LncLOOM in additional sequences, that were not part of the MSA used by TargetScan; (blue) new miRNA sites predicted in by LncLOOM but not previously predicted by TargetScan in the human sequences.

FIG. 6 show conserved elements in the libra lncRNA. The human sequence is shown and the motifs conserved in at least five species are color-coded based on their conservation. Pairs of vertical lines represent intron positions. Motifs that match miRNA seed sites are indicated with the miRNA family name above the motif. Regions that are part of BLASTN alignments (E<0.001) between the human and spotted gar sequences are underlined. A sequence shown in the panels is marked as SEQ ID NO: 71.

FIG. 7 show gaps in the genomic assembly around the first exon in the Chaserr lncRNA locus. For each species, RNA-seq read coverage is shown, alongside gaps in the genome assembly (from the UCSC browser).

FIGS. 8A-D show functional characterization of the conserved elements in Chaserr lncRNA. (A) Sequence of the last exon of mouse Chaserr. The deeply conserved elements are shared. The conserved AUGG instances that were mutated in the MS baits are in blue and all the other AUGG instances are in green. Regions targeted by the ASOs are marked. (B) As in FIG. 3C, for the indicated ASO treatments. (C) RNA-seq quantification of the expression of the indicated gene in HEK293 cells with the indicated genotype, data from (D) RNA-seq quantification of the expression of the indicated genes in THPI cells treated with a non-targeting shRNA (shNT) or a shRNA targeting ZFR. Data from The sequence shown in 8A is marked as SEQ ID NO: 72.

FIG. 9 shows the identification of conserved elements in the DICER 3′UTRs. The human sequence is shown and the motifs conserved in at least eight vertebrate species are color-coded based on their conservation (9 species-conserved in lancelet; 10 species-conserved in lancelet and sea urchin). Regions of motifs for which 100 random sequences preserving sequence identity do not contain any motif of this length are shaded in light yellow. Regions of motifs for which in random sequences the exact motif is not found are shaded in light cyan. A sequence shown in the panel is marked as SEQ ID NO: 73.

FIGS. 10A-F show additional analysis of LncLOOM motifs identified in 3′UTRs. (A) Distribution of orthologous 3′UTR sequences. Top left: Frequency of genes that were analysed at various depths. Top right: Distribution of various combinations of non-amniote sequences that were included in the 3′UTR sequence datasets. Bottom right: Overall number of genes analyzed in the indicated species. (B) Distribution of combinations of unique k-mers conserved per number of non-alignable sequences in 3′UTR datasets. Alignments to human, mouse, dog and chicken were considered. (C) Distribution of unique k-mers that were identified beyond amniotes and shared between multiple genes. Number of k-mers containing UUU (red line), AUAA (green line) or that matched a broadly conserved miRNA site (yellow line) are indicated. (D) Conservation of broadly conserved miRNA sites that were detected by LncLOOM in genes for which TargetScan did not report any predictions. (Top) Number of genes with a miRNA site detected per number of species (left) and number of non-alignable sequences (right). (Bottom left) Number of genes with a miRNA site detected per species. (Middle) Number of new miRNA sites detected per species. (Right) Number of new miRNA sites detected per number of non-alignable sequences. (E) Comparison of miRNA sites that have conservation detected per species by TargetScan and LncLOOM. Only sites that were previously identified by TargetScanHuman have been compared. (F) Conservation of miRNA sites detected by LncLOOM in sequences that had no alignment to the human sequence. Sites that were previously predicted by TargetScan in the human sequence are coloured red and new LncLOOM predictions are coloured blue.

FIGS. 11A-D show the constraints imposed on the LncLOOM graph. (A) Examples of scenarios in the LncLOOM graph and how those are represented in the ILP. (B) Conditional constraint on intersecting edges. An example of the suboptimal exclusion of repeated k-mers in complex paths during refinement in subsequent iterations that can occur if all intersections are constrained. (C) Flow diagram for defining conditional constraints on intersecting edges: a pair of intersecting edges is only constrained if there is at least one other edge, from a unique path, that intersects either of the edges. (D) Example demonstrating how the conditional constraint on intersections can mitigate the suboptimal exclusion of tandemly repeated k-mers. A sequence shown in the panel is marked as SEQ ID NO: 74.

FIG. 12 shows the Partitioning of the LncLOOM graph and iterative refinement of selected repeated k-mers. Starting with the deepest layer in the graph, motif discovery is performed through an iterative process in which each step searches for motifs that are conserved at an increasingly shallower depth. Shown here is an example of motif discovery that begins in a graph of 5 layers. The graph is solved and the simple paths obtained in the solution (shown in green) are then used to partition the graph into subgraphs that are solved individually in the next iteration, which is performed on the top 4 layers of the graph. Each simple path is immediately added to the final solution, while complex paths (shown in blue and red) are refined during the subsequent iterations of motif discovery. In this case, the repeated k-mers that are removed during optimization are circled in pink.

FIGS. 13A-B show processing steps in the LncLOOM framework. (A) Construction of the 5′ and 3′ graphs. LncLOOM uses the median positions of the first and last motifs identified in the primary ILP (in which the full-length of each sequence is considered) to predict and extract the 5′ and 3′ ends of individual sequences that are extended relative to other sequences in the graph. LncLOOM motif discovery is then performed on the subset of extracted 5′ and 3′ regions. In this example a minimum depth of 3 has been imposed, thus the AUUGCU (SEQ ID NO: 15, blue) motif that is only conserved in the top 2 sequences is ignored, and the CAUCCA (SEQ ID NO: 16, dark red and underlined) is considered as the first node instead. (B) Illustration of motif neighbourhoods. The reference sequence of each neighbourhood is determined by combining all overlapping k-mers in the anchor sequence. All k-mers that are conserved to respective depths in the graph and which are connected to one of the overlapping k-mers within the reference sequence, are then included within the neighbourhood. Sequences shown in the panels are marked as SEQ ID NO: 75-87.

FIG. 14 is a flowchart diagram of a method suitable for analyzing a set of sequences, according to various exemplary embodiments of the present invention.

FIG. 15 is a schematic illustration of a computing platform configured for analyzing a set of sequences, according to various exemplary embodiments of the present invention.

FIG. 16 is a graphic display of changes in gene expression, relative to untransfected SH-SY5Y cells, of CHASERR, CHD2, and p21 (CDKNIA) following transfection of the indicated ASOs (SEQ ID Nos: 128 and 134).

FIG. 17 is a graphic display of changes in gene expression, relative to untransfected MCF7 cells and SH-SY5Y cells, of CHASERR and CHD2 following transfection of the indicated ASOs (SEQ ID Nos: 128 and 134).

### DESCRIPTION OF SPECIFIC EMBODIMENTS OF THE INVENTION

The present invention, in some embodiments thereof, relates to compositions for use in the treatment of CHD2 haploinsufficiency and methods of identifying same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

CHD2 haploinsufficiency is associated with neurodevelopmental delay, intellectual disability, epilepsy, and behavioral problems. Previous results show that CHD2 expression is tightly regulated by Chaserr, a conserved lncRNA located upstream of Chd2. Loss of Chaserr leads to substantially increased Chd2 mRNA and protein levels, which in turn lead to changes in gene expression, including transcriptional interference by inhibiting promoters found downstream of highly expressed genes.

Whilst conceiving embodiments of the invention, the present inventor have devised a novel algorithm for the detection of conserved elements in sequences that have diverged beyond alignability and/or have accumulated substantial lineage-specific sequences such as transposable elements. Using this algorithm, or an embodiment thereof referred to as “LncLOOM”, the present inventors have identified, and validated conserved regions of Chaserr that can be preferentially mutated/targeted to specifically inhibit interactions of Cheserr with functionally-relevant interactors and compensate eventually for CHD2 haploinsufficiency.

Thus, according to an aspect of the invention, there is provided a method of increasing an amount of Chromodomain Helicase DNA Binding Protein 2 (CHD2) in a neuronal cell, the method comprising introducing into the cell a nucleic acid agent that down-regulates activity or expression of human Chaserr, wherein the nucleic acid agent is directed at the last exon of human Chaserr, thereby increasing the amount of CHD2 in the neuronal cell.

As used herein “a nucleic acid agent that down-regulated activity or expression of human Chaserr” refers to an nucleic acid molecule that inhibits activity or reduces the amount of human Chaserr.

According to some embodiments, “a nucleic acid agent that down-regulates activity of human Chaserr”, includes any one or more of, a nucleic acid agent that increases the expression (protein and optionally mRNA) of CHD2, a nucleic acid agent that increases the stability of CHD2 mRNA, a nucleic acid agent that induces expression of CHD2 mRNA, and a nucleic acid agent that induces translation of CHD2.

Thus, according to an aspect of the invention there is provided a nucleic acid agent that down-regulates activity or of human Chaserr, wherein the nucleic acid agent comprises a nucleic acid sequence that hybridizes at (i.e., is complementary to a nucleotide sequence within) the last exon of human Chaserr.

As used herein “Chromodomain Helicase DNA Binding Protein 2 (CHD2)” refers to an enzyme that in humans is encoded by the CHD2 gene. Examples of CHD2 splice variants in humans include NCBI Reference Sequence: NM_001271.4 and NM_001042572.

The splice variant protein product is as set forth in NCBI Reference Sequence: NP_001262.3 or NP_001036037.

As used herein “haploinsufficiency refers to a model of dominant gene action in diploid organisms, in which a single copy of the standard (so-called wild-type) allele at a locus in heterozygous combination with a variant allele is insufficient to produce the standard phenotype. Typically, only about half of the amount of the protein is produced as compared to the healthy condition where both alleles are of the wild-type form.

As used herein “increasing the amount” refers to increasing the amount of a protein or RNA of interest by a statistically significant amount, and an amount that has utility for treating haploinsufficiency of the protein or RNA of interest. In various embodiments, “increasing the amount” of a protein or RNA of interest involves an increase of at least 10%, or in some embodiments, at least about 20%, at least 20%, 20-150%, 50-150%, e.g., by at least, 50%, 60%, 70%, 80%, 90%, 1.2 fold 1.4 fold 1.5 fold or more e.g., at least 2 fold. According to a specific embodiment, the CHD2 levels are restored to the amount found in a normal cell (without the haploinsufficiency) of the same type (i.e., neuronal) and developmental stage.

As used herein “neuronal cell” refers to a cell that is found in the subject's body (in-vivo), or outside the body, such as a tissue biopsy, cell-line and primary culture.

Other cells are also contemplated, i.e., non-neuronal cells.

The neuronal cell may be genetically modified or non-genetically modified, e.g., naive.

According to a specific embodiment, the neuronal cell is located in the central nervous system.

Methods of qualifying cells in which the level of CHD2 is to be or was modified according to some embodiments of the invention, are well known in the art.

Contacting cells with the agent can be performed by any in-vivo or in-vitro conditions including for example, adding the agent to cells derived from a subject (e.g., a primary cell culture, a cell line) or to a biological sample comprising same (e.g., a fluid, liquid which comprises the cells) such that the agent is in direct contact with the cells. According to some embodiments of the invention, the cells of the subject are incubated with the agent. The conditions used for incubating the cells are selected for a time period/concentration of cells/concentration of agent/ratio between cells and agent and the like which enable the drug to induce cellular changes such as increase in the level (amount) of CHD2 or associated changes such as changes in transcription and/or translation rate of specific genes, proliferation rate, differentiation, cell death, necrosis, apoptosis and the like.

The level of CHD2 (mRNA and/or protein) can be analyzed prior to, concomitant with and/or following introducing the agent into the cell. Additionally or alternatively, the genomic DNA is analyzed for the modification introduced by the agent, as further described hereinbelow such as in the case of genome editing.

Down-regulation at the nucleic acid level (i.e., reduced abundance of a nucleic acid) is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

It will be appreciated that the nucleic acid agents are contemplated herein per se, encoded from a nucleic acid construct or as part of a pharmaceutical composition.

According to specific embodiments, the downregulating agent is a polynucleotide or oligonucleotide capable of hybridizing to a gene or mRNA encoding CHD2.

According to specific embodiments, the downregulating agent directly interacts with the gene of CHD2 or the RNA transcription product.

According to specific embodiments, the agent directly binds a nucleic acid sequence within the last exon of Chaserr.

As used herein “Chaserr” refers to CHD2 Adjacent Suppressive Regulatory RNA. HGNC: 48626 Entrez Gene: 100507217

Exon organization of Chaserr is as follows: EXON1: nucleotides 1 . . . 344; EXON2: nucleotides 345 . . . 538; EXON3: nucleotides 539 . . . 608; EXON4: nucleotides 609 . . . 694; EXON5: nucleotides 695 . . . 763; EXON6: nucleotides 764 . . . 1787, wherein the last exon of Chaserr refers to nucleotides 764 . . . 1787 of SEQ ID NO: 3 (NR_037601).

According to a specific embodiment, the nucleic acid agent hybridizes to a nucleic acid sequence element which comprises SEQ ID NO: 1 (AUG).

According to another embodiment, the nucleic acid agent hybridizes to a nucleic acid sequence element which comprises SEQ ID NO: 2 (AUGG).

According to a specific embodiment, the nucleic acid agent hybridizes to a nucleic acid sequence element comprising AAGAUGG (SEQ ID NO: 4), AAGAUG (SEQ ID NO: 5) or AAAUGGA (SEQ ID NO: 6).

According to another embodiment, the nucleic acid agent hybridizes to a nucleic acid sequence element which comprises SEQ ID NO: 3 (aauaaa).

According to a specific embodiment, the nucleic acid agent inhibits binding of DHX36 to Chaserr.

As used herein “DHX36” refers to probable ATP-dependent RNA helicase DHX36 also known as DEAH box protein 36 (DHX36) or MLE-like protein 1 (MLEL1) or G4 resolvase 1 (G4R1) or RNA helicase associated with AU-rich elements (RHAU) is an enzyme that in humans is encoded by the DHX36 gene.

According to a specific embodiment, the nucleic acid agent comprises a nucleotide sequence that is complementary to UUUUUACCU (SEQ ID NO: 122)

According to a specific embodiment, the nucleic acid agent inhibits binding of CHD2 to Chaserr.

According to specific embodiments the downregulating agent is an antisense, RNA silencing agent or a genome editing agent.

According to a specific embodiment, the downregulating agent is an antisense.

Antisense oligonucleotide—Antisense oligonucleotide is a single stranded oligonucleotide designed to hybridize to a target RNA, thereby inhibiting its function or levels. Downregulation or inhibition of a Chaserr RNA can be effected using an antisense oligonucleotide capable of specifically hybridizing with an Chaserr transcript e.g., comprising SEQ ID NO: 1, 2, 4, or 6. Preferably, hybridization of the antisense oligonucleotide prevents binding of an effector element to Chaserr but otherwise leaves the Chaserr RNA intact. According to a specific embodiment, the nucleic acid agent does not recruit RNaseH. In some embodiments, the antisense oligonucleotide does not recruit RNaseH. For example, the antisense oligonucleotide may comprise substantially RNA nucleotides. In still other embodiments, the antisense oligonucleotide recruits RNaseH, and thus comprises at least a stretch of DNA nucleotides. For example, the antisense oligonucleotide may be a gapmer.

According to a specific embodiment, the antisense sequences corresponding to the antisense oligonucleotides (ASOs) that are exampled for mouse in the Examples section which follows include, but are not limited to, CCATAGTAGACTGCCATCTT (SEQ ID NO: 7) targeting AAGATGGCAGTCTACTATGG (SEQ ID NO: 12) and ATCCACTGTCCATTTGTG (SEQ ID NO: 9) targeting CACAAATGGACAGTGGAT (SEQ ID NO: 10). While nucleotide sequences are presented here as full DNA or RNA sequences for convenience, it is understood that antisense oligonucleotides can be constructed as either RNA or DNA nucleotides, or mixtures thereof. That is, where an oligonucleotide indicates the nucleotide thymine (T), it is understood that the nucleotide can be replaced with its RNA counterpart (uridine, or U), and vice versa. Further, it is understood that DNA and RNA nucleotide modifications, such as those well known in the art, can be used to construct the antisense oligonucleotides.

According to a specific embodiment, the nucleic acid agent comprises a nucleotide sequence that is complementary to UUUUUACCU (SEQ ID NO: 122). As used herein, the term “complementary” refers to canonical (A/T, A/U, and G/C) base-pairing.

According to a specific embodiment, the nucleic acid agent inhibits binding of CHD2 to Chaserr.

According to a specific embodiment, the antisense oligonucleotide has a nucleobase sequence as set forth in SEQ ID NO: 140-143, (corresponding to A40, 50, 51, 52). In the modified version thereof it is provided as SEQ ID Nos: 128, 131, 132 and 133.

Design of antisense molecules which can be used to efficiently inhibit or reduce the amount of Chaserr must be effected while considering two aspects important to the antisense approach. The first aspect is delivery of the oligonucleotide into the nucleus of the appropriate cells, while the second aspect is design of an oligonucleotide which specifically binds the designated RNA within cells in a way which inhibits the desired function.

The prior art teaches of a number of delivery strategies which can be used to efficiently deliver oligonucleotides into a wide variety of cell types [see, for example, Jaaskelainen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65]

In addition, algorithms for identifying those sequences with the highest predicted binding affinity for their target RNA based on a thermodynamic cycle that accounts for the energetics of structural alterations in both the target RNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

For example, suitable antisense oligonucleotides targeted against the Chaserr RNA would be of the sequences listed in Table 3 below (and is considered an integral part of the specification) or any of the antisense oligonucleotides as set forth in SEQ ID NO: 140-143 or with modifications set forth in SEQ ID Nos: 128, 131, 132 or 133, corresponding to A40, 50, 51, 52.

In accordance with various embodiments, the antisense oligonucleotide can comprise fully RNA nucleotides. Such antisense oligonucleotides will not recruit RNaseH, and thus, Chaserr should not be degraded by the antisense inhibition thereof. In still other embodiments, the antisense oligonucleotide comprises a mix of DNA and RNA nucleotides (e.g., a gapmer), which is able to recruit RNaseH and degrade Chaserr RNA.

In some embodiments, the antisense oligonucleotide comprises one or more nucleotides containing a 2′ to 4′ bridge, such as a locked nucleotide (LNA) or a constrained ethyl (cEt), and other bridged nucleotides described herein.

In some embodiments, the antisense oligonucleotide comprises one or more (or all in some embodiments) of nucleotides having a 2′-0 modification, such as 2′-OMe or 2′-O-methoxyethyl (2′-O-MOE).

In some embodiments, the antisense oligonucleotide comprises a modified backbone, such as phosphorothioate, or phosphorodithioate. In still other embodiments, the antisense oligonucleotide comprises a morpholino backbone.

In some embodiments, the antisense oligonucleotide comprises one or more nucleotides having modified bases, such as 5-methyl cytosine.

Other nucleotide modifications that can be employed are described elsewhere herein.

Alternatively, downregulation of CHD2 can be achieved by RNA silencing.As used herein, the phrase “RNA silencing” refers to a group of regulatory mechanisms [e.g. RNA interference (RNAi), transcriptional gene silencing (TGS), post-transcriptional gene silencing (PTGS), quelling, and co-suppression]mediated by RNA molecules which result in the inhibition or “silencing” of the RNA activity or availability. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

According to an embodiment of the invention, the RNA silencing agent is specific to the target RNA and in fact to a nucleic acid region which includes the last exon of Chaserr (as described hereinabove with the following elements: e.g., SEQ ID NO: 1, 2, 4 or 6) and does not cross inhibit or silence other targets (or other exons in the same target) which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al.,Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433 and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include are listed in International Patent Application Nos. WO2013126963 and WO2014107763. It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the Chaserr mRNA sequence is scanned for AA dinucleotide sequences. Occurrence of each AA and the 3′ adjacent 19 nucleotides is recorded as potential siRNA target sites.

Second, potential target sites are compared to an appropriate genomic database (e.g., human, mouse, rat etc.) using any sequence alignment software, such as the BLAST software available from the NCBI server (www(dot)ncbi.nlm.nih(dot)gov/BLAST/).

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses(dot)fwdarw(dot)humans) and have been shown to play a role in development, homeostasis, and disease etiology.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

Nucleic acid sequence modifications are also contemplated herein to improve bioavailability, affinity, stability or combination thereof.

According to one embodiment, the nucleic acid agent includes at least one base (e.g. nucleobase) modification or substitution.

As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990), “The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and O-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications. Additional base modifications are described in Deleavey and Damha, Chemistry and Biology (2012) 19: 937-954, incorporated herein by reference.

According to one embodiment, the modification is in the backbone (i.e. in the internucleotide linkage and/or the sugar moiety).

Sugar modification of nucleic acid molecules have been extensively described in the art (see PCT International Publication Nos. WO 92/07065, WO 93/15187, WO 98/13526, and WO 97/26270; U.S. Pat. Nos. 5,334,711; 5,716,824; and U.S. Pat. No. 5,627,053; Perrault et al., 1990; Pieken et al., 1991; Usman & Cedergren, 1992; Beigelman et al., 1995; Karpeisky et al., 1998; Earnshaw & Gait, 1998; Verma & Eckstein, 1998; Burlina et al., 1997; all of which are incorporated herein by reference). Such publications describe general methods and strategies to determine the location of incorporation of sugar, base, and/or phosphate modifications and the like into nucleic acid molecules without modulating catalysis. Exemplary sugar modifications include, but are not limited to, 2′-modified nucleotide, e.g., a 2′-deoxy, 2′-fluoro (2′-F), 2′-deoxy-2′-fluoro, 2′-O-methyl (2′-O-Me), 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-Fluoroarabinooligonucleotides (2′-F-ANA), 2′-O—N-methylacetamido (2′-O-NMA), 2′-NH2 or a locked nucleic acid (LNA). Additional sugar modifications are described in Deleavey and Damha, Chemistry and Biology (2012) 19: 937-954, incorporated herein by reference.

Thus, for example, oligonucleotides can be modified to enhance their stability and/or enhance biological activity by modification with nuclease resistant groups, for example, the Nucleic acid agent of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), e.g. inclusion of nucleic acid analogues in which the ribose ring is “locked” by a methylene bridge connecting the 2′-0 atom and the 4′-C atom, ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to the target. The inclusion of pyranose sugars in the oligonucleotide backbone can also decrease endonucleolytic cleavage. The binding arms may further include peptide nucleic acid (PNA) in which the deoxribose (or ribose) phosphate backbone in the DNA is replaced with a polyamide backbone, or may include polymer backbones, cyclic backbones, or acyclic backbones. The binding regions may incorporate sugar mimetics, and may additionally include protective groups, particularly at terminal ends thereof, to prevent undesirable degradation (as discussed below).

Exemplary internucleotide linkage modifications include, but are not limited to, phosphorothioate, chiral phosphorothioate, phosphorodithioate, phosphotriester, aminoalkyl phosphotriester, methyl phosphonate, alkyl phosphonate (including 3′-alkylene phosphonates), chiral phosphonate, phosphinate, phosphoramidate (including 3′-amino phosphoramidate), aminoalkylphosphoramidate, thionophosphoramidate, thionoalkylphosphonate, thionoalkylphosphotriester, boranophosphate (such as that having normal 3′-5′ linkages, 2′-5′ linked analogues of these, and those having inverted polarity wherein the adjacent pairs of nucleoside units are linked 3′-5′ to 5′-3′ or 2′-5′ to 5′-2′), boron phosphonate, phosphodiester, phosphonoacetate (PACE), morpholino, amidate carbamate, carboxymethyl, acetamidate, polyamide, sulfonate, sulfonamide, sulfamate, formacetal, thioformacetal, alkylsilyl, substitutions, peptide nucleic acid (PNA) and/or threose nucleic acid (TNA). Various salts, mixed salts, and free acid forms of the above modifications can also be used. Additional internucleotide linkage modifications are described in Deleavey and Damha, Chemistry and Biology (2012) 19: 937-954; and Hunziker & Leumann, 1995 and De Mesmaeker et al., 1994, both incorporated herein by reference.

According to a specific embodiment, the modification comprises modified nucleoside triphosphates (dNTPs).

According to one embodiment, the modification comprises an edge-blocker oligonucleotide.

According to a specific embodiment, the edge-blocker oligonucleotide comprises a phosphate, an inverted dT and an amino-C7.

According to one embodiment, the nucleic acid agent is modified to comprise one or more protective group, e.g. 5′ and/or 3′-cap structures.

As used herein, the phrase “cap structure” is meant to refer to chemical modifications that have been incorporated at either terminus of the oligonucleotide (see e.g., U.S. Pat. No. 5,998,203, incorporated by reference herein). These terminal modifications protect the nucleic acid molecule from exonuclease degradation, and can help in delivery and/or localization within a cell. The cap modification can be present at the 5′-terminus (5′-cap) or at the 3′-terminal (3′-cap), or can be present on both termini. In non-limiting examples: the 5′-cap is selected from the group comprising inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In some embodiments, the 3′-cap is selected from a group comprising inverted deoxynucleotide, such as for example inverted deoxythymidine, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non-bridging methylphosphonate and 5′-mercapto moieties (see generally Beaucage & Iyer, 1993; incorporated by reference herein).

A nucleic acid agent can be further modified by including a 3′ cationic group, or by inverting the nucleoside at the terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3-5′-exonucleases.

According to one embodiment, the 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

According to a specific embodiment, the modification comprises inclusion of locked nucleic acids (LNA) or other bridged nucleotides such as cEt, and/or 2EC′2-Mehoxyethyl) abbreviated a 2′MOE) or 2′-OMe modifications, whereby at least part or all of the sequence is modified at the 2′ position of each nucleotide. Examples include, but are not limited to A40, A50, A51, A35, A49 and A52.

Also contemplated herein are gapmers (see Examples section which follows, see Table 5). A gapmer is a chimeric antisense oligonucleotide that contains a central block of deoxynucleotide monomers sufficiently long to induce RNase H cleavage.

Nucleic acid agents (as well as modifications thereof as described above) can also operate at the DNA level as summarized infra.

Downregulation of Chaserr can also be achieved by inactivating the gene (e.g., Chaserr) via introducing targeted mutations involving loss-of function alterations (e.g. point mutations, deletions and insertions) in the gene structure.

As used herein, the phrase “loss-of-function alterations” refers to any mutation in the DNA sequence of a gene (e.g., in the last exon of Chaserr) which results in downregulation of the expression level and/or activity of the expressed lncRNA product. Non-limiting examples of such loss-of-function alterations include, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes any nucleic acids in a gene sequence; an insertion mutation, i.e., a mutation which inserts nucleic acids into a gene sequence, and which may result in insertion of a transcriptional termination sequence; an inversion, i.e., a mutation which results in an inverted sequence; a splice mutation i.e., a mutation which results in abnormal splicing or poor splicing; and a duplication mutation, i.e., a mutation which results in a duplicated sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. mutation in the last exon of Chaserr may be in a homozygous form or in a heterozygous form.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264 and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Examples include genome editing agents such as CRISPR-Cas, Meganucleases, zinc finger nucleases (ZFNs), TALENs, use of transposons and the like.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

According to one embodiment, the present techniques relate to introducing the RNA silencing molecules using transient DNA or DNA-free methods (such as RNA transfection).

According to one embodiment, the RNA silencing molecule (e.g. antisense molecule) is delivered as a “naked” oligonucleotide, i.e. without the additional delivery vehicle. According to one embodiment, the “naked” oligonucleotide comprises a chemical modification to facilitate its tissue delivery (e.g. utilizing inverted nucleotides, phosphorothioate linkages, or integration of locked nucleic acids, as discussed above).

Any method known in the art for RNA or DNA transfection can be used in accordance with the present teachings, such as, but not limited to microinjection, electroporation, lipid-mediated transfection e.g. using liposomes, or using cationic molecules or nanomaterials (discussed below, and further discussed in Roberts et al. Nature Reviews Drug Discovery (2020) 19: 673-694, incorporated herein by reference).

According to one embodiment, and as mentioned above, in cases where the RNA silencing molecule (e.g. antisense) does not comprise a chemical modification it may be administered to the target cell (e.g. senescent cell) as part of an expression construct. In this case, the RNA silencing molecule (e.g. antisense molecule) is ligated in a nucleic acid construct (also referred to herein as an “expression vector”) under the control of a cis-acting regulatory element (e.g. promoter) capable of directing an expression of the RNA silencing molecule (e.g. antisense) in the target cells (e.g. neuronal cell) in a constitutive or inducible manner.

The expression constructs of the present invention may also include additional sequences which render it suitable for replication and integration in eukaryotes (e.g., shuttle vectors). Typical cloning vectors contain transcription and translation initiation sequences (e.g., promoters, enhances) and transcription and translation terminators (e.g., polyadenylation signals). The expression constructs of the present invention can further include an enhancer, which can be adjacent or distant to the promoter sequence and can function in up regulating the transcription therefrom. Polyadenylation sequences can also be added to the expression constructs of the present invention in order to increase the efficiency of expression.

In addition to the embodiments already described, the expression constructs of the present invention may typically contain other specialized elements intended to increase the level of expression of cloned nucleic acids or to facilitate the identification of cells that carry the RNA silencing molecule (e.g. antisense). The expression constructs of the present invention may or may not include a eukaryotic replicon.

The nucleic acid construct may be introduced into the target cells (e.g. neuronal cells) of the present invention using an appropriate gene delivery vehicle/method (transfection, transduction, etc.) and an appropriate expression system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Additionally or alternatively, lipid-based systems may be used for the delivery of constructs or nucleic acid agent encoded thereby into the target cells (e.g. senescent cells or cancer cells) of the present invention. Lipid bases systems include, for example, liposomes, lipoplexes and lipid nanoparticles (LNPs). In some embodiments, the antisense oligonucleotide or siRNA comprises a conjugated lipid or cholesteryl moiety,

Neuronal-specific promoters can be used to improve the specificity of the method. Examples of neuronal-specific promoters include, but are not limited to, synapsin. Synapsin is considered to be a neuron-specific protein (DeGennaro et al., 1983 Cold Spring Harb. Symp. Quant. Biol. 1, 337-345), so its neuron-specific expression pattern can be harnessed to express transgenes in a neuron-specific manner. A minimal human synapsin promoter has been used in adenoviral and AAV vectors for focal injections (Kugler et al. 2003 Human synapsin 1 gene promoter confers highly neuron-specific long-term transgene expression from an adenoviral vector in the adult rat brain depending on the transduced area. Gene Ther. 10, 337-347). An AAV capsid that can reach the CNS after peripheral administration, such as AAV9 or other natural AAV serotypes is advantageous for a relatively non-invasive administration that yields wide-scale expression. Now there are several engineered capsids with increased neuronal transduction efficiency. Lentivirus with E/SYN promoter has been reported to exhibit strong persistent expression in neurons (Hioki et al. Gene Therapy volume 14, pages 872-882(2007)).

The present teachings can be harnessed towards the clinic in the treatment of related diseases, syndromes, disorders and medical conditions associated with CHD2 haploinsufficiency.

Thus, according to an aspect of the invention there is provided a method of treating a disease or medical condition associated with Chromodomain Helicase DNA Binding Protein 2 (CHD2) haploinsufficiency in a subject in need thereof, the method comprising administering to the subject a therapeutically effective amount of a nucleic acid agent that down-regulates activity or expression of human Chaserr, wherein the nucleic acid agent is directed at the last exon of human Chaserr, thereby treating the disease or medical condition associated with CHD2 haploinsufficiency.

According to an alternative or an additional aspect there is provided a nucleic acid agent that down-regulates activity or expression of human Chaserr for use in treating a disease or medical condition associated with Chromodomain Helicase DNA Binding Protein 2 (CHD2) haploinsufficiency in a subject in need thereof, wherein the nucleic acid agent is directed at the last exon of human Chaserr.

As used herein “a disease or medical condition associated with Chromodomain Helicase DNA Binding Protein 2 (CHD2) haploinsufficiency” refers to a pathogenic condition which is characterized by-, or which onset or progression is associated with a reduced expression (protein and optionally mRNA) of CHD2.

According to a specific embodiment, the disease or medical condition associated with CHD2 haploinsufficiency refers to a CHD2-related neurodevelopmental disorder which is typically characterized by early-onset epileptic encephalopathy (i.e., refractory seizures and cognitive slowing or regression associated with frequent ongoing epileptiform activity). Seizure onset is typically between ages six months and four years. Seizure types typically include drop attacks, myoclonus, and a rapid onset of multiple seizure types associated with generalized spike-wave on EEG, atonic-myoclonic-absence seizures, and clinical photosensitivity. Intellectual disability and/or autism spectrum disorders are common.

According to a specific embodiment, the medical condition is selected from the group consisting of Lennox Gastaut syndrome (LGS), Myoclonic absence epilepsy (MAE), Dravet syndrome, Intellectual disability with epilepsy, Autism spectrum disorder (ASD).

The diagnosis of a CHD2-related neurodevelopmental disorder is established in a proband with a heterozygous CHD2 single-nucleotide pathogenic variant, small indel (insertion/deletion) pathogenic variant, or a partial- or whole-gene deletion detected on molecular genetic testing.

The variation in the CHD2 gene can be a result of a germ-line mutation or de-novo somatic mutation.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology. It will be appreciated that the mammal can also be an embryo or a fetus. Alternatively the subject may be a child or an adolescent up to 15 or 18 years old.

For in vivo therapy, the nucleic acid agent is administered to the subject per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the nucleic acid agent accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include systemic, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, intratumoral or intraocular injections.

According to a specific embodiment, the composition is for inhalation mode of administration.

According to a specific embodiment, the composition is for intranasal administration.

According to a specific embodiment, the composition is for intracerebroventricular administration.

According to a specific embodiment, the composition is for intrathecal administration.

According to a specific embodiment, the composition is for intratumoral administration.

According to a specific embodiment, the composition is for oral administration.

According to a specific embodiment, the composition is for local injection.

According to a specific embodiment, the composition is for systemic administration.

According to a specific embodiment, the composition is for intravenous administration.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the present invention include compositions wherein the active ingredients are contained in an amount effective to achieve the intended purpose. More specifically, a therapeutically effective amount means an amount of active ingredients (e.g. the nucleic acid agent) effective to prevent, alleviate or ameliorate symptoms of a disorder (e.g., associated with CHD2 haploinsufficiency) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide sufficient levels of the active ingredient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Treatment with the nucleic acid agents of the present invention can be augmented with other management protocols known in the art. For example, antiepileptic drugs (AEDs).

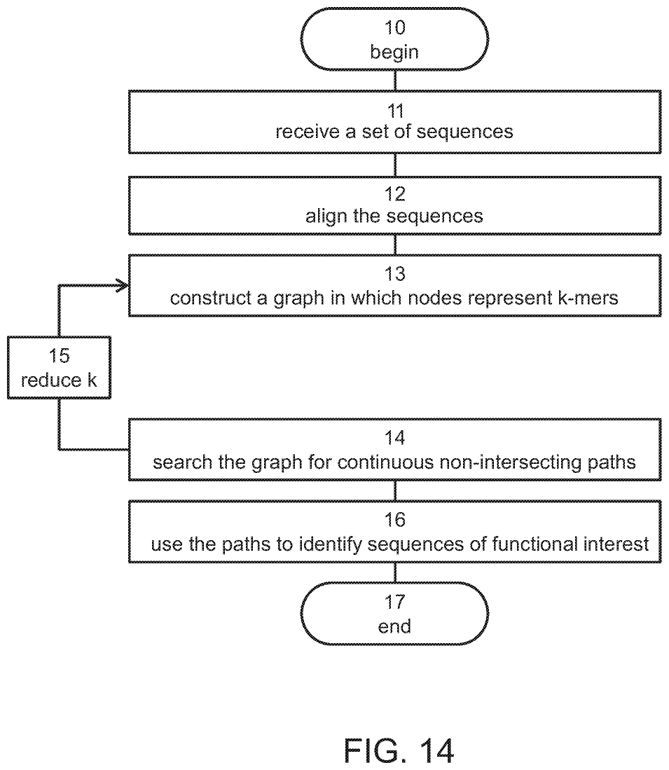

FIG. 14 is a flowchart diagram of a method suitable for analyzing a set of sequences, according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be can be implemented by a data processing system, e.g., a dedicated circuitry or a general purpose computer, configured for receiving data and executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pulls these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

Referring now to FIG. 14, the method begins at 10 and optionally and preferably continues to 11 at which a set of sequences is received. Typically, each sequence in the set describes a polynucleotide, such as, but not limited to, a DNA or an RNA, wherein polynucleotides that are described by different sequences in the set are homologous to each other, as determined manually or using bioinoformatic tools such as Blastn, FASTA and more known to those of skills in the art, as further described hereinbelow and in the Examples section which follows. According to a specific embodiment, the DNA is a genomic DNA. According to another embodiment, the DNA is cDNA or a library DNA. According to a specific embodiment, the DNA represents a locus. According to another embodiment, the DNA is coding or non-coding DNA. According to a specific embodiment, the DNA comprises an exon, an intron or a combination of same. According to a specific embodiment, the sequences are RNA sequences.

According to a specific embodiment, the RNA is a coding RNA. According to another embodiment, the RNA is a non-coding RNA.

In some embodiments of the present invention the homologous polynucleotides are selected from the group consisting of 3′UTR, lncRNA and enhancer.

The polynucleotides in the set can be complete or partial sequences.

In some embodiments of the present invention the method proceeds to 12 at which the sequences in set are aligned according to a predetermined order, e.g., an evolution-dictated, to provide a multiple alignment with multiple alignment layers.

The alignment can be ordered as multiple alignment or using a phylogenetic tree representation-dendogram. Typically, in multiple alignment, the first alignment layer is a sequence that describes a query polynucleotide. When the alignment is evolution-dictated, the first layer is optionally and preferably the sequence that describes the species of interest. For example, when one of the polynucleotides is a human polynucleotide, the first alignment layer can be the sequence of a human polynucleotide.

The alignment can be by any technique known in the art. Typically, the alignment technique provides a score, and the order is according to the score. For example, the order of the sequences can be determined by using BLAST. When the alignment technique provides a score, the second alignment layer is preferably the sequence with the highest alignment score to the first alignment layer, the third alignment layer is preferably the sequence with the next-to-highest alignment score to the first alignment layer, and so on. This provides an alignment in which the sequence in each layer is the one with the best alignment score to the sequence in the preceding layer. In cases in which the alignment technique does not provides a significant alignment to a particular alignment layer, the layer that is subsequent to that particular alignment layer include the next available sequence according to the order of the received set.

It is to be understood, however, that it is not necessary to execute operation 12. For example, the method can use the order as of the received set. Alternatively, the method can allow the user, for example, by a user interface device, to select or input an order to be used by the method.

The method preferably continues to 13 at which a graph is constructed. The Inventors found that it is advantageous to translate the problem of sequence analysis to a problem of traversing a graph since it allows defining the constraints of the problem in a more structured way. The graph is preferably a layered and connected graph, wherein each edge of the graph connects nodes of consecutive layers. The layers of the graph preferably represent the sequences, and the nodes within the layers represent a k-mer within the respective sequences. Thus, for example, suppose that the ith layer of the graph represents a particular sequence of the set (e.g., a sequence of a dog organism). In this case, each node of the ith layer represents a k-mer of the particular sequence. For example, the first node of the ith layer can represent the first k-mer in that particular sequence (e.g., bases 1 through k of the sequence), the second node of the ith layer can represent the second k-mer in that particular sequence (e.g., bases 2 through k+1 of the sequence), and so on. In various exemplary embodiments of the invention 6≤k≤12.

When operation 12 is not executed, and the method does not receive a user input regarding the order, the method constructs the layers of the graph according to the order of the sequences in the received set. Specifically, the first layer of the graph represents the first sequence in the received set, the second layer of the graph represents the second sequence in the received set, and so on. When the method receives a user input regarding the order, the method constructs the layers of the graph according to the user input. Specifically, the first layer of the graph represents the sequence that according to the user input is to be the first in the order, the second layer of the graph represents the sequence that according to the user input is to be the second in the order, and so on. When operation 12 is executed, the method constructs the layers of the graph according to the alignment. Specifically, the first layer of the graph represents the sequence of the first alignment layer, the second layer of the graph represents the sequence of the second alignment layer, and so on.

In various exemplary embodiments of the invention the first layer of the graph represents the sequence that describes the query polynucleotide.

The graph is optionally and preferably constructed such that each edge connects nodes representing identical or homologous k-mers. The advantage of this embodiment is that it allows identifying motifs that are conserved or substantially conserved across multiple polynucleotides.

According to some embodiments of the present invention a homology among homologous k-mers that are connected by an edge of the graph is at least 60%, more preferably at least 70%, more preferably at least 80%, more preferably at least 90%, 95% or more.

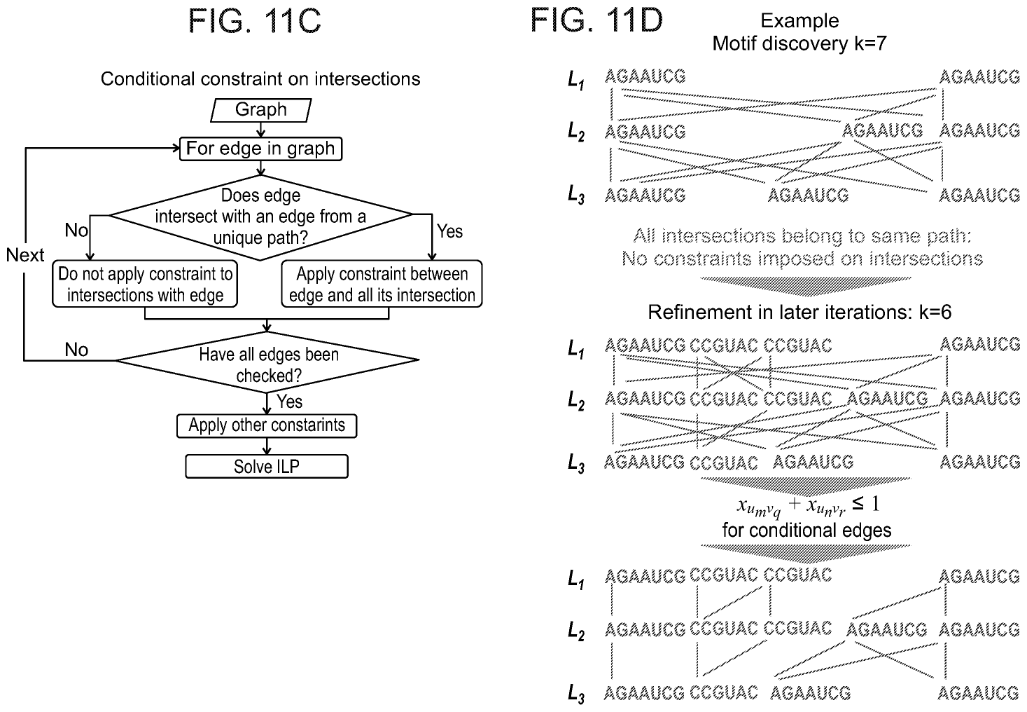

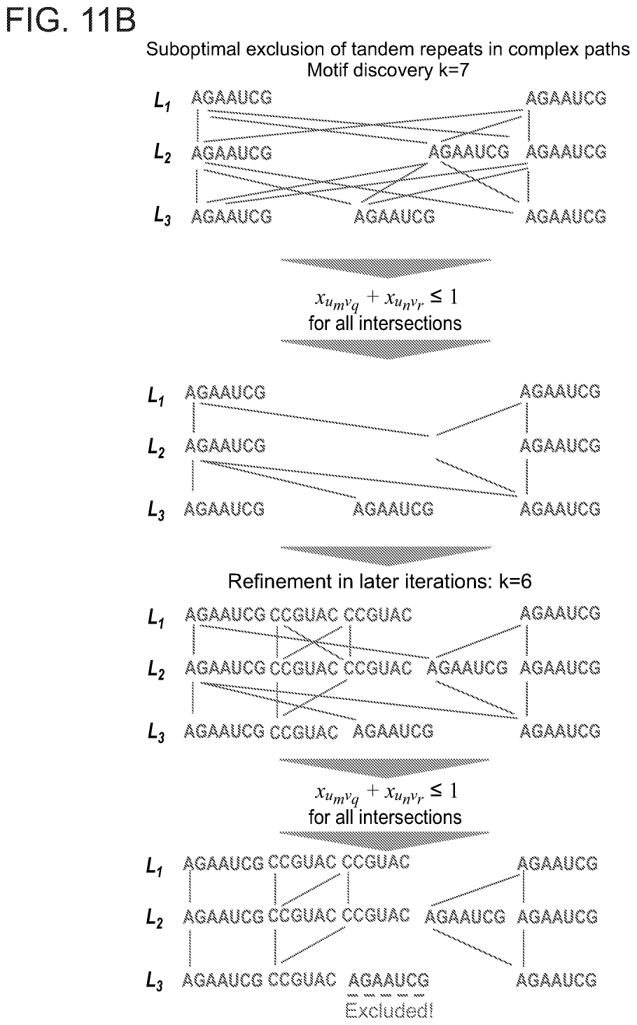

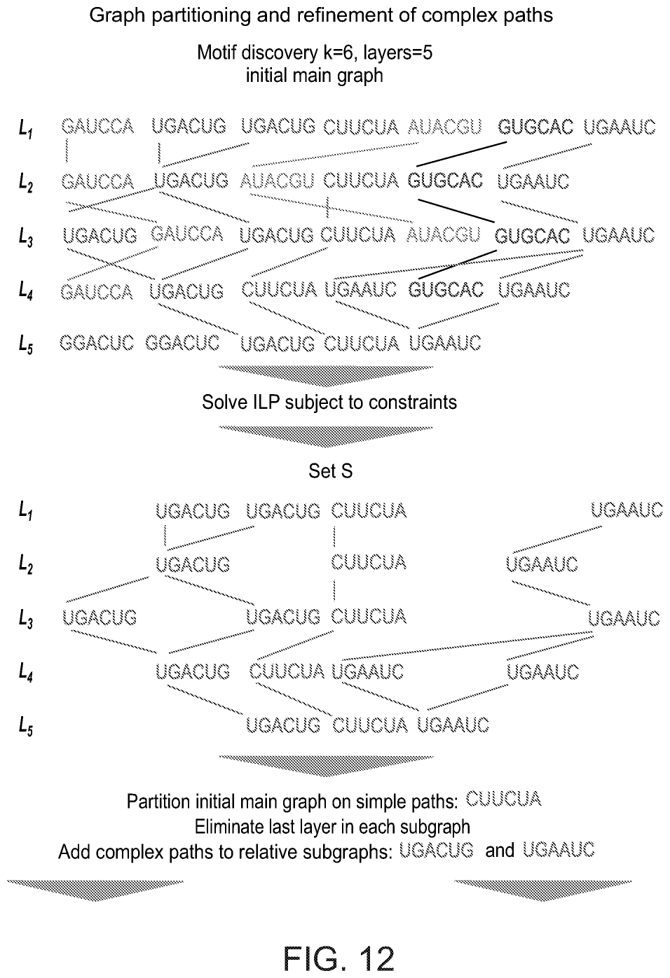

A representative example of typical layered graphs, according to some embodiments of the present invention, is shown in FIGS. 11B, 11D, and 12. In these illustrations, the nodes are shown as strings corresponding to the nucleotide bases that form the k-mers, the edges are shown as straight solid lines, and the layers are denoted L₁, L₂, etc.

The method continues to 14 at which the graph is searched for continuous non-intersecting paths along the edges of the graph. The search can employ any known optimization technique, such as, but not limited to, a linear program (e.g., an Integer Linear Program), a mixed linear program or the like, or any other approach for finding a locally maximal solution, such as a greedy search algorithm.

The paths are non-intersecting in the sense that an edge that connects nodes representing one particular k-mer, does not intersect with any edge that connects nodes representing a k-mer that is not identical or homologous to that particular k-mer. It is noted, however, that when there is more than one edge edges that connects nodes which represent the particular k-mer and which belong to two consecutive layers, these edges may, but not necessarily, intersect. For example, with reference to the simplified graph at the bottom of FIG. 11D, the graph includes two k-mers: eight nodes that represent the 7-mer AGAAUCG, and five nodes that represent the 6-mer CCGUAC. The edges that connects the (identical or homologous) 7-mers do not intersect with the edges that connects the (identical or homologous) 6-mers. On the other hand, there are edges that connect the 7-mers and that intersect each other (see, e.g., the edge that connects the fourth node of layer L₂ with the fourth node of layer L₃, and the edge that connects the fifth node of layer L₂ with the third node of layer L₃). Still, some of the edges that connect the 7-mers do not intersect with any other edge (see, e.g., the edge that connects the fourth node of layer L₂ with the third node of layer L₃, does not intersect with the edge that connects the fifth node of layer L₂ with the fourth node of layer L₃).

In some embodiments of the present invention the search comprises applying a path depth criterion as a constraint for search, such that the search is preferential for deeper paths (namely path that pass through more layers of the graph) than for shallower paths (namely path that pass through less layers of the graph).

From 14 the method optionally and preferably continues to 15 at which the value of k is reduced (preferably by 1) and then loops back to 13 to reconstruct the graph according to the reduced value of k, by including in the graph nodes that represent k-mers that are shorter than the k-mers that are already represented by nodes that already exist in the graph. Preferably, the reconstructions includes adding nodes corresponding to the shorter k-mer, while maintaining at least some of the existing nodes, thus increasing the order (number of nodes) of the graph. Referring again to simplified case in FIG. 11D, the topmost graph in this drawing has eight nodes that represent a 7-mer, and does not include any node that represents a k-mer with k<7. The middle graph in FIG. 11D illustrate a reconstruction of the graph by adding five nodes that represent a 6-mer, so that the order of the graph increases from 8 to 8+5=13.

Once nodes representing shorter k-mers are included in the graph, the method optionally and preferably updates the edges of the graph, so as to connect identical or homologous k-mers of consecutive layers. This is exemplified in the middle graph in FIG. 11D, in which edges were added to the graph to connect the newly added nodes representing 6-mers. The can be added combinatorically, so that any node in layer Lᵢ that represents a particular k-mer is connected to all the nodes in layer Lᵢ+₁ that represent the same particular k-mer.

After each reconstruction of the graph, the method optionally and preferably re-executes operation 14, to provide continuous non-intersecting paths along the edges of the reconstructed graph. Such re-execution may result in exclusion of previously obtained paths, for example, when those previously obtained paths turn out to intersect newly added edges. This is exemplified in the top and graphs of FIG. 11D, where, for example, a path beginning at the leftmost node of layer Lᵢ and ending at the rightmost node of layer L₃ is included in the top graph of FIG. 11D (before the reconstruction) but is not included in the bottom graph in FIG. 11D (after the reconstruction) because it turned out to intersect edges connecting the 6-mers that were added during the reconstruction.

The loopback from 14 to 13 via 15 is optionally and preferably continued in iterative manner. Preferably, at each iteration cycle, the method applies paths obtained in a previous iteration cycle as a constraints for search. A representative example of such application of constraint is illustrated in FIG. 12, and further exemplified in the Examples section that follows. The iteration is optionally and preferably repeated until there are no more k-mers to add, or until there are no more new non-intersecting paths to find or until some other predetermined stop criterion is met.

At 16 an output is generated. The output preferably identifies a k-mer corresponding to at least one of the paths as a nucleic acid sequence of functional interest. The output can be displayed graphically or textually on a display device, or stored in a computer readable storage medium for future use.

The method ends at 17.

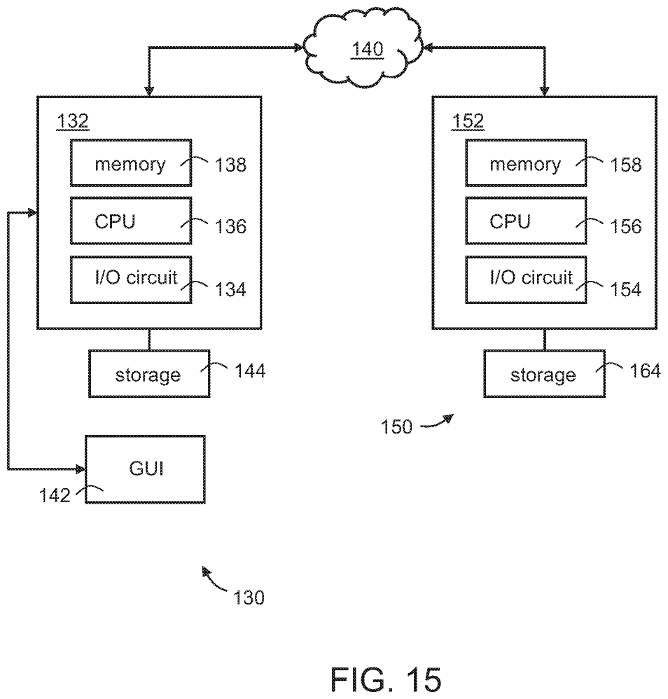

FIG. 15 is a schematic illustration of a client computer 130 having a hardware processor 132, which typically comprises an input/output (I/O) circuit 134, a hardware central processing unit (CPU) 136 (e.g., a hardware microprocessor), and a hardware memory 138 which typically includes both volatile memory and non-volatile memory. CPU 136 is in communication with I/O circuit 134 and memory 138. Client computer 130 preferably comprises a graphical user interface (GUI) 142 in communication with processor 132. I/O circuit 134 preferably communicates information in appropriately structured form to and from GUI 142. Also shown is a server computer 150 which can similarly include a hardware processor 152, an I/O circuit 154, a hardware CPU 156, a hardware memory 158. I/O circuits 134 and 154 of client 130 and server 150 computers can operate as transceivers that communicate information with each other via a wired or wireless communication. For example, client 130 and server 150 computers can communicate via a network 140, such as a local area network (LAN), a wide area network (WAN) or the Internet. Server computer 150 can be in some embodiments be a part of a cloud computing resource of a cloud computing facility in communication with client computer 130 over the network 140.

GUI 142 and processor 132 can be integrated together within the same housing or they can be separate units communicating with each other.

GUI 142 can optionally and preferably be part of a system including a dedicated CPU and I/O circuits (not shown) to allow GUI 142 to communicate with processor 132. Processor 132 issues to GUI 142 graphical and textual output generated by CPU 136. Processor 132 also receives from GUI 142 signals pertaining to control commands generated by GUI 142 in response to user input. GUI 142 can be of any type known in the art, such as, but not limited to, a keyboard and a display, a touch screen, and the like. In preferred embodiments, GUI 142 is a GUI of a mobile device such as a smartphone, a tablet, a smartwatch and the like. When GUI 142 is a GUI of a mobile device, processor 132, the CPU circuit of the mobile device can serve as processor 132 and can execute the code instructions described herein.

Client 130 and server 150 computers can further comprise one or more computer-readable storage media 144, 164, respectively. Media 144 and 164 are preferably non-transitory storage media storing computer code instructions for executing the method as further detailed herein, and processors 132 and 152 execute these code instructions. The code instructions can be run by loading the respective code instructions into the respective execution memories 138 and 158 of the respective processors 132 and 152.

Each of storage media 144 and 164 can store program instructions which, when read by the respective processor, cause the processor to execute the method as described herein. In some embodiments of the present invention, set of sequences describing a plurality of homologous polynucleotides is received by processor 132 by means of I/O circuit 134. Processor 132 constructs a graph, searches the graph for continuous non-intersecting paths, and generates an output identifying a k-mer corresponding to at least one path as a nucleic acid sequence of functional interest, as further detailed hereinabove. Alternatively, processor 132 can transmit the set of sequences over network 140 to server computer 150. Computer 150 receives the set of sequences, constructs a graph, searches the graph for continuous non-intersecting paths, and identifies a k-mer corresponding to at least one path as a nucleic acid sequence of functional interest, as further detailed hereinabove. Computer 150 transmits the nucleic acid sequence of functional interest back to computer 130 over network 140. Computer 130 receives the the nucleic acid sequence and displays it on GUI 142.

Once a motif is identified it can be validated using molecular biology approaches such as by cloning into an expression vector typically with a reporter sequence.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It will be appreciated that RNA antisense sequences may be provided herein as DNA sequences where U is replaced with T.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

### Input to LncLOOM

LncLOOM works on a set of sequences from different species. Typically each sequence corresponds to a putative homolog of a sequence from a different species. Currently, the present inventors work with only one sequence isoform per species, though adaptations to cases where multiple sequences exist per species, e.g., alternative splicing products, are possible. The input sequences are typically constructed through manual inspection of RNA-seq and EST data and existing annotations. It is noted that some of the input sequences might be incomplete, and the present framework, according to some embodiments of the invention, contains specific steps to accommodate such scenarios. Prior to graph building the set is filtered to remove identical sequences. This can be further adjusted by the user to remove sequences with percentage identity above a threshold—in which case LncLOOM uses a MAFFT MSA to compute percentage identity between each pair of sequences, and retain the sequence which appears first in the input dataset.

Sequence Ordering The LncLOOM framework is built around an ordered set of sequences that ideally should be from species with a monotonically increasing evolutionary distance with respect to the anchor sequence (which is human in all the examples in this manuscript). The order of the sequences can be provided by the user, or determined by using BLAST. If BLAST is used, the anchor sequence is defined to be the first sequence in the dataset. The second sequence is the one with the highest alignment score to the anchor sequence. Each subsequent sequence is then the one with the best alignment score to the preceding sequence among the sequences that have not been ordered yet. If no significant alignment is found, the next available sequence in the original input is selected.

### Overview of the LncLOOM Method

Once the ordering of the sequences is established, LncLOOM identifies a set of combinations of short conserved k-mers for different values of k, by reducing each sequence of nucleotides to a sequence of k-mers, each represented by a node in a graph. Identical k-mers in adjacent sequences are connected in the graph, with additional constraints (FIG. 11A-D) and the use of Integer Linear Programming (TLP) to find sets of long non-intersecting paths in these graphs. The set of paths identified in each graph is used to define constraints on graphs in subsequent iterations and to partition the graph (an example of graph partitioning is shown in FIG. 12). Starting with the largest k and iteratively decreasing it, LncLOOM constructs an initial main graph for every k-mer length in a specified range. The main graph is constructed on all ordered sequences in the dataset and is then pruned layer-by-layer (until only the top two sequences remain) into a series of subgraphs for which the ILP problem of each is solved independently. At any given depth, a subgraph may be partitioned into an additional set of smaller subgraphs based on the paths found in previous iterations. In practice, this approach allows us to favor the identification of deeply conserved and longer motifs over shorter and less conserved ones, and to also keep the size of the ILP program to below 1,000 edges, which can be rapidly solved, keeping the overall runtime of LncLOOM to minutes even when applied to dozens of long sequences.

### Graph Building

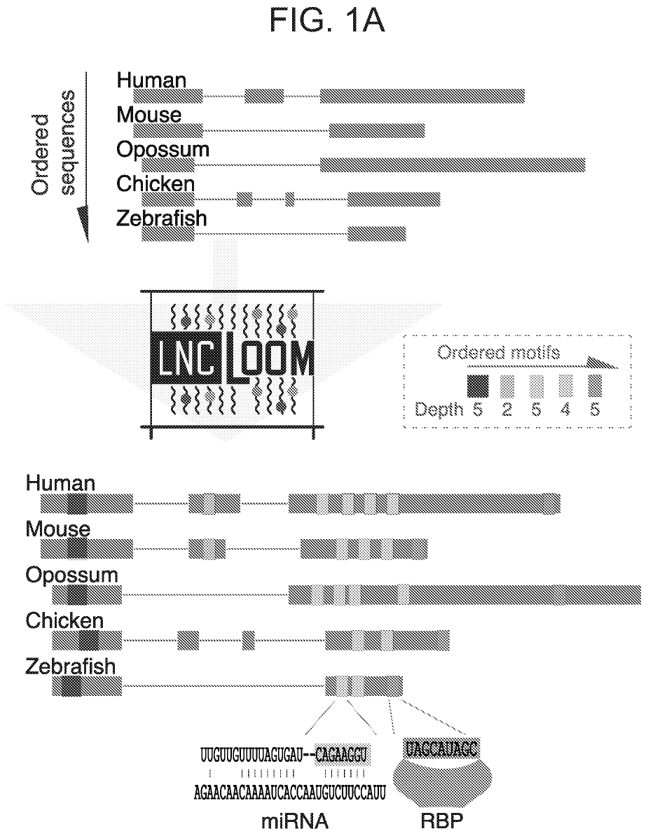

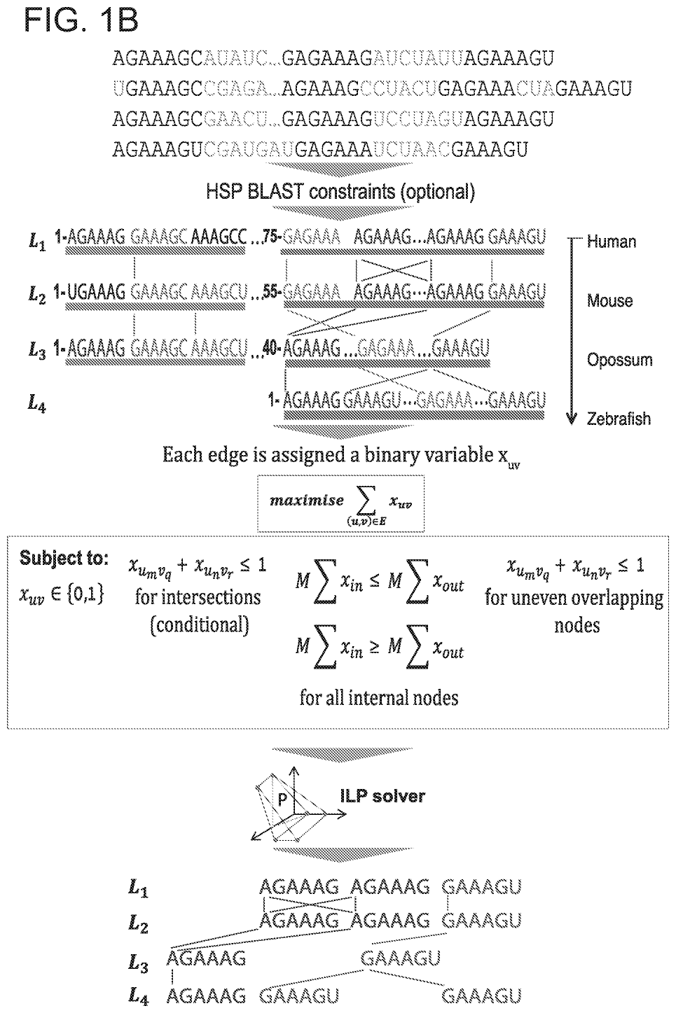

Given a dataset of lncRNA sequences from D species and k-mer length k (6-15 nt), LncLOOM constructs a directed graph G=(V,E), where V is the set of all nodes in the graph and E is the set of edges. The graph is composed of D layers, where D is the number of sequences in the dataset. Each sequence is modelled as a layer (L₁, L₂ . . . L_{D}), and layer Lᵢ, which corresponds to a sequence of length N(f), is composed of nodes (v₁, v₂ . . . v_{N(i)=k+1}) where each node vₙ represents the k-mer at position n in the i-th sequence (FIG. 1B). All pairs of nodes that represent the same k-mer and are found in consecutive layers (Lᵢ and Lⱼ if j=t+1) are connected by an edge xᵤᵥ=(u,v) where [symbol] . Since each substring typically appears multiple times in a sequence, the number of edges may greatly exceed the number of nodes in the graph. Ordered combinations of k-mers that are deeply conserved correspond to long paths in G that do GC not intersect (i.e., for each [symbol] ) and have a node in L₁. A goal is thus to find a set S in E, such that each edge is reachable from L₁ via edges that are in S and no two edges in S intersect. Ideally it is desired to find the largest S, subject to potential additional constraints. For example, short paths may not be desired, and so this requires that edges in S are all found on paths that reach to a certain layer.

### Identification of Long Non-Intersecting Paths Using ILP

In the ILP problem, each edge in G is represented by a variable xᵤᵥ which is assigned a value of 1 if (u,v) is in S. The objective function is defined to maximise |S|:

[symbol]

$$\begin{matrix}maximize {\sum }_{( u , v ) ■ E} {x}_{uv} \\ subject to : \text{?} ∈ ( 0 , 1 )\end{matrix}$$

$$\text{?} \text{indicates text missing or illegible when filed}$$

The additional constraints imposed on this model are derived from several considerations. Firstly, LncLOOM aims to identify short conserved k-mers that appear in the same order in LncRNA sequences. However, it is unlikely that k-mers will appear only once in each sequence. Therefore the constraints applied to the ILP model should allow for complex paths that contain multiple repeats of a single k-mer in one or more layers, provided it is not intersected by a path of a non-matching k-mer that does not have equal depth (FIG. 1B and FIG. 11A). To ensure selection of non-intersecting paths, the following constraint is imposed on any pair of edges that intersect between two consecutive layers:

$$x \text{?} + x \text{?} \leq 1 ,$$

$$\text{?} \text{indicates text missing or illegible when filed}$$

### If;

$$\begin{matrix}m < n and q > r OR m > n and q < r \\ \text{?} , \text{?} ∈ L \text{?} \\ {v}_{q} , {v}_{r} ∈ L {\text{?}}_{■} \\ \text{?} = \text{?} + 1\end{matrix}$$

$$\text{?} \text{indicates text missing or illegible when filed}$$

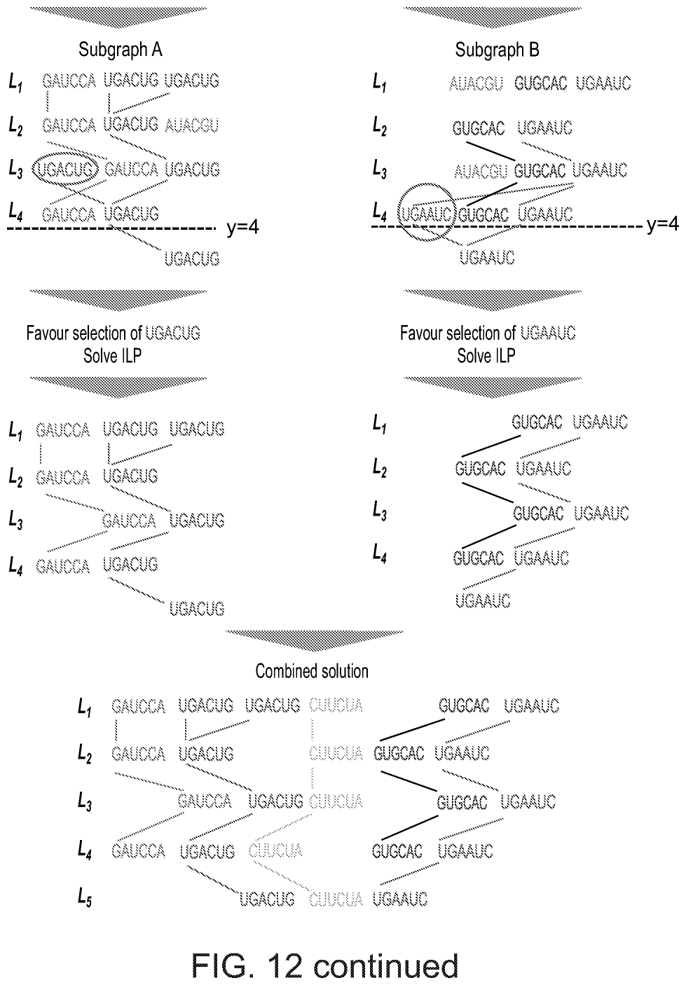

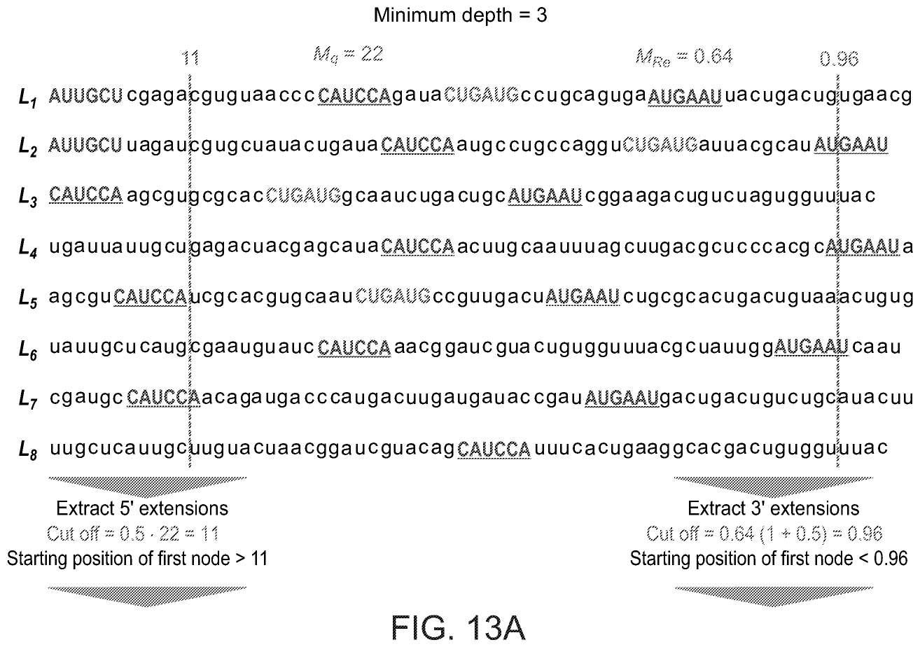

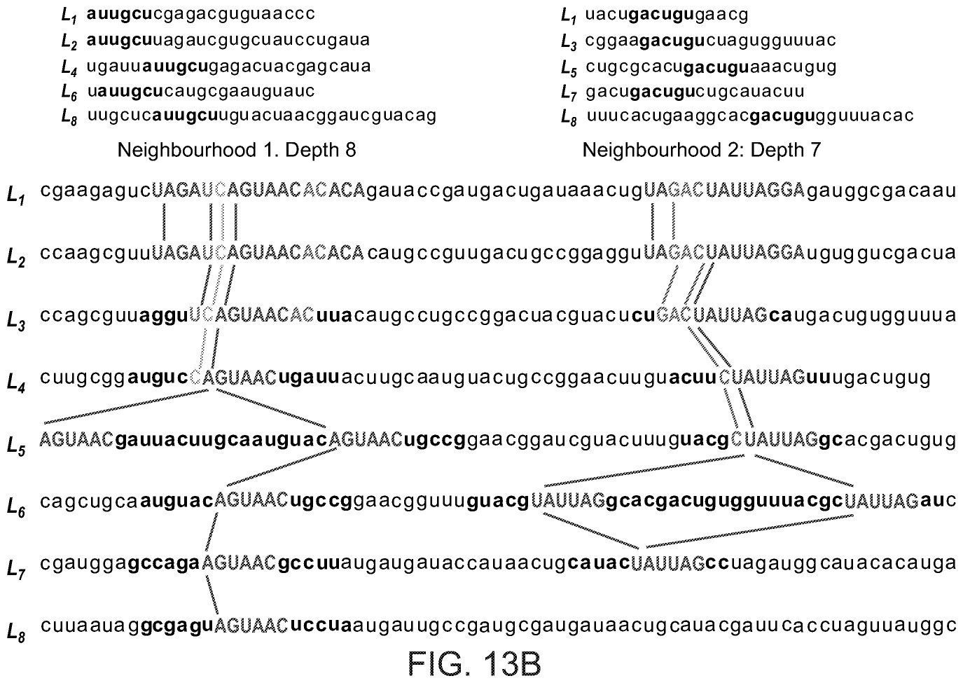

As the above constraint only considers the starting position of each node it also excludes intersecting edges that connect identical k-mers that are repeated in two consecutive layers. In the case where a k-mer is repeated in both consecutive layers, a network of edges is constructed from each repeat-repeat connection (FIG. 11B). This network of edges may override the selection of other paths that are equally conserved but connect fewer k-mers. Therefore it is important to impose this constraint on edges that connect the identical k-mers, as it promotes the splitting of the complex path into multiple non-intersecting paths that are interspersed by paths of uniquely occuring k-mers. However, if the network of edges connecting the identical repeats are constrained only against each other in the absence of any other path, the ILP solver can select any possible solution of edges from the multiple repeat-repeat connections. This can lead to the suboptimal exclusion of repeated k-mers during subsequent iterations of graph refinement (scenario illustrated in FIG. 13B). To avoid this scenario the intersection constraint is only imposed on edges that connect identical k-mers if there is at least one other path, with equal depth, that intersects the network of repeated k-mers.

To favor the selection of deeply conserved k-mers over repetitive shallower k-mers, the following two constraints are imposed on the successors and predecessors of each node

$$\begin{matrix}v ∈ {L}_{t} if 1 < t < y ■ : \\ M {Σ}^{■} \text{?} ■ x \text{?} \leq M {Σ}^{■} \text{?} ■ {x}_{nv} \\ M {Σ}^{■} \text{?} ■ {x}_{nv} \leq M {Σ}^{■} \text{?} ■ x \text{?}\end{matrix}$$

$$\text{?} \text{indicates text missing or illegible when filed}$$

Where Z and P denote the respective subsets of all immediate successors and predecessors of nodex=, y is a minimum depth requirement, and M is a sufficiently large constant (in practice 100 was used). Under this constraint, only paths that have continued connection from L₁ to at least L_{y} are selected. At the same time, this constraint does allow for the selection of connected complex paths that contain tandemly repeated k-mers in one or more layers (FIG. 1B).

In graph G, each layer Lᵢ consists of nodes (v₁, v₂ . . . V_{N(i)-k+1}) that start at every consecutive position in the sequence and have a length of k bases. It follows that from the set S, the set Sᵤₙᵢₒₙ can be formed by merging edges that connect adjacent nodes that overlap with each other. Once the ILP has been solved, these overlapping nodes will be combined into a single longer k-mer. This step may encounter a scenario where a set of adjacent k-mers represent a region of a sequence that contains a string of a single repeated base (see FIG. 1B for an example). It is then possible that layer-specific insertions will be included in the resulting merged k-mer. To overcome this, the following constraint is imposed on any pair of edges that connect adjacent k-mers which overlap in either Lᵢ or Lⱼ such that the start and length of the overlapping region is equal between the two adjacent nodes in each layer:

$$x \text{?} + x \text{?} \leq 1 C$$

$$\text{?} \text{indicates text missing or illegible when filed}$$

### If:

$$\begin{matrix}n \leq m + k - 1 and m < n and ( m + k - 1 ) - n = ( q + k - 1 ) - r \\ OR \\ r \leq q + k - 1 and q < r and ( m + k - 1 ) - n = ( q + k - 1 ) - r \\ \text{?} , \text{?} ∈ {L}_{t} \\ {v}_{q} , {v}_{r} ∈ L {\text{?}}_{■} \\ \text{?} = \text{?} + 1\end{matrix}$$

$$\text{?} \text{indicates text missing or illegible when filed}$$

ILP is a well-known NP-hard problem, which poses a major challenge in the scalability of LncLOOM to very long sequences or large datasets. To overcome this limitation several steps have been included in the framework that reduce the complexity of the ILP of each graph and also favour the selection of deeply conserved k-mers. These include graph pruning, the partitioning of the graph based on simple paths, additional constraints on edge construction and the iterative refinement of non-intersecting complex paths.

Graph Pruning Two pruning steps are used in the LncLOOM framework. The first step involves the exclusion of nodes that correspond to k-mers which are excessively repeated in one or more layers. The number of allowed repeats per layer can be adjusted by the user and can greatly reduce the density of edges in longer sequences when a small k (e.g., 6) is used. For a given k-mer length, this step is performed during the construction of the initial graph on all sequences in the dataset and any excluded nodes are then excluded from all resulting subgraphs. The second pruning step is performed for each iteration of subgraph construction at a given level and excludes all nodes that do not have a connected path from L₁ to the current depth.

### Partitioning the Graph to Reduce Computational Complexity

The constraints imposed on the ILP problem allow for the selection of simple or complex paths, where simple paths are defined as paths that contain only one node per layer. Simple paths consist of definitively selected edges that should not intersect shallower paths and therefore present boundaries at which the graph can be partitioned into smaller subgraphs that can be independently solved (FIG. 12). Currently, these graphs are solved consecutively but in the future there is room for the use of parallel computing to handle larger datasets, provided that at least one simple path is found. The partition is based on simple paths of the current k-mer length that are found at each level in the layer-by-layer iterations. Each subgraph is constructed by selecting a subset of nodes that that is located between two simple paths τₐ and τ_{b} with depth=y, where the boundaries are defined as the ending and starting positions of the nodes within each path: [symbol] for each layer L₁ to L_{y-1} (the last layer is removed for the next iteration). In the case that k-mers of adjacent simple paths overlap, the k-mers are first combined and the boundaries are defined on the starting and ending position of the longer combined k-mer.

### Refinement of Non-Intersecting Complex Paths

In contrast to simple paths, complex paths can contain branches that connect repeated k-mers, particularly in paths that are selected in early iterations when the graph is not constrained. In an unconstrained graph, it is impossible to decipher which of the repeats appear by chance in each layer. Therefore complex paths are not used to constrain edge selection in graphs in subsequent iterations. Instead, the set S that is found in each iteration is divided into: 1) a subset of simple paths that are used for partitioning and edge constraint definition, and 2) a subset of complex paths that are stored separately and continuously refined in the subsequent iterations. During refinement, the complex paths are optimized to remove branches that intersect with newly discovered paths (FIG. 12). The refinement of complex paths is performed at two stages during the layer-by-layer eliminations. Firstly, before solving a subgraph that spans y layers, an individual graph of only complex paths is constructed from the subset [symbol] of longer k-mers with depth=y and the subset [symbol] from paths of the current k-mer length that have a minimum GC depth of y+1 (complex paths selected in previous iterations at the current k-mer length). A subset of refined complex paths, [symbol] is then found according to the ILP problem described above. However, the following additional constraint is imposed to ensure the selection of all complex paths in [symbol] over any shallower path in [symbol] :

For every path τ in

[symbol]

[symbol]

$$\sum {x}_{uv} \geq 1 ∀ ( u , v ) ∈ τ \vert u ∈ {L}_{1} and v ∈ {L}_{2}$$

Under this constraint, at least one repeated k-mer is selected from L₁ for each path τ in [symbol] . When this constraint is imposed together with the constraints described above, a refined path that spans at least layers will be included in the solution. Once the set C_{refined} has been found, the subgraph of all k-mers of the current length and depth is constructed. All paths in C_{refined} are then added to the current subgraph and the ILP problem is solved with the additional constraint imposed to favour the selection of each path τ in C_{refined}. This solution is then divided into a set of simple and complex paths for the next iteration. LncLOOM also includes an option to store and refine simple paths, such that simple paths of shorter k-mers with greater depth are favoured over longer and shallower k-mers. However, if this option is applied the graph is not partitioned and no constraints are imposed on edge construction in subsequent iterations. Therefore, this option is computationally expensive and can only be used to analyse a small dataset of short sequences.

### Using BLAST High Scoring Pairs (HSPs) to Reduce Graph Complexity

BLAST can also be used as an optional step in the process of LncLOOM graph construction. BLAST HSPs are local ungapped alignments between segments, with significant similarity, of sequences found in consecutive layers. The present inventors use these HSPs to constrain edge construction, such that any pair of nodes that are not contained within the same HSP between two consecutive layers are not connected. The HSPs that are found by BLAST are redundant in that HSPs may overlap one another and any segment may be matched to multiple segments in the target sequence. In regard to any set of HSPs that overlap each other, only the most significant pair is included in the HSPs used for graph construction. Similarly, in cases where one segment aligns with multiple segments in the target sequence, only the highest scoring alignment is included. These constraints that are derived from BLAST analysis can effectively decrease the number of possible paths in graphs and promote the correct placement of edges between layers where some of the sequences are incomplete (FIG. 1A).

### Graph Size Restriction

Although steps have been included to reduce the complexity of the ILP problem, in some scenarios the graph is too large to be solved within a reasonable time. To address this bottleneck, the total number of edges in a graph is restricted. By default the maximum number of edges allowed in the ILP problem is 1200, but this can be set to any number above 50. During any iteration, if the number of edges in a graph F exceeds the maximum limit then the graph is divided into a series of subclusters in which the ILP problem is individually solved. Starting with the path that has the fewest edges (fewest repeated k-mers), an individual graph is constructed from each path tin c, and only those paths in [symbol] that intersect it. ILP is then used to optimise the allowed edges in this subcluster of G, [symbol] is then updated to contain these edges and the path τ is removed from G. This process is repeated for each path that remains in G until all paths have been individually optimised against [symbol] or the number of edges in G is the maximum limit, at which point all remaining paths in G are optimised against each other in a single ILP problem. If the number of edges in a graph constructed from an individual subcluster of intersecting paths exceeds the maximum limit then ILP does not proceed and only the paths from [symbol] are retained in the solution.

### Discovery of Motifs in Extended 5′ and 3′ Regions of Sequences

Input to LncLOOM may occasionally contain sequences that are 5′- or 3′-incomplete. As the data set is ordered by homology and not completeness, these sequences may be found in any layer in the graph and obstruct the layer-by-layer connection of nodes in these regions. To reduce the chance that conserved motifs are lost in this scenario, motif discovery is performed in three stages. In the first stage, LncLOOM identifies motifs from a primary graph that is constructed on all sequences in the dataset (a total of D sequences). LncLOOM then determines which sequences have a potentially extended 5′ or 3′ end by considering the position of the first and last motifs in each sequence relative to their median position across all sequences (FIG. 13A). Based on this, LncLOOM builds and solves individual graphs of the extended 5′ and 3′ regions of the more complete sequences in the data set. To build the 5′ extended graph, LncLOOM first calculates the median position, M_{G}, of the starting position of the first node [symbol] in each layer L₁ to L_{D}. A subset of nodes [symbol] is then extracted from each layer [symbol] where τ is some tolerance defined by the user. The nodes of the extended 3′ graph are extracted based on the ending positions of the last motifs relative to the length of each sequence. Specifically, LncLOOM calculates the median relative position, [symbol] , of the ending position of the last node [symbol] each layer L₁ to L_{D}, where

$${Re}_{t} = \frac{{r}_{i} + k - 1}{N ( t )} .$$

A subset of nodes [symbol] is then extracted from each layer [symbol] . By default t=0.5 for the extraction of both the 5′ and 3′ graph but a tolerance can be independently defined for each graph. This step of motif discovery only proceeds if nodes from an extended region of the anchor sequence have been included in the graph. To avoid a scenario where shallowly conserved motifs prevent identification of 5′ or 3′ truncations in deeper layers, for example because of motifs found close to the 5′ end are only conserved in the first two layers, a “minimum depth” parameter can be applied to select the positions of the first and last motif in each sequence from a subset of motifs that are conserved to a specified depth. If the minimum depth parameter is applied then all motifs that do not meet the specified depth requirement are also removed from the solution.

### Calculation of Motif Modules and Neighbourhoods

Once the ILP problem has been solved for all subgraphs in the framework, each set of non-intersecting paths that was selected from the primary, 5′ extended and 3′ extended graphs is processed into motifs modules and neighbourhoods. A motif module is defined as an ordered combination of at least two unique motifs that is conserved in a set of sequences, where each motif is allowed to have any number of tandem repeats. By default, modules are calculated at every layer, [symbol] of the graph by extracting paths that span all layers from L₁ to Lᵢ. If a minimum depth ais specified in the parameters then modules are calculated at every layer [symbol] . As described above, motif discovery is performed through an iterative process of layer-by-layer elimination. This leads to the selection of longer regions of identity as the set of sequences continuously decreases to contain sequences that are more closely related. Consequently, shorter motifs that are more deeply conserved are often embedded in the longer motifs that are only conserved between the top layers (FIG. 13B). The present inventors define these regions within the graph as motif neighbourhoods, where each neighbourhood comprises all nodes in the graph that are connected to a single region of overlapping nodes in L, together with the flanking regions of each node in each layer. To calculate motif neighbourhoods, LncLOOM first combines all overlapping nodes in L₁ to form a set of reference k-mers that represent each neighbourhood. For each reference k-mer, all paths that are connected to each shorter k-mer which is embedded within the reference k-mer are then included into that neighbourhood. For each motif in each layer, the length of flanking regions is calculated relative to the position of the motif in the reference k-mer (FIG. 13B). The motifs modules and neighbourhoods from each of the primary, 5′ extended and 3′ extended graphs are presented in HTML and plain text file formats.

### Calculation of Motif Significance

Motif significance is inferred by calculating empirical p-values of each motif in two genres of random datasets. Firstly, for a motif of length k that is conserved to L, the present inventors determine the empirical probability of finding the exact motif found in the real dataset and any combination of the same number of any motifs of the same length or greater at least once in L of a set of random sequences that has the same percentage identity between consecutive layers as observed in the input sequences. This is achieved by using MAFFT to generate an MSA of the input sequences, and then running multiple iterations of LncLOOM (100 for the analyses described in this manuscript) iterations in which the columns of the MSA are randomly shuffled. Secondly, the present inventors determine the empirical probability of finding the exact motif and any combination of the same number of any motifs of the same length at least once in Lᵢ of a set of random sequences generated such that each layer has the same length and the same dinucleotide composition of its corresponding layer in the input sequences (but without preserving % identity between layers). Only the former P-values were used in the analyses described in this manuscript. Multiprocessing has been implemented to execute the iterations in parallel.

### Functional Annotation of Motifs

LncLOOM has two optional annotation features. Firstly, the discovered motifs can be mapped to binding sites of miRNAs by identifying perfect base pairing with the seed regions of conserved (conserved throughout mammals) and broadly conserved (typically found throughout vertebrates) miRNAs from TargetScan. For each motif, the type of pairing (6mer, 7mer, 7mer-A1, 7mer-M8 or 8mer) is determined in each sequence by considering the motif together with the immediate flanking base from both sides of the motif. A match is only found if the complete seed region (6mer) directly matches the motif. Secondly, motifs that are found in genes that are expressed in HepG2 or K562 cell lines can also be mapped to binding sites of RBPs identified by eCLIP in the ENCODE project. To determine the chromosome coordinates of each motif in a selected query sequence, LncLOOM uses BLAT (Kent, 2002) to align the sequence to the genome and then calculates overlaps with the coordinates of binding sites of RBPs which are extracted from ENCODE bigBed files using the pyBigWig package. Alternatively, the user can also upload a bed file that specifies the chromosome coordinates and length of each exon in the query sequence. The extracted eCLIP data is filtered to exclude all peaks with enrichment <2 over the mock input. RBPs that bind a large portion of the anchor sequence are marked, as the overlap of their binding peaks with any conserved motif is less likely to be functionally relevant for that specific motif.

### LncLOOM Implementation and Availability

Graph building is performed using the networkx package. The integer programming problems are modelled using PuLP and are solved by either the open source COIN-OR Branch-and-Cut solver (CBC) (www(dot)coin-or(dot)org/) or the commercial Gurobi solver (www(dot)gurobi(dot)com/). LncLOOM utilizes the following alignment programs during graph construction, motif annotation and the empirical evaluation of motif significance: BLAST, BLAT and MAFFT. The multiprocessing python package is used to compute statistical iterations in parallel.

### Calculation of Motif Enrichment

For evaluating the enrichment of specific motifs in sequences, the present inventors generated 1,000 sets of random sequences matching the dinucleotide composition of the input sequences and counted the occurrences of the motifs to compute the expected number of motifs and the empirical p-values.

LncLOOM Analysis of lncRNAs and 3′UTRs

LncLOOM was used to analyse Cyrano sequences from 18 species, libra (Nrep in mammals) from 8 species, Chaserr sequences from 16 species, DICER1 sequences from 12 species and a PUM1 and PUM2 sequences from 16 species. For all genes, LncLOOM parameters were set to search for k-mers from 15 to 6 bases in length and the sequences were reordered by BLAST with the Human sequence defined as the anchor sequence in each case. HSPs constraints were not imposed. Motif significance was calculated over 100 iterations. The order of sequences for each gene as represensent in the LncLOOM framework is shown in Table 1.

LncLOOM was also used to analyse 2,439 3′UTR genes. The datasets were constructed from 3′UTR MSAs generated by TargetScan7.2 miRNA target site prediction suite¹⁰ and included the sequences of human, mouse, dog, and chicken that were between 300 and 3,000 nt. Depending on availability and length (>200 bases), sequences from frog, shark, zebrafish, gar and lamprey, cioan and fly were obtained from Ensembl and added to their respective gene datasets. For each dataset BLASTN is used, with a cutoff E-value of 0.05, to classify which sequences in each of the respective species had no detectable alignment to their human ortholog, as well as those sequences that also did not align to mouse, dog and chicken. K-mers identified by LncLOOM were matched to seeds of broadly conserved miRNA families, for which TargetScanHuman reported a hsa-miRNA. To evaluate the sensitivity of LncLOOM, the broadly conserved miRNA binding sites identified by LncLOOM were compared to predictions reported by TargetScan (www(dot)targetscan(dot)org/cgi-bin/targetscan/data_download.vert72.cgi). Specifically, the present inventors only compared the miRNA sites from genes in which TargetScan reported sites in the identical representative human transcript as used in the present LncLOOM datasets. In total this corresponded to 2,359 of the 2,439 genes.

### Tissue Culture

Neuro2a cells (ATCC) were routinely cultured in DMEM containing 10% fetal bovine serum and 100 U penicillin/0.1 mg ml⁻¹ streptomycin at 37° C. in a humidified incubator with 5% CO₂. Cells were routinely tested for mycoplasma contamination and were not authenticated.

### Mass Spectrometry Sample Preparation

Samples were subjected to in-solution tryptic digestion using suspension trapping (S-trap) as previously described⁴⁷. Briefly, after pull-down proteins were eluted from the beads using 5% SDS in 50 mM Tris-HCl. Eluted proteins were reduced with 5 mM dithiothreitol and alkylated with 10 mM iodoacetamide in the dark. Each sample was loaded onto S-Trap microcolumns (Protifi, USA) according to the manufacturer's instructions. After loading, samples were washed with 90:10% methanol/50 mM ammonium bicarbonate. Samples were then digested with trypsin for 1.5 h at 47° C. The digested peptides were eluted using 50 mM ammonium bicarbonate. Trypsin was added to this fraction and incubated overnight at 37° C. Two more elutions were made using 0.2% formic acid and 0.2% formic acid in 50% acetonitrile. The three elutions were pooled together and vacuum-centrifuged to dryness. Samples were kept at −80° C. until further analysis.

### Liquid Chromatography

ULC/MS grade solvents were used for all chromatographic steps. Dry digested samples were dissolved in 97:3% H₂O/acetonitrile+0.1% formic acid. Each sample was loaded using split-less nano-Ultra Performance Liquid Chromatography (10 kpsi nanoAcquity; Waters, Milford, MA, USA). The mobile phase was: A) H₂O+0.1% formic acid and B) acetonitrile+0.1% formic acid. Desalting of the samples was performed online using a reversed-phase Symmetry C18 trapping column (180 μm internal diameter, 20 mm length, 5 μm particle size; Waters). The peptides were then separated using a T3 HSS nano-column (75 μm internal diameter, 250 mm length, 1.8 μm particle size; Waters) at 0.35 μL/min. Peptides were eluted from the column into the mass spectrometer using the following gradient: 4% to 30% B in 55 min, 30% to 90% B in 5 min, maintained at 90% for 5 min and then back to initial conditions.

### Mass Spectrometry

The nanoUPLC was coupled online through a nanoESI emitter (10 m tip; New Objective; Woburn, MA, USA) to a quadrupole orbitrap mass spectrometer (Q Exactive HF, Thermo Scientific) using a FlexIon nanospray apparatus (Proxeon).

Data was acquired in data dependent acquisition (DDA) mode, using a Top10 method. MS1 resolution was set to 120,000 (at 200 m/z), mass range of 375-1650 m/z, AGC of 3e6 and maximum injection time was set to 60 msec. MS2 resolution was set to 15,000, quadrupole isolation 1.7 m/z, AGC of le5, dynamic exclusion of 20 sec and maximum injection time of 60 msec.

### Mass Spectrometry Data Processing and Analysis

Raw data was processed with MaxQuant v1.6.6.0. The data was searched with the Andromeda search engine against the mouse (Mus musculus) protein database as downloaded from Uniprot (www(dot)uniprot(dot)com), and appended with common lab protein contaminants. Enzyme specificity was set to trypsin and up to two missed cleavages were allowed. Fixed modification was set to carbamidomethylation of cysteines and variable modifications were set to oxidation of methionines, and protein N-terminal acetylation. Peptide precursor ions were searched with a maximum mass deviation of 4.5 ppm and fragment ions with a maximum mass deviation of 20 ppm. Peptide and protein identifications were filtered at an FDR of 1% using the decoy database strategy (MaxQuant's “Revert” module). The minimal peptide length was 7 amino-acids and the minimum Andromeda score for modified peptides was 40. Peptide identifications were propagated across samples using the match-between-runs option checked. Searches were performed with the label-free quantification option selected. The quantitative comparisons were calculated using Perseus v1.6.0.7. Decoy hits were filtered out. A Student's t-Test, after logarithmic transformation, was used to identify significant differences between the experimental groups, across the biological replica. Fold changes were calculated based on the ratio of geometric means of the different experimental groups.

### RNA-Pulldown Assay

Templates for in vitro transcription were generated by amplifying synthetic oligos (Twist Bioscience) and adding the T7 promoter to the 5′ end for sense sequences and to the 3′ end for antisense control sequences (see Table 2 for full sequences). Biotinylated transcripts were produced using the MEGAscript T7 in vitro transcription reaction kit (Ambion) and Biotin RNA labeling mix (Roche). Template DNA was removed by treatment with DNaseI (Quanta). Neuro2a cells (ATCC) were lysed with RIPA supplemented with protease inhibitor cocktail (Sigma-Aldrich, #P8340)+100 U/ml RNase inhibitor (#E4210-01), and 1 mM DTT for 15 min on ice. The lysate was cleared by centrifugation at 21130×g for 20 min at 4° C. Streptavidin Magnetic Beads (NEB #S1420S) were washed twice in buffer A(NaOH 0.1M and NaCl 0.05M), once in buffer B (NaCl 0.05M) and then resuspended in two tubes of binding/washing (NaCl 1M, 5 mM Tris-HCl pH 7.5 and 0.5 mM EDTA supplement with PI+100 U/ml RNase inhibitor, and 1 mM DTT). One tube of beads was washed three times in RIPA supplemented with PI and DTT 1 mM, after which cell lysate was added and pre-cleared with overhead rotation at 4° C. for 30 min. The second tube was equally divided into individual tubes for each RNA probe. 2-10 pmol of the biotinylated transcripts were then added to the respective tubes and rotated overhead at 4° C. for 30 min. The beads were then washed three times in binding/washing buffer, afterwhich equal amounts of the pre-cleared cell lysate was added to each sample of beads and RNA probe. The samples were then rotated overhead at 4° C. for 30 min. Following rotation, the beads were washed three times with high salt CEB (10 mM HEPES pH7.5, 3 mM MgCl₂, 250 mM NaCl, 1 mM DTT and 10% glycerol). Proteins were then eluted from the beads in 5% SDS in 50 mM Tris pH 7.4 for 10 min in room temperature.

### Antisense Oligonucleotide and LNA GapmeR Transfections

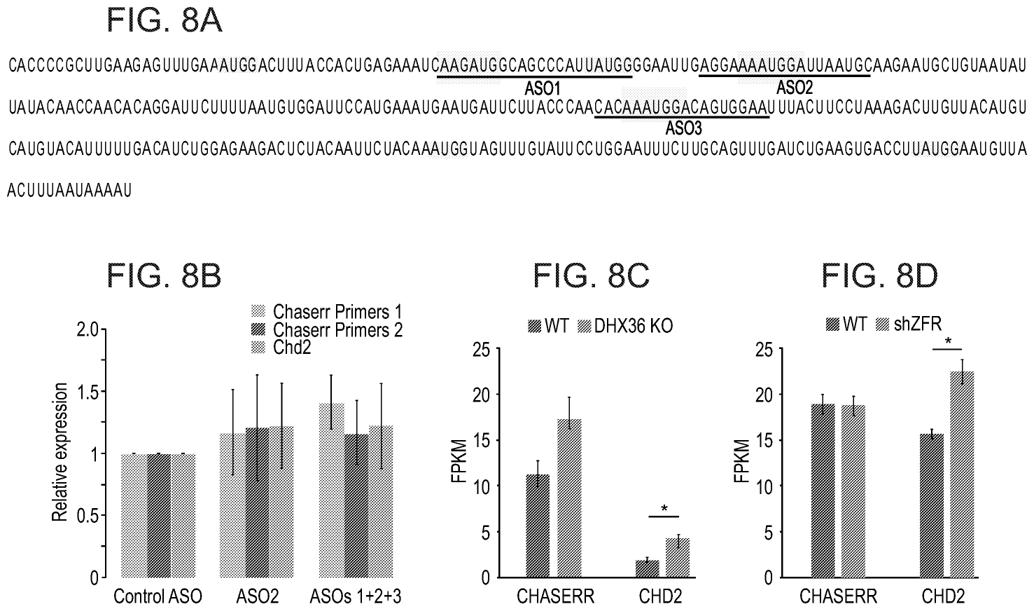

ASOs (Integrated DNA Technologies) were designed to target the conserved ATGG sites that were identified by LncLOOM in the last exon of mouse Chaserr (FIG. 8A). All ASOs were modified with 2′-O-methoxy-ethyl bases. LNA gapmers (Qiagen), targeted to Chaserr introns, were used for Chaserr knockdown (see Table 3 for full oligo sequences). Transfection: 2×10⁵ Neuro2A cells were seeded in a six-well plate and transfected by using Lipofectamine 3000 (Life Technologies, L3000-008) following the manufacturer's protocol with a mix of LNA1-4 or with ASO1, ASO2, ASO3, or a mix of either ASO1 and ASO3 or ASO1-3 to a final concentration of 25 nM. Endpoints for all experiments were at 48 hr post transfection, after which the cells were collected with TRIZOL for RNA extraction and assessment by RT-qPCR analysis.

### RNA Immunoprecipitation (RIP)

Neuro2a cells (ATCC) were collected, centrifuged at 94×g for 5 min at 4° C., and washed twice with ice-cold phosphate-buffered saline (PBS) supplemented with ribonuclease inhibitor (100 U/mL, #E4210-01) and protease inhibitor cocktail (Sigma-Aldrich, #P8340). Next, cells were lysed in 1 mL of lysis buffer (5 mM PIPES, 200 mM KCl, 1 mM CaCl₂, 1.5 mM MgCl₂, 5% sucrose, 0.5% NP-40, supplemented with protease inhibitor cocktail+100 U/ml RNase inhibitor, and 1 mM DTT) for 10 min on ice. Lysates were sonicated (Vibra-cell VCX-130) three times for 1 s ON, 30 s OFF at 30% amplitude, followed by centrifugation at 21130×g for 10 min at 4° C. Supernatants were then transferred to new 2-mL tubes and supplemented with 1 mL of IP binding/washing buffer (150 mM KCl, 25 mM Tris (pH 7.5), 5 mM EDTA, 0.5% NP-40, supplemented with protease inhibitor cocktail+100 U/ml RNase inhibitor, and 0.25 mM DTT). The samples were then rotated for 2-4 hr at 4° C. with 5 μg of antibody per reaction. 50 μl of beads GenScript A/G beads (#L00277) per reaction were washed three times with IP binding/washing buffer, followed by addition to lysates for an overnight rotating incubation. After incubation, the beads were washed three times inTP binding/washing buffer. 10% of each sample was collected and boiled for 5 min at 95° C. for further analysis by western blot. The remaining beads were resuspended in 0.5 mL of TRIZOL for RNA extraction and assessment by RT-qPCR analysis where immunoprecipitation material was normalized to total cell lysate.

### Western Blot

Protein samples collected from RIP were resolved on 8-10% SDS-PAGE gels and transferred to a polyvinylidene difluoride (PVDF) membrane. After blocking with 5% nonfat milk in PBS with 0.1% Tween-20 (PBST), the membranes were incubated with the primary antibody followed by the secondary antibody conjugated with horseradish peroxidase. Blots were quantified with Image Lab software. The primary antibody anti-Dhx36 (Bethyl, #A300-525A, 1:1,000 dilution) and secondary antibody anti-rabbit (JIR #111-035, 1:10,000 dilution) were used.

qRT-PCR

Total RNA was extracted from transfected N2a cells using TRIREAGENT (MRC) according to the manufacturer's protocol. cDNA was synthesized using qScript Flex cDNA synthesis kit (95049, Quanta) with random primers. Fast SYBR Green master mix (4385614) was used for qPCR. Gene expression levels were normalised to the housekeeping genes Actin and Gapdh.

TABLE 1
Order of sequences analysed by LncLOOM.

| Layer | Cyrano | libra | Chaserr | DICER1 | PUM1 | PUM2 |
|---|---|---|---|---|---|---|
| 1 | Human | Human | Human | Human | Human | Human |
| 2 | Rhesus | Dog | Dog | Cow | Dog | Dog |
| 3 | Cow | Mouse | Ferret | Dog | Cow | Cow |
| 4 | Dog | Opossum | Pig | Opossum | Opossum | Mouse |
| 5 | Rabbit | Chicken | Rabbit | Xenopus | Chicken | Chicken |
| 6 | Rat | Xenopus | Armadillo | Zebrafish | Lizzard | Lizzard |
| 7 | Mouse | Spotted | Mouse | Medaka | Mouse | Shark |
| | | Gar | | | | |
| 8 | Opossum | Zebrafish | Opossum | Mouse | Zebrafish | Opossum |
| 9 | Chicken | | Platypus | Lancelet | Tetraodon | Xenopus |
| 10 | Xenopus | | Lizard | Sea Urchin | Stickleback | Tetraodon |
| 11 | Spotted Gar | | Chicken | Fly | Xenopus | Stickleback |
| | | | | (DICER1) | | |
| 12 | Nile Tilapia | | Nile | Fly | Shark | Zebrafish |
| | | | Tilapia | (DICER2) | | |
| 13 | Fugu | | Stickleback | | Lamprey | Lamprey |
| 14 | Medaka | | Medaka | | Lancelet | Lancelet |
| 15 | Stickleback | | Zebrafish | | Ciona | Ciona |
| 16 | Atlantic Cod | | Xenopus | | Fly | Fly |
| 17 | Zebrafish | | | | | |
| 18 | Elephant | | | | | |
| | Shark | | | | | |

TABLE 2
Oligonucleotide sequences used for RNA pulldown.
Mutated bases are underlined

| Oligo | | Sequence |
|---|---|---|
| name | Description | (SEQ ID NO: 88-90) |
| Exon5- | WT sequence | Caccccgcttgaagagtttgaaatg |
| WT | of Mouse | gactttaccactgagaaatcaagat |
| | Chaserr | ggcagcccattatggggaattgagg |
| | Exon 5 | aaaatggattaatgcaagaatgctg |
| | | taatattatacaaccaacacaggat |
| | | tcttttaatgtggattccatgaaat |
| | | gaatgattcttacccaacacaaatg |
| | | gacagtggaatttacttcctaaaga |
| | | cttgttacatgtcatgtacattttt |
| | | gacatctggagaagactctacaatt |
| | | ctacaaatggtagtttgtattcctg |
| | | gaatttcttgcagtttgatctgaag |
| | | tgaccttatggaatgttaactttaa |
| | | taaaat |
| Exon5- | Mouse Chaserr | Caccccgcttgaagagtttgaaatg |
| MC | Exon 5 | gactttaccactgagaaatcaagTA |
| | with four | CCcagcccattTACCggaattgagg |
| | ATGG->TACC | aaaTACCattaatgcaagaatgctg |
| | mutations. | taatattatacaaccaacacaggat |
| | All four | tcttttaatgtggattccatgaaat |
| | are located | gaatgattcttacccaacacaaTAC |
| | within | Cacagtggaatttacttcctaaaga |
| | conserved | cttgttacatgtcatgtacattttt |
| | motif | gacatctggagaagactctacaatt |
| | identified by | ctacaaatggtagtttgtattcctg |
| | LncLOOM | gaatttcttgcagtttgatctgaag |
| | | tgaccttatggaatgttaactttaa |
| | | taaaat |
| Exon5- | Mouse Chaserr | CaccccgcttgaagagtttgaaTAC |
| MA | Exon 5 | CactttaccactgagaaatcaagTA |
| | with all | CCcagcccattTACCggaattgagg |
| | ATGG sites | aaaTACCattaatgcaagaatgctg |
| | mutated | taatattatacaaccaacacaggat |
| | to TACC. | tcttttaatgtggattccatgaaat |
| | In total 7 | gaatgattcttacccaacacaaTAC |
| | ATGG->TACC | Cacagtggaatttacttcctaaaga |
| | mutations. | cttgttacatgtcatgtacattttt |
| | | gacatctggagaagactctacaatt |
| | | ctacaaTACCtagtttgtattcctg |
| | | gaatttcttgcagtttgatctgaag |
| | | tgaccttTACCaatgttaactttaa |
| | | taaaat |

TABLE 3
Oligonucleotide sequences of
ASOs and LNA GapmeRs

| | Sequence |
|---|---|
| Name | (SEQ ID NO: 91-99) |
| ASO NTC | CTCTCTCTCTTTCTAT |
| (Control ASO) | CCCTTC |
| ASO1 | CCATAATGGGCTGCCATCTT |
| ASO2 | GCATTAATCCATTTTCCT |
| ASO3 | TTCCACTGTCCATTTGTG |
| LNA NTC | AACACGTCTATACGC |
| (Control GapmeR) | (Cat#: LG00000002) |
| LNA1 | ATAGCGTGCATAAATT |
| LNA2 | GCAGAATGAAGACAAA |
| LNA3 | ATCAATGAATTCACAT |
| LNA4 | CAACGACTGATCCTAA |

TABLE 4
Primer sequences

| Gene | Forward primer (SEQ ID NO) | Reverse primer/(SEQ ID NO) |
|---|---|---|
| Chaserr (Primer 1) | GCCATTTTGAAGACTGAGACC | TCTATGGTGCAGGCCTT |
| | A/100 | TCA/101 |
| Chaserr (Primer 2) | TGACATCTGGAGAAGACTCTAC | AGGTCACTTCAGATCAAA |
| | AA/102 | CTGC/103 |
| Chd2 | GGAGATCATAGAACGGGCCA/104 | AAAAGGGTTTGAGTTGGA |
| | | TCTTC/105 |
| Actin | TTGGGTATGGAATCCTGTGG/106 | CTTCTGCATCCTGTCAG |
| | | CAA/107 |
| Gapdh | GTCGGTGTGAACGGATTTG/108 | GAATTTGCCGTGAGTGG |
| | | AGT/109 |
| Malat1 | GTTACCAGCCCAAACCTCAA/110 | CACTTGTGGGGAGACCTT |
| | | GT/111 |
| For amplification | TAATACGACTCACTATAGGGC | AAGTTAACATTCCATAAG |
| of Exon5_WT and | ACCCCGCTTGAAGAG/112 | GTCACTTCAG/113 |
| Exon5_MC for T7 | | |
| in vitro | | |
| transcription | | |
| For amplification | TAATACGACTCACTATAGGGAA | CACCCCGCTTGAAGAG/115 |
| of Exon5_WT and | GTTAACATTCCATAAGGTCACT | |
| Exon5_MC | TCAG/114 | |
| Antisense for T7 in | | |
| vitro transcription | | |
| For amplification | TAATACGACTCACTATAGGGC | AAGTTAACATTGGTAAAG |
| of Exon5_MA for | ACCCCGCTTGAAGAG/116 | GTCACTTCAG/117 |
| T7 in vitro | | |
| transcription | | |
| For amplification | TAATACGACTCACTATAGGGAA | CACCCCGCTTGAAGAG/119 |
| of Exon5_MA | GTTAACATTGGTAAAGGTCACT | |
| Antisense for T7 in | TCAG/118 | |
| vitro transcription | | |

### EXAMPLE 1

### The LncLOOM Framework

LncLOOM receives a collection of putatively homologous sequences of a genomic sequence of interest. An embodiment focuses on lncRNAs and 3′UTRs, but other elements, such as enhancers, can be readily used as well. For lncRNAs only the exonic sequences are used for motif identification, but LncLOOM visualizes the positions of the exon-exon junctions. The input sequences are provided in a certain order (FIG. 1A), which ideally concurs with the evolutionary distances between the species, and which can be set automatically based on sequence similarity. The precise definitions of the data structures and algorithms used in LncLOOM appear in Materials and Methods, and an overview of the framework is presented in FIGS. 1A-B. LncLOOM represents each RNA sequence as a ‘layer’ of nodes in a network graph (FIG. 1B), where each node represents a short k-mer (e.g., k between 6 and 15). The order of the layers reflects the evolutionary distance of input sequences from a query sequence, which is placed in the first layer of the graph (human in the analyses described here), and sequences from the other species are placed in additional sequential layers of the graph. Edges in the graph connect between nodes with identical k-mers in consecutive layers. It will be appreciated that it is possible to also connect ‘similar’ k-mers. Under these definitions, an objective is to identify combinations of long ‘paths’ in the graph that do not intersect each other and therefore connect short motifs that maintain the same order in different sequences. As the interest is typically in motifs that are present in the top layer, it is a requisite that paths begin in it. The problem of identifying the maximal set of such paths is computationally hard, since for k=1 it is the same as the longest common subsequence problem, but present results show that it can be translated into a problem of solving an Integer Linear Program (ILP), for which it is computationally hard to find an optimal solution, but efficient solvers are available (FIG. 1B and Methods).

Once the graph is constructed, the process begins with identifying paths for the largest k value, and then use these paths (if found) to constrain the possible locations of paths for smaller k. This approach allows to favor longer conserved elements but also to identify significantly conserved short k-mers. Once all k values are tested, the resulting graphs are merged to obtain a combination of the motifs and the depths to which they are conserved. In order to compute the statistical significance of the motif conservation, an MSA of the input sequences is generated, the alignment columns are shuffled so as to derive random sequences with an internal similarity structure similar to that of the input sequences. The full LncLOOM pipeline is then applied to these sequences, and for each motif found in the original input sequences to be conserved to layer D, the empirical probability of identifying either precisely the same motif, or a combination of the same number of any motifs of that length, conserved to layer D. Additional P-values are computed for a less stringent control, where random sequences with the same dinucleotide composition are generated and the inter-sequence similarity structure is not preserved.

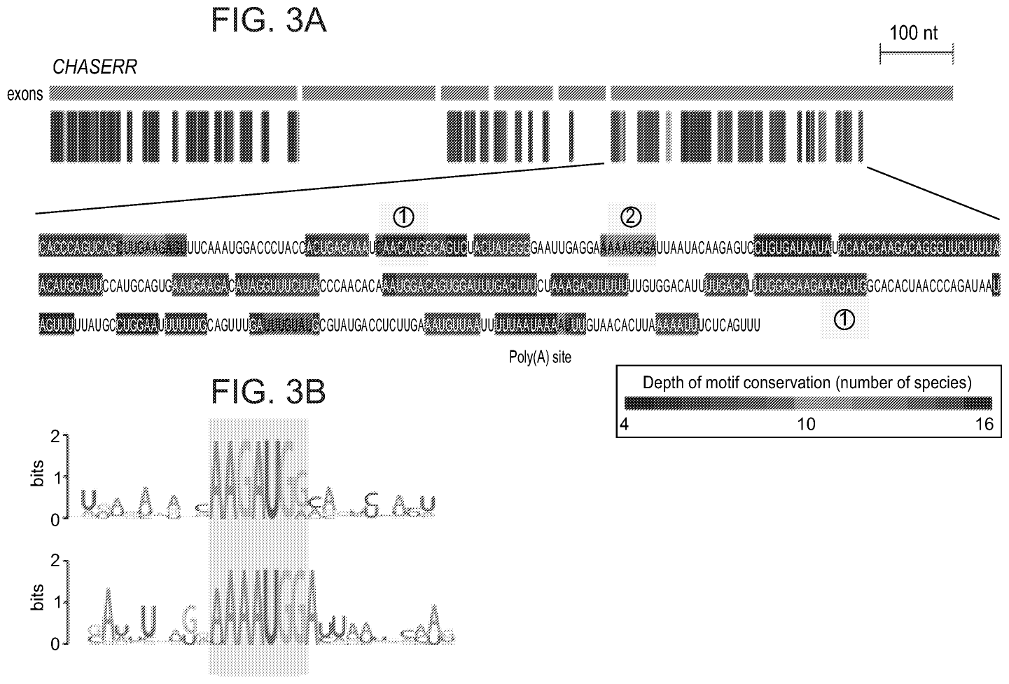

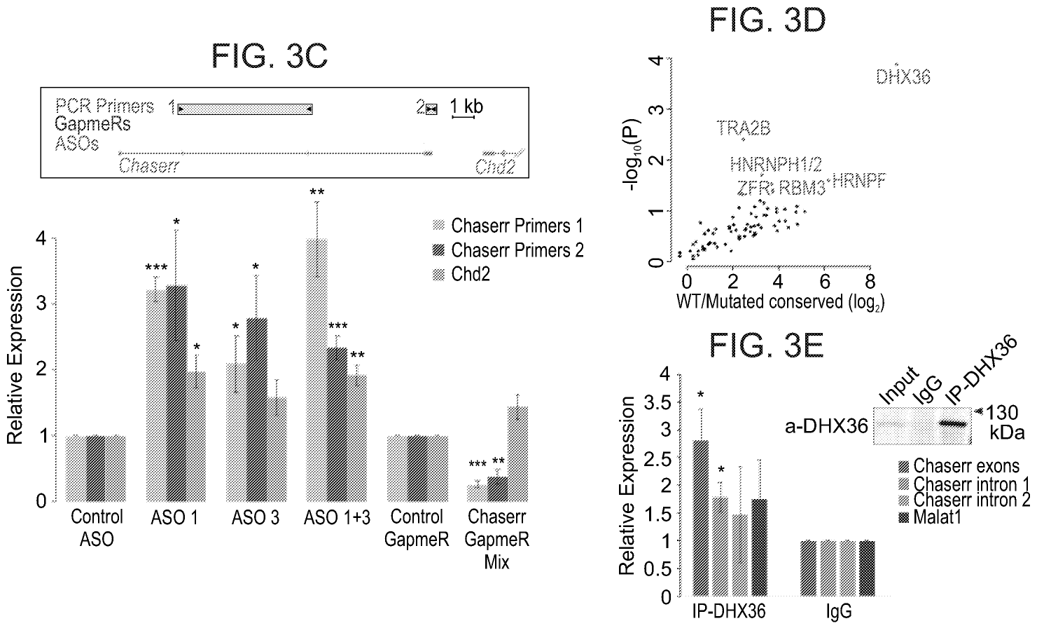

A rich HTML-based suite is used to visualize these motifs in different ways, e.g., color coding them based on depth of conservation, and highlighting motifs in both the query sequence and in the other sequences (see FIGS. 3A-E and 4 for examples of LncLOOM output). The LncLOOM output also includes a color-coded custom track of motifs identified in the query sequence, which can be viewed in the UCSC genome browser. The motifs are annotated using a set of seed sites of conserved microRNAs (from TargetScan) and RBP binding sites found in eCLIP data from the ENCODE project.

### Example 2

LncLOOM Identifies Deeply Conserved Elements in the Cyrano lncRNA

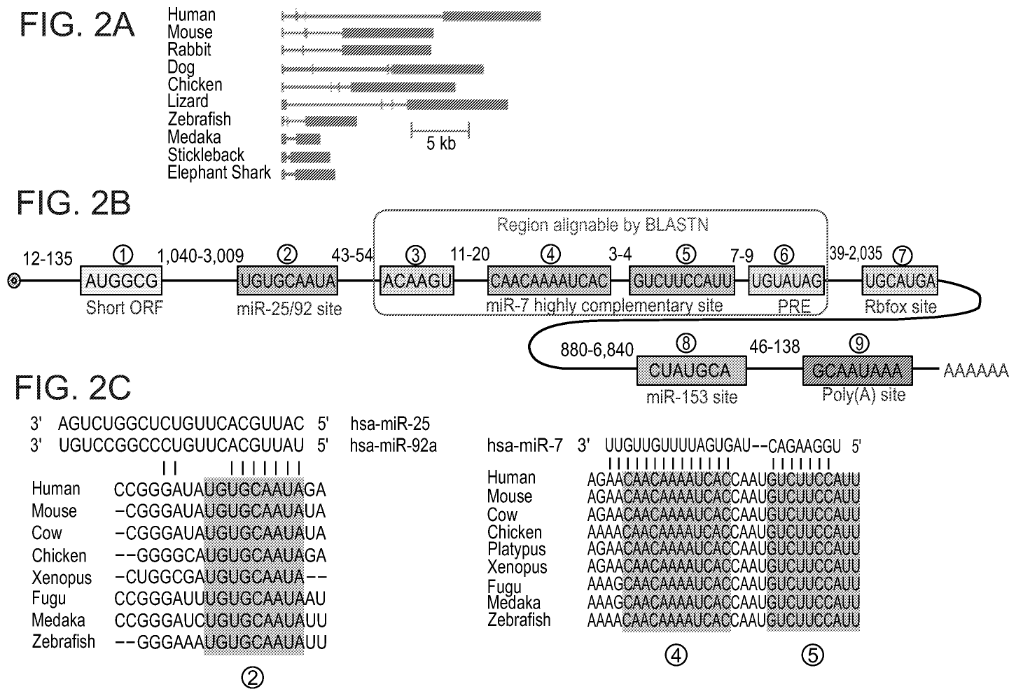

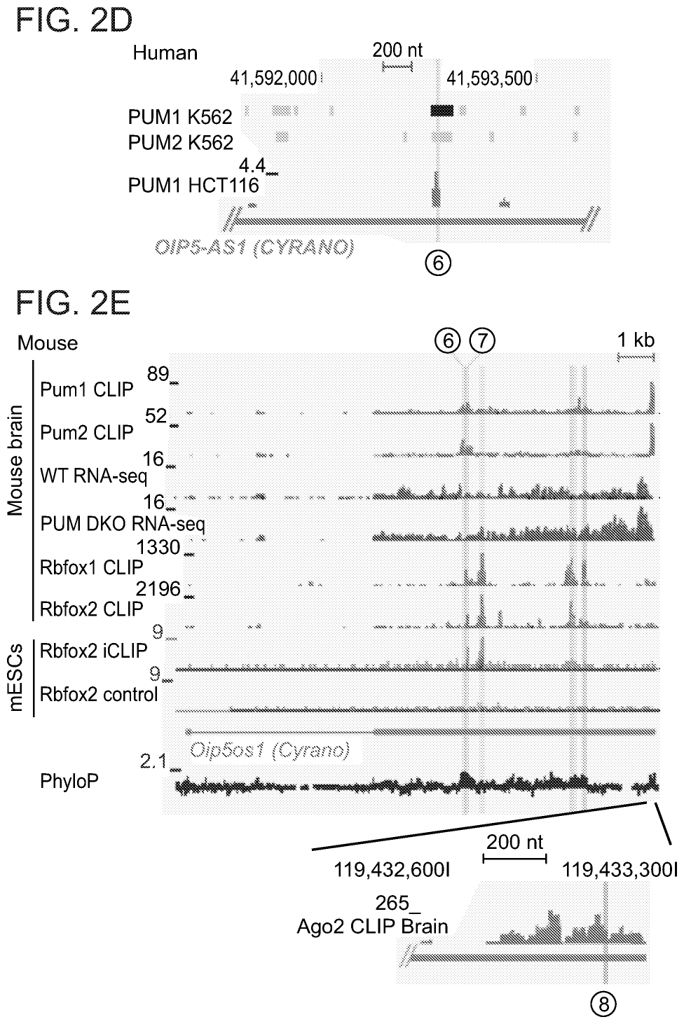

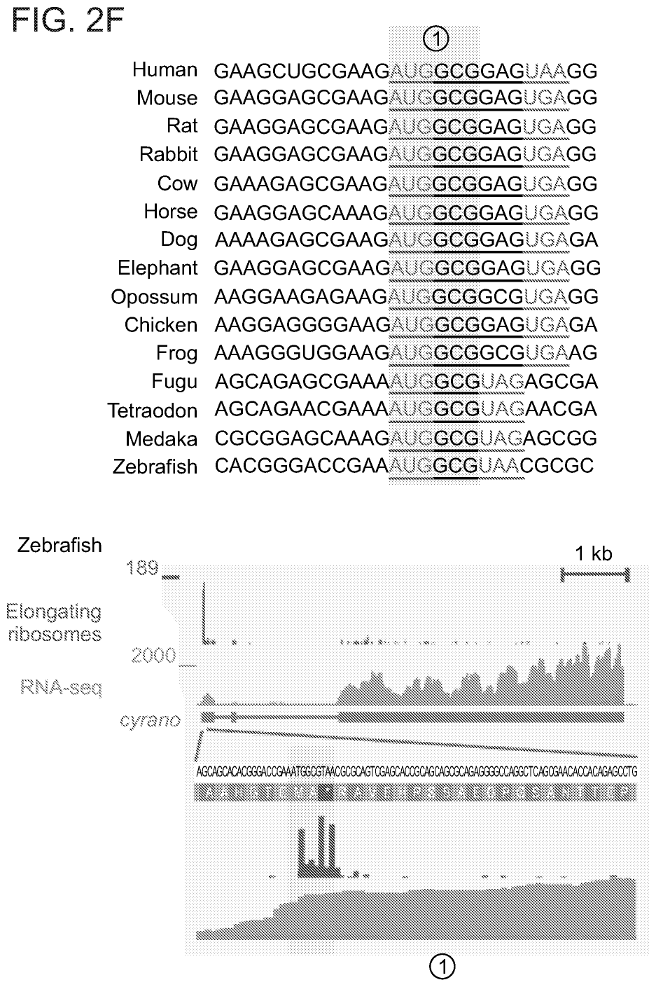

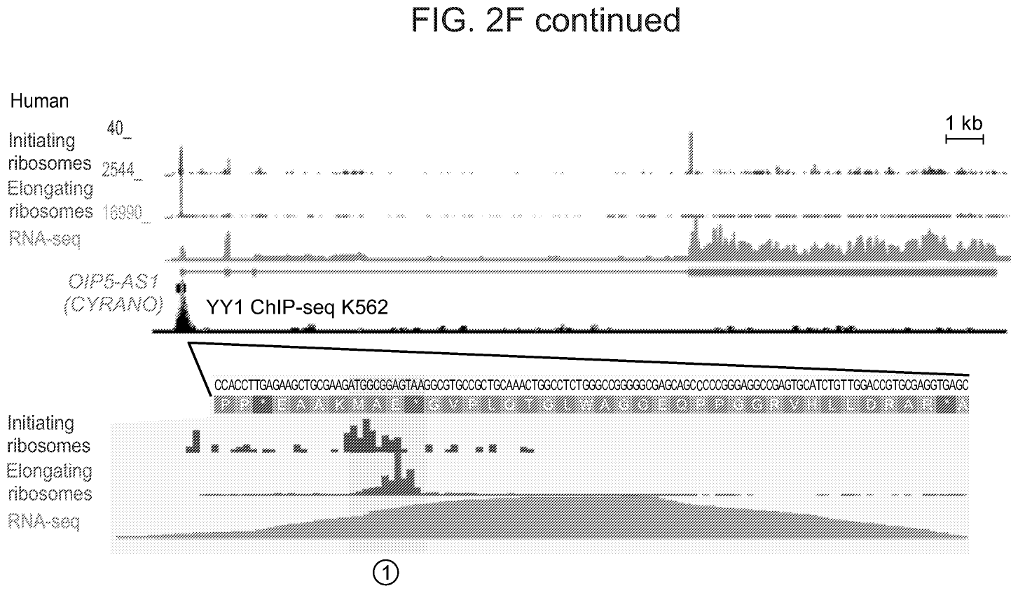

The Cyrano lncRNA is a broadly and highly expressed incRNA^{12,13} Despite being conserved throughout vertebrates, Cyrano exhibits ˜5-fold variation in overall exonic sequence length (2,340 nt in medaka to 10,155 nt in opossum, FIG. 2A). The previously identified 67 nt highly constrained element in Cyrano is the only region that BLAST reports with significant similarity when zebrafish and human sequences are compared. Furthermore, the entire Cyrano locus is not alignable between mammals and fish in the 100-way whole genome alignment (UCSC genome browser). The highly conserved element contains an unusually extensively complementary miR-7 binding site, which is required for degradation of miR-7 by Cyrano.

In order to identify additional conserved elements, Cyrano sequences were curated from 18 species where usable RNA-seq data could be located, including eight mammals, chicken, X. tropicalis, seven vertebrate fish species, and the elephant shark (not shown). LncLOOM identified seven elements conserved in all species, nine conserved in all species except shark (FIG. 2B), and 37 motifs conserved throughout mammals. The following work focuses on the nine elements conserved in all species except shark (numbered 1-9 in FIG. 2B.

(SEQ ID NO: 17)
AUGGCG
(SEQ ID NO: 18)
UGUGCAAUA
(SEQ ID NO: 19)
ACAAGU
(SEQ ID NO: 20)
CAACAAAAU;
(SEQ ID NO: 21)
GUCUUCCAUU;
(SEQ ID NO: 22)
UGUAUAG
(SEQ ID NO: 23)
UGCAUGA
(SEQ ID NO: 24)
CUAUGCA
(SEQ ID NO: 25)
GCAAUAAA,

seven of which were found to be statistically significant by both LncLOOM tests (P<0.01) (as described in materials and methods). Only elements 3-6 fall within the 67 nt conserved region identifiable by BLAST, including two that correspond to pairing with the 5′ and 3′ of miR-7 (FIG. 2C), and another, UGUAUAG (SEQ ID NO: 22), that resembles a Pumilio Recognition Element (PRE, element #6). This element indeed binds PUM1 and PUM2 in CLIP data from human and mouse (FIGS. 2D-E), and in the mouse neonatal brain, where Cyrano levels are relatively high, depletion of Pum1 and Pum2 leads to an increase in Cyrano expression (adjusted P-value 3.49×10⁻³, data from¹⁴, FIG. 2E), consistently with the functions of these proteins in RNA decay¹⁵. This repression is likely due to the combined effect of this highly conserved PRE and others—the 18 Cyrano sequences from different species had 3.2 consensus PREs on average (including two in the mouse sequence, compared to 1.3 on average in 1,000 random shuffled sequences, P<0.001, see Methods).

A putative biological function can be assigned to several additional conserved elements identified by LncLOOM within the Cyrano sequence. A 9mer conserved in all 18 input species, UGUGCAAUA (element #2, SEQ ID NO: 35, in FIG. 2B), is found ˜60 nt upstream of the miR-7 binding site, outside of the region alignable by BLAST. This element corresponds to a miR-25/92 family seed match (FIG. 2C), and was recently shown to be bound and regulated by members of the miR-25/92 family in mouse embryonic heart¹⁶. At the 3′ end of Cyrano, one conserved element (SEQ ID NO: 25, GCAAUAAA) corresponds to the Cyrano polyadenylation signal (PAS) as well as a miR-137 site. Another sequence found ˜100 nt upstream of the PAS, CUAUGCA (SEQ ID NO: 24), corresponds to a seed match of miR-153, and this region is bound by Ago2 in the mouse brain (FIG. 2E). Interestingly, Cyrano levels in HeLa cells are reduced by 41% and 11% following transfection of miR-137 and miR-153, respectively ¹⁷. Cyrano is thus under highly conserved regulation by additional microRNAs beyond the reported interactions with miR-7 and miR-25/92.

˜55 nt downstream of the conserved Pumilio binding site, there is a conserved WGCAUGA motif (W=A/U, SEQ ID NO: 27), that matches the consensus binding motif of the Rbfox RBPs. This motif is bound by Rbfox1/2 in mouse, as are additional regions containing instances of WGCAUGA in the 3′ half of Cyrano (FIG. 2E). In fact, analysis of the 18 Cyrano species showed significant enrichment of WGCAUGA (9.8 instances vs. 4.5 expected by chance, P<0.001, see Methods). In contrast to the miRNA and the Pumilio binding sites, inspection of various RNA-seq datasets of Rbfox1/2 loss-of-function identified no effect on Cyrano levels (not shown), suggesting that the extensive and conserved binding by Rbfox1/2 might affect Cyrano's functionality, rather than expression.

Another highly conserved 6mer, AUGGCG (SEQ ID NO: 17), is found at the very 5′ of Cyrano. Inspection of Cyrano sequences and Ribo-seq data from human, mouse, and zebrafish revealed that this 6mer corresponds to the first two codons of a conserved short 2-3 aa ORF (FIG. 2F). A clear ribosome association is found at the 5′ end of Cyrano at this ORF, with very limited numbers of ribosome protected fragments observed downstream to this element in both human and zebrafish (FIG. 2F), suggesting efficient translation and ribosome release at this short ORE. The context of the AUG start codon in the ORF perfectly matches the 12 bases of the TISU motif, a regulatory element influencing both transcription and translation. TISU is located at the 5′ end of transcripts and acts as a YY1 binding site that may dictate transcription initiation site and as a highly efficient and accurate cap-dependent translation initiator element, for translation that operates without scanning^{18,19}. The genomic region of this motif shows strong YY1 binding to the DNA (FIG. 2F). It is suggested that this motif can have a dual function as a YY1 element regulating Cyrano expression, and as the beginning of the short ORF that may contribute to Cyrano function, as suggested for other lncRNAs²⁰. Overall, putative biological functions could be postulated to eight of the nine conserved elements in Cyrano—four as miRNA binding sites, two as RBP binding sites, one as a conserved short ORF, and one as a PAS. These elements are separated by long stretches of non-conserved sequences (FIG. 2B), which underscores the power of combining LncLOOM with annotations and orthogonal data to uncover lncRNA biology.

### Example 3

LncLOOM Identifies Deeply Conserved Elements in the Libra lncRNA

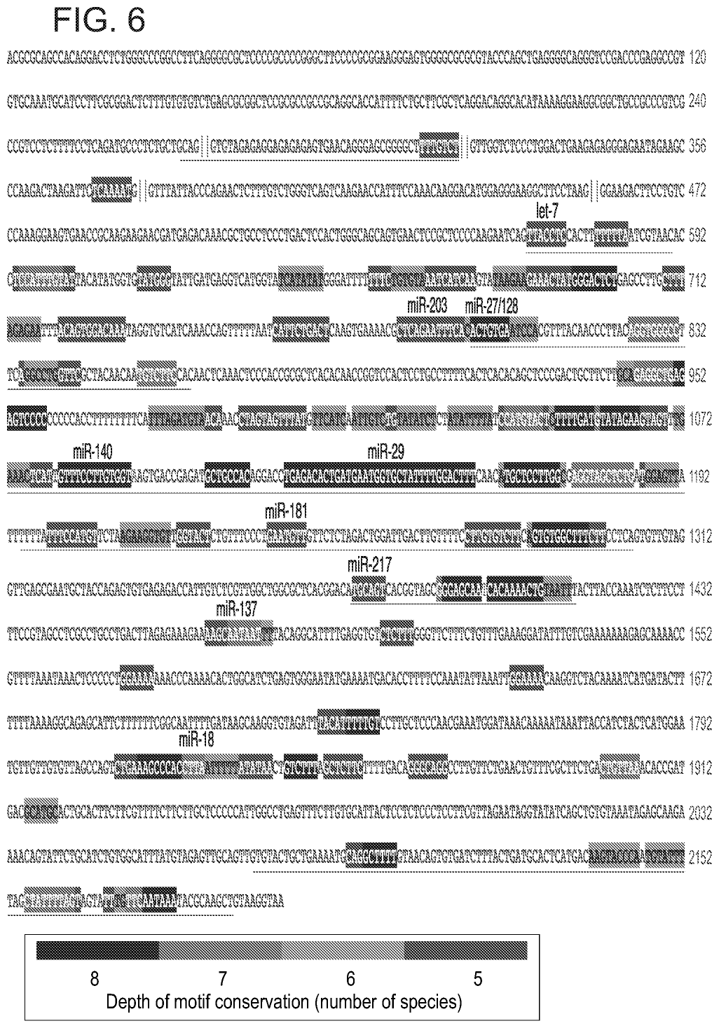

As another example of the ability of LncLOOM to find conserved elements in transcripts known to be associated with the miRNA biology, it was applied on eight homologs of the libra lncRNA in zebrafish and Nrep protein in mammals. This is one of the few examples of a gene that morphed from a likely ancestral lncRNA to a protein-coding gene, while retaining substantial sequence homology in its 3′ region^{12,21}. libra causes degradation of miR-29b in zebrafish and mouse through a highly conserved and highly complementary site²¹. Comparing zebrafish libra with human and mouse sequences using BLASTN recovers an alignment of ˜250 nt from the ˜2.2 kb human sequence, and for spotted gar there are additional short significant alignments (E-value<0.001). LncLOOM found 17 elements conserved between all species, and >25 conserved in all species except zebrafish (FIG. 6). These included the miR-29 site, as well as conserved binding sites for eight additional miRNAs, with three found outside of the region of alignment between mammalian and fish species by BLAST (FIG. 6). It thus appears that Cyrano and libra, the two lncRNAs that were shown to effectively elicit target-directed miRNA degradation (TDMD) harbor several additional highly conserved miRNA binding sites, yet in contrast to the TDMD-mediated sites, these are ‘regular’ seed sites that likely affect lncRNA, rather than miRNA, levels.

### Example 4

LncLOOM Identifies Conserved Motifs in the CHASERR lncRNA

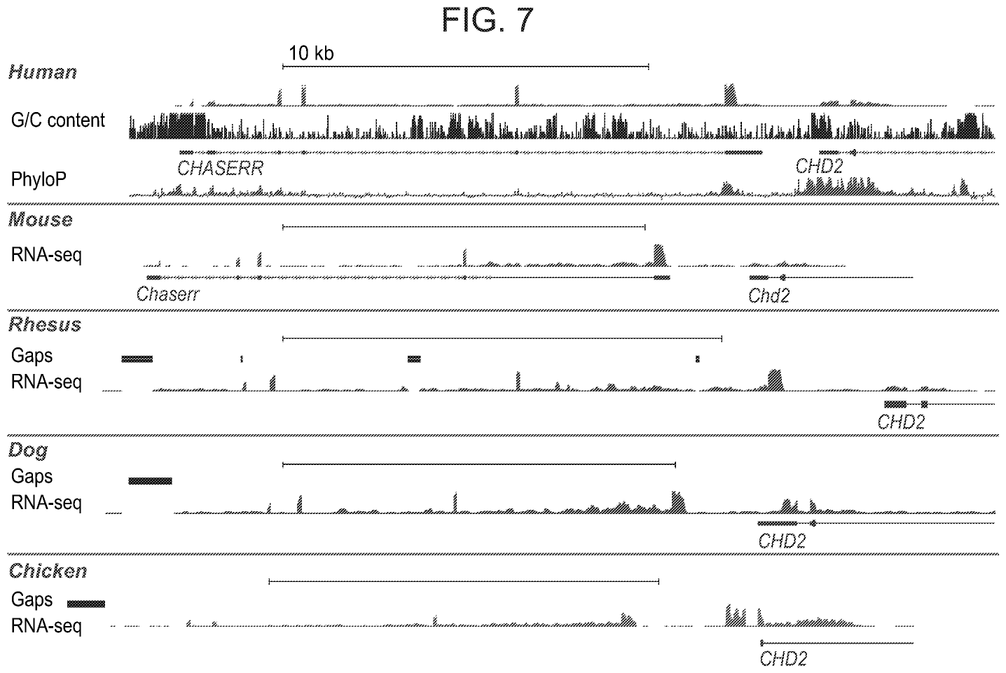

In order to test the ability of LncLOOM to identify conserved modules in sequences that are not amenable for BLAST comparison, the present inventors focused on CHASERR, a lncRNA that was recently characterized as being essential for mouse viability²⁷. CHASERR homologs are readily identifiable in different species based on the close proximity (<2 kb) to the transcription start site of CHD2, as well as their characteristic 5-exon gene architecture²⁷. The present inventors manually curated CHASERR sequences from 16 vertebrates, which were 579-1313 nt in length, and four of which were likely 5′-incomplete due to gaps in some of the genome assemblies around the extremely G/C-rich promoter and first exon of CHASERR²⁷ (FIG. 7). BLASTN found significant (E-value<0.01) alignments between the human CHASERR and the nine sequences coming from amniotes, but not with any of the six other vertebrates. Conversely, when the zebrafish sequence was used as a query, BLAST only found homology in other fish species and in opossum. When the CHASERR sequences are fed into the ClustalO MSA²⁸, only three identical positions are found. The limited conservation of CHASERR is thus a challenge for analysis using commonly-used tools for comparative genomics.

LncLOOM identified two k-mers as conserved in all the layers: AAUAAA (SEQ ID NO: 3) at the 3′ end, which corresponds to the PAS, and AAGAUG (SEQ ID NO: 2), found once or twice in the last exon of all CHASERR sequences (motif 1 in FIG. 3A). The AAUAAA (SEQ ID NO: 1 motif is found near the 3′ end of CHASERR and most likely corresponds to the Polyadenylation Signal (PAS) and was not tested further. Inspection of the CHASERR sequences found that the AAGAUG motif (SEQ ID NO: 5) is substantially overrepresented—CHASERR homologs had 2.1 instances of it on average, compared to merely 0.45 expected by chance (P<0.01). The context of the motif was also typically similar across these 34 instances, with the motif typically followed by a purine (FIG. 3B). An apparently related motif, AUGG (motif 2 in FIG. 3A) (SEQ ID NO: 2), was conserved in 11 of the sequences. Including flanking sequences, motif 2 shares an ARAUGR core with motif 1 (FIG. 3B). It is suggested that these sequences do not match the known binding preference of any RBP, and inspection of eCLIP data did not reveal an obvious candidate for a binder. Therefore the functionality of these sequences was further explored experimentally.

To test the functional significance of the conserved elements, antisense oligonucleotides (ASOs) complementary to the three instances of the conserved motifs in the mouse Chaserr were designed (FIG. 8A), and transfected into mouse Neuro2a (N2a) cells, where it was previously shown that depletion of Chaserr leads to an increase in Chd2 RNA and protein levels²⁷. The human sequences corresponding to these ASOs are CCATAGTAGACTGCCATCTT (SEQ ID NO: 7) targeting AAGATGGCAGTCTACTATGG (SEQ ID NO: 12) and ATCCACTGTCCATTTGTG (SEQ ID NO: 9) targeting CACAAATGGACAGTGGAT (SEQ ID NO: 10).

Transfection of ASO1 and ASO3 individually or mixed led to a significant increase in Chd2 levels, comparable to that caused by knockdown of Chaserr (FIG. 3C). Interestingly, ASO treatment led to an increase in Chaserr levels, as assessed by RT-PCR primer pairs found either upstream or downstream of the ASO-targeted region (FIG. 3C).

In order to identify proteins potentially binding the conserved regions, the present inventors used in vitro transcription to generate biotinylated RNAs containing the WT sequence of the last exon of Chaserr, the same sequence with AUGG-UACC mutations in four conserved motifs, and a second mutant in which all seven of the AUGG sites in the last exon were mutated to UACC (FIG. 8A). These sequences, alongside their antisense controls, were incubated with lysates from N2a cells and proteins that associated with the different RNA variants were isolated and identified using mass spectrometry. As typical in these experiments, a large number of proteins, 938, was identified as associating with the WT sequence (not shown), and 74 of these were enriched ≥3-fold compared to the antisense sequence, however only 9 of these had ≥2-fold higher recovery when using the WT sequence compared to both mutants (FIG. 3D). The present inventors then examined public RNA-seq datasets and sought evidence for changes in Chd2 and/or Chaserr levels when these proteins are perturbed. Such evidence was available for DHX36 and ZFR (FIGS. 8 B-C). The significant association of Chaserr with DHX36—the protein that showed the highest enrichment compared to the mutated sequences—was validated using RNA immunoprecipitation (RIP) and a specific antibody (FIG. 3D). Interestingly, DHX36 is known to bind G-quadruplex sequences^{29,30}, and the conserved elements indeed contain GG pairs, though those are quite far from each other, and typical G-quadruplexes contain runs of at least 3 Gs. QGRS mapper³¹ predicts one G quadruplex in the last exon of Chaserr (FIG. 8A), but other tools including G4RNA scanner³², that integrate different scoring systems did not find any high-scoring G-quadruplexes in the last exon of Chaserr. It is also possible that a non-canonical G quadruplex forming is formed in this sequence, or that it has a different mode of recognition by DHX36.

LncLOOM is therefore capable of identifying functionally relevant elements within lncRNAs that can serve as a basis for design of targeted reagents for perturbing their function, and enabling the use of proteomic methods for identifying specific, functionally relevant, lncRNA interaction partners.

### Example 5

### Deeply Conserved Elements within 3′UTRs of DICER1 and Pumilio mRNAs

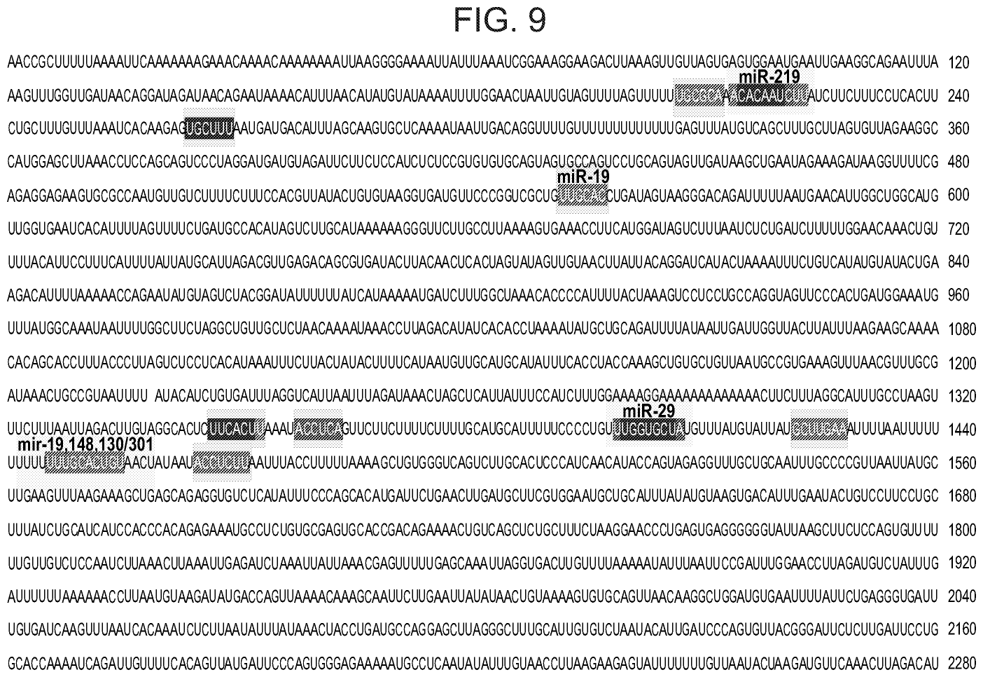

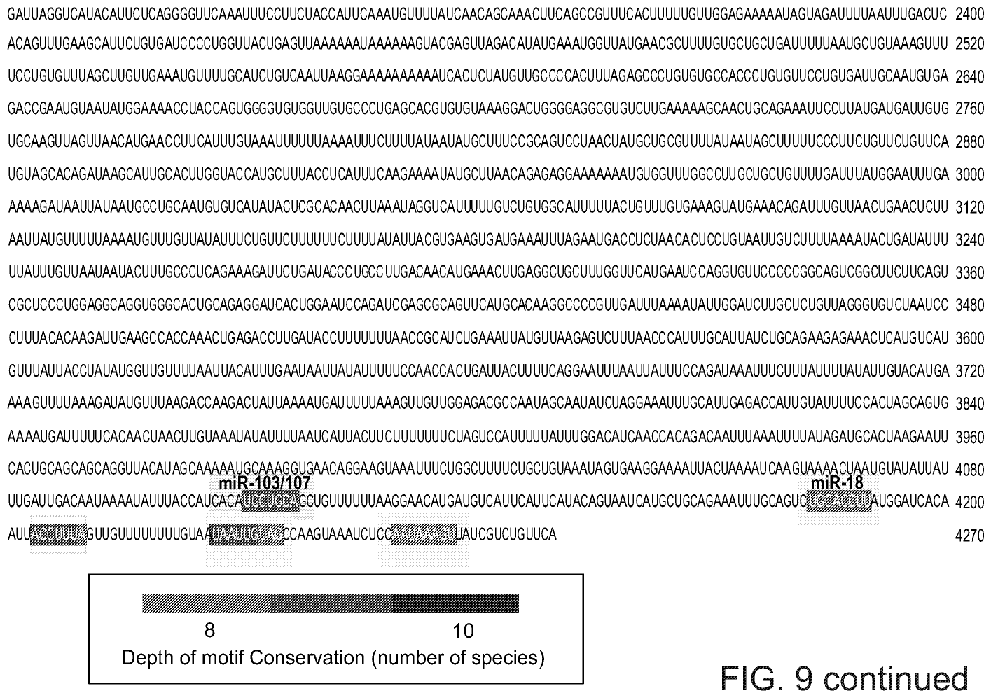

The present inventors next wanted to evaluate the applicability of LncLOOM beyond lncRNAs, and for comparing sequences across longer evolutionary distances. 3′UTRs can dictate RNA stability and translation efficiency of mRNAs, and they typically evolve much more rapidly than other mRNA regions³⁴. Orthology between 3′UTRs is rather easy to define, based on their adjacent coding sequences, which are often readily comparable across very long evolutionary distances. However, there are very few known cases of long-range conservation of functional elements within 3′UTRs between vertebrates and invertebrates. In order to study 3′UTR conservation using LncLOOM, the present inventors first focused on genes that act in post-transcriptional regulation, as these typically undergo particularly complex post-transcriptional regulation. Using available RNA-seq and expressed sequence tag (EST) data, the present inventors compiled a collection of 3′UTR sequences of DICER1, which encodes a key component of the miRNA pathway, from 12 species, including eight vertebrates, lancelet, lamprey, sea urchin, C. intestinalis, and two DICERs in the fruit fly. Human DICER1 could be aligned by BLASTN to the 3′UTRs from vertebrate species, but not beyond. LncLOOM identified 15 elements conserved in all the vertebrate sequences, six with lengths that were not found in random sequences (P<0.01, FIG. 9). Eight of the conserved motifs were conserved beyond vertebrates (and could not be assessed by MSAs or BLAST), and one, corresponding to a binding site for the conserved miR-219 was found in all species, including the fly Dicer2 3′UTR.

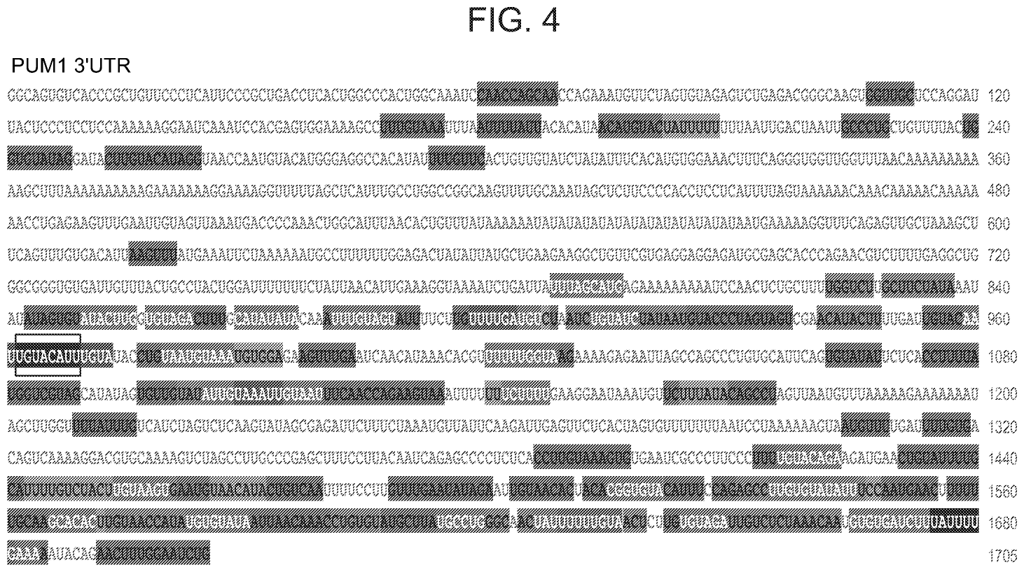

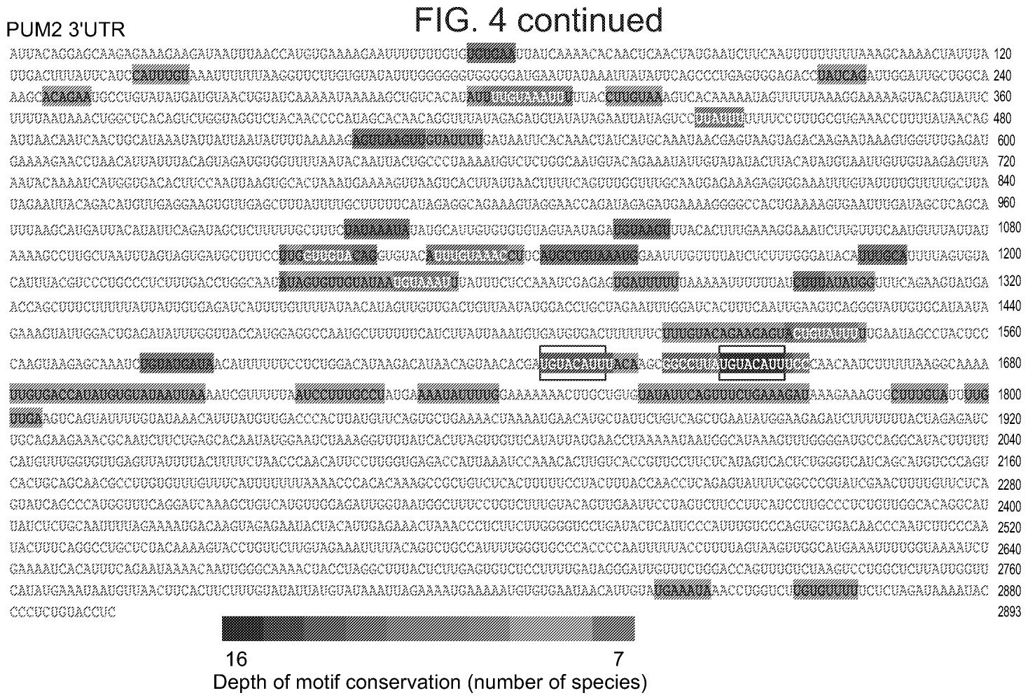

The present inventors then focused on 3′UTRs of the PUM1 and PUM2 mRNAs, which encode Pumilio proteins that post-transcriptionally repress gene expression. Pumilio proteins are deeply conserved, and there are two Pumilio proteins in vertebrates, PUM1 and PUM2, with a single ortholog in other chordates and in flies. 3′UTR sequences from 12 vertebrates and four invertebrates (lamprey, lancelet, C. intestinalis, and fruit fly) were curated. Human and zebrafish 3′UTRs are readily alignable by BLASTN, and there is even significant homology between the 3′UTR of human PUM1 and those of the Pumilio mRNAs in lamprey and lancelet, but not of those in fly and C. intestinalis. LncLOOM identified eight elements conserved throughout vertebrate PUM1 3′UTRs, one of which, UGUACAUU (SEQ ID NO: 14), was conserved in all 16 analyzed 3′UTRs all the way to the fly pum 3′UTR (FIG. 4, top). In PUM2 there were three elements conserved throughout vertebrates, also including UGUACAUU, which was found in all the sequences (FIG. 4, bottom). Interestingly, this UGUACAUU motif partially matches the PRE consensus, UGUANAUA (SEQ ID NO: 28), and it is bound by both PUM1 and PUM2 in human ENCODE data, suggesting that this ancient element is part of the auto-regulatory program that is known to exist in Pumilio mRNAs”. LncLOOM is thus able to identify deeply conserved elements in 3′UTR sequences, including those separated by >500 million years, where available tools do not detect significant sequence conservation.

### Example 6

### Systematic Analysis of Conserved Motifs in 3′UTRs Uncovers Deeply Conserved Elements

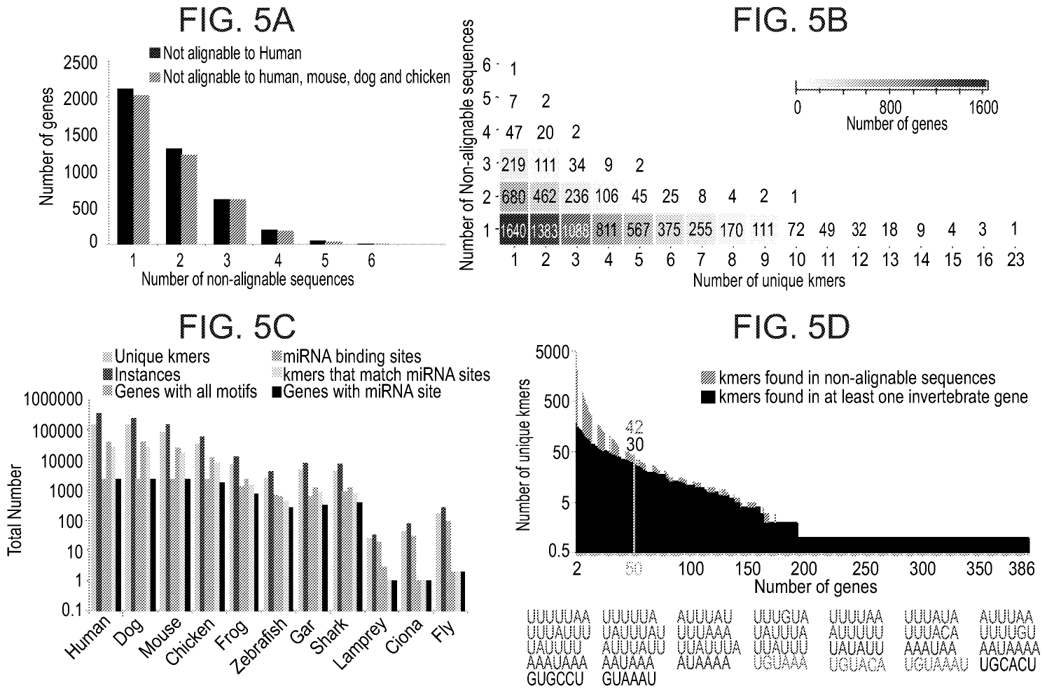

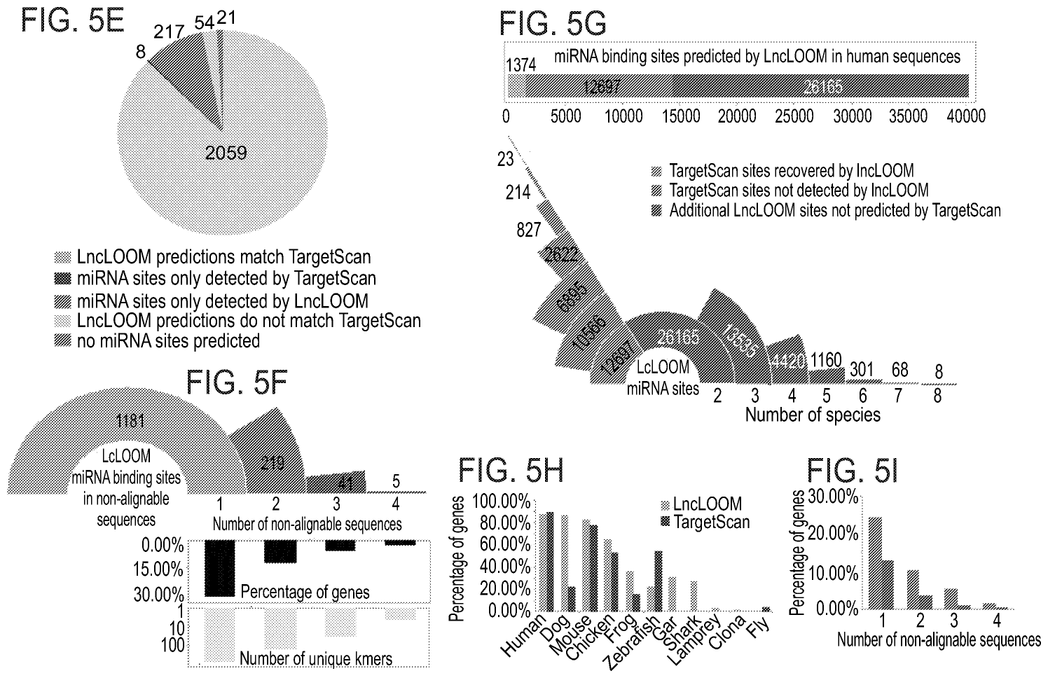

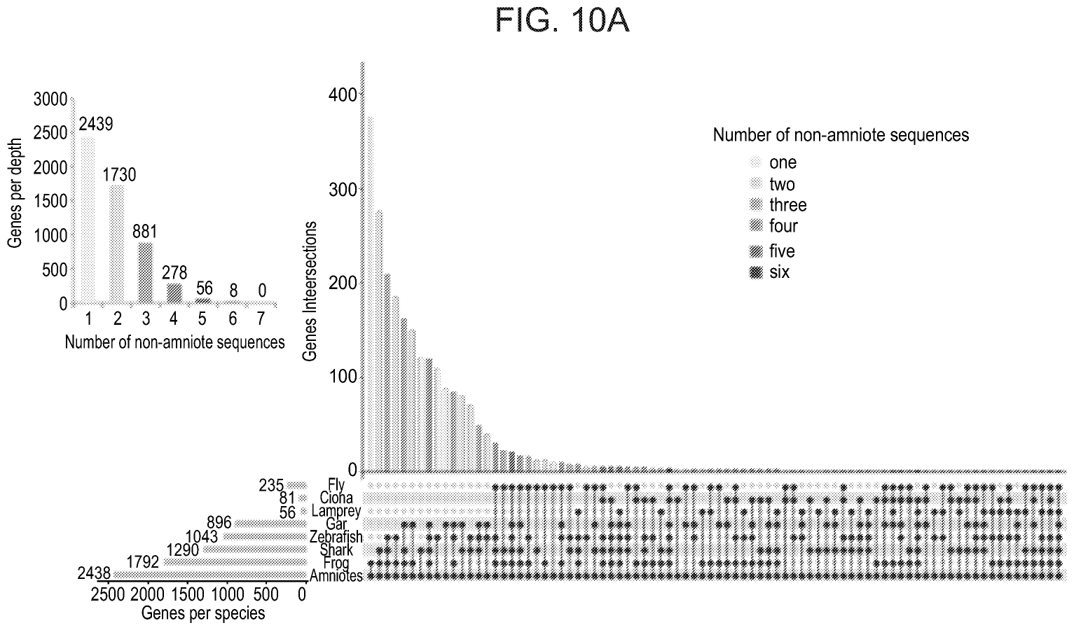

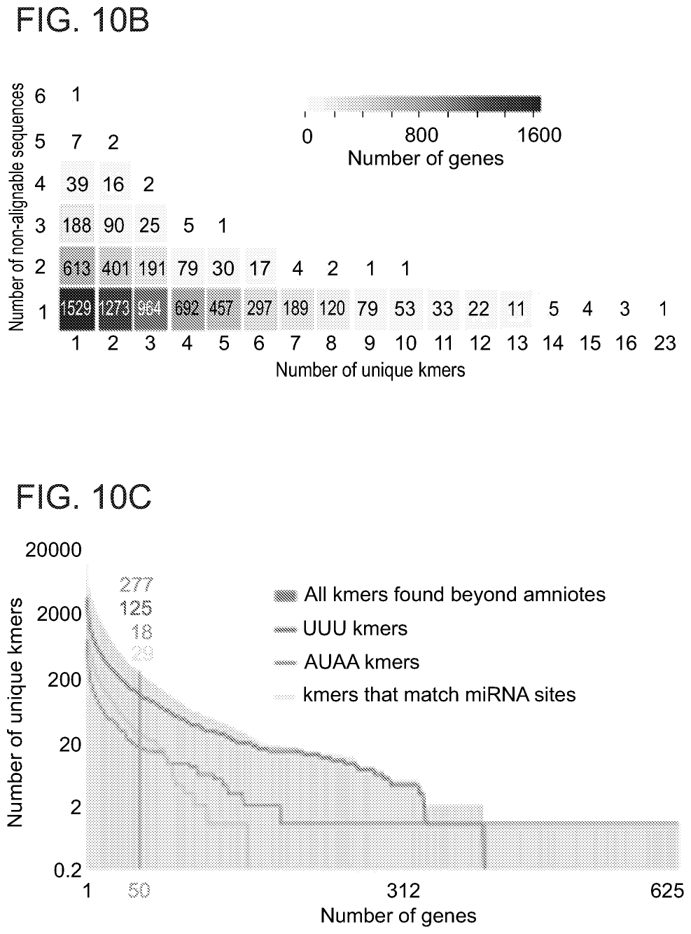

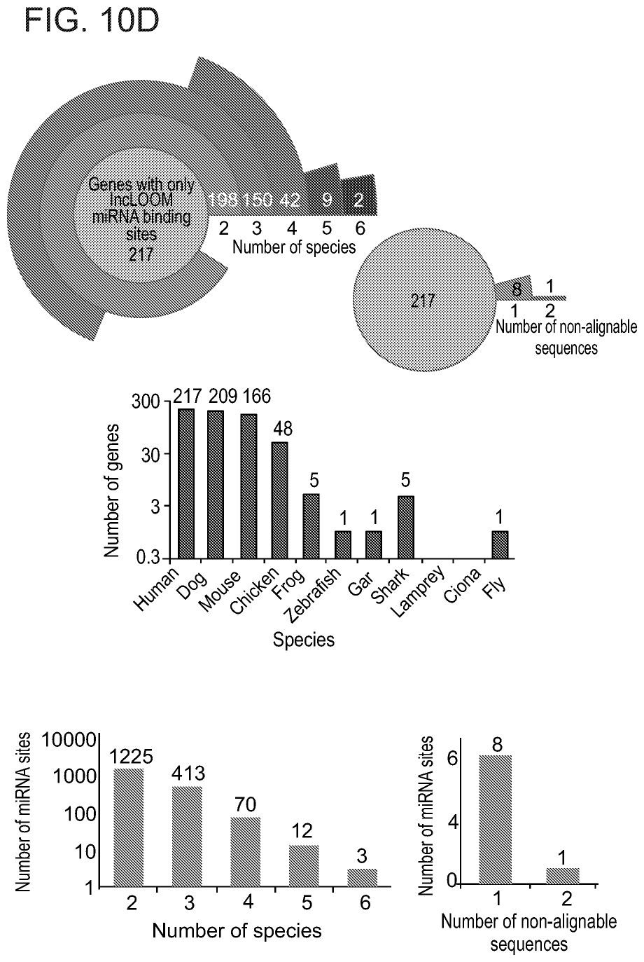

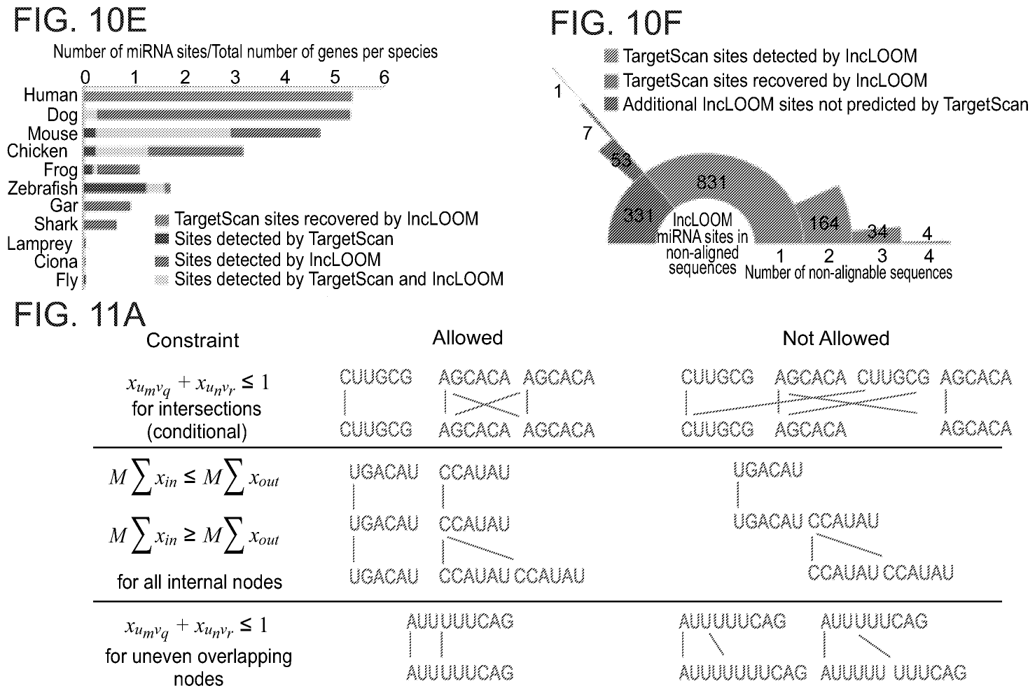

In order to broadly evaluate the predictive power of LncLOOM, a comprehensive analysis of 3′UTR sequences was performed. The present inventors focused on 3′UTRs that are well-defined based on the highly conserved coding sequence flanking them, allowing to build a high-confidence input dataset spanning hundreds of millions of years of evolution, from which it was possible to systematically study thousands of elements using LncLOOM. The dataset was based on 2,439 genes that had 3′UTR MSAs generated as part of the TargetScan7.2 miRNA target site prediction suite ¹⁰. For each gene a dataset of 3′UTR sequences was generated for LncLOOM analysis that contained the aligned sequence from the TargetScan MSA in each of four species (human, mouse, dog, and chicken), only if those were 300-3,000 nt long. For genes with several 3′UTR isoforms the present inventors selected the longest 3′UTR. The present inventors then added to the dataset, where available, sequences of the 3′UTRs annotated in Ensembl in additional species, if those were longer than 200 bases. These included sequences from five non-amniote vertebrate species (frog, shark, zebrafish, gar and lamprey) and two invertebrates (ciona and fly). The main objective was to evaluate the ability of LncLOOM to identify deeply conserved elements, therefore only genes that had a suitable sequence from at least one non-amniote were used. The numbers of sequences that could be analyzed at different depths are presented in FIG. 10A. Of the 2,439 3′UTR datasets, 2,117 contained at least one sequence for which BLASTN did not report any significant alignment (E-value<0.05) to the human sequence, while 2,031 datasets contained at least one sequence that did not have significant alignment to any of the four species (FIG. 5A). Therefore it was possible to analyze a large number of sequences where an MSA-based approach was potentially unable to interrogate the full depth of conservation.

LncLOOM was used to search for conserved motifs with a minimum length of 6 bases and with P<0.05 in all LncLOOM tests. LncLOOM detected over 150,000 significant motifs in the human sequences, of which 27,826 (18.3%) corresponded to a seed site of a broadly conserved miRNA family (as defined by TargetScan). 11,725 k-mers were conserved beyond amniotes, of which 3,897 were detected in at least one non-alignable sequence (FIGS. 5A-I and 10). LncLOOM detected at least one unique k-mer in the first non-alignable layer of 1,640 of the 2,117 genes that contained sequences that did not align to their respective human orthologs, while combinations of at least three unique k-mers were found in 1,088 genes (FIG. 5B). When considering just sequences that did not not align to either of the four amniote species, at least one unique k-mer was detected in the first non-alignable sequence in 1,529 datasets (FIGS. 10A-F). In 114 genes, conservation was found beyond vertebrates and in 97 conservation all the way from human to the fruit fly. A total of 170 unique k-mers (265 instances) were found in fly genes, of which only two matched a broadly conserved miRNA binding site (FIG. 5C).

The present inventors next considered specific conserved k-mers shared between 3′UTRs of multiple genes. Within the k-mers detected in non-alignable sequences, 42 were common to at least 50 genes of which only two corresponded to a broadly conserved miRNA binding site and 30 were conserved in invertebrate sequences (FIG. 5D). Among these 30, 18 k-mers that contained a UUU sequence in an A/U-rich context, resembling AU-rich elements (AREs) and 5 contained AUAA, resembling PASs. Other k-mers contained an UGUA core, that resembles a PRE. These three groups of miRNA-unrelated elements are thus also often very deeply conserved in 3′UTRs, and these conserved occurrences can be detected by LncLOOM.

To assess the sensitivity of LncLOOM, the binding sites of broadly conserved miRNAs that were identified by LncLOOM were compared to TargetScan predictions for each of the 2,439 genes, in 2,121 of which TargetScan predicted binding sites in the human sequences. lncLOOM predicted binding sites in 2,330 genes, including 217 for which the TargetScan alignments did not identify any broadly conserved sites (FIG. 5E). A summary of all miRNA sites predicted by lncLOOM can be found at github(dot)com/LncLOOM/LncLOOM. In a substantial number of cases (29% of the 2,117 genes), LncLOOM found a miRNA binding site significantly conserved in species where the 3′UTR was not alignable to the human sequence in the MSA (FIG. 5F). To compare lncLOOM and TargetScan predictions more precisely, the present inventors focused on the 2,359 genes for which TargetScan predicted binding sites in the identical human transcript used for lncLOOM analysis (FIG. 5E), amongst which lncLOOM recovered 90.24% of all broadly conserved sites predicted by TargetScan in the human sequences (FIG. 5G). Within the 217 genes, 42 had sites conserved beyond mammals and in several genes conservation was found in fish and fruit fly species (FIGS. 10A-F). In addition to the miRNA sites recovered, lncLOOM identified a further 21,615 broadly conserved sites that had not been previously predicted. When comparing the depth of conservation, incLOOM often detected the sites recovered by TargetScan in more distal species (FIGS. 5G and 10A-F). Importantly, 831 recovered and 331 new predictions were detected in non-alignable sequences in 24% and 13% of genes respectively.

Hence, LncLOOM is a powerful tool also for analysis of 3′UTR sequences, revealing a greater depth of conservation of miRNA or other functional binding sites than what is possible by MSA-based approach while having only a limited compromise on sensitivity.

### Example 7

### Targeting of CHASERR Causes Upregulation of CHD2 in Neuroblastic Cells

Sequences are provided infra:

Human Chaserr
(SEQ ID NO: 123)
AAGGGGUAUCAUCUGACGGUAGAACUAA 5′
Mouse Chaserr
(SEQ ID NO: 124)
AAGGGGUAUUACCCGACGGUAGAACUAA 5′
A40/A52
(SEQ ID NO: 128/133)
5′ CCAUAGUAGACUGCCAUCUU 3′
A50
(SEQ ID NO: 131)
5′ CCAUAGUAGACUGCCAUC 3′
A51
(SEQ ID NO: 132)
5′ AUAGUAGACUGCCAUCUU 3′
A35
(SEQ ID NO: 127)
5′ CCAUAAUGGGCUGCCAUCUU 3′
A49
(SEQ ID NO: 130)
5′ CCAUAGUGGGCUGCCAUCUU 3′
A27
(SEQ ID NO: 125)
5′ CGAUAGCAGGAGAAGUCUGAAG 3′
A28
(SEQ ID NO: 126)
5′ CUCUCUCUCUUUCUAUCCCUUC 3′

### ASOs Targeting CHASERR:

A35—the same ASO as the one used in mouse. This ASO is complementary to the mouse sequence.
A40—an ASO targeting the same region as ASO1 in mouse, but fully complementary to the human sequence.
A49—an ASO similar to the A35 and A40, but which has the potential to base pair with both the human and the mouse sequence using G-U pairing.
A50—identical to A40, but with 2′MO modifications instead of 2′MOE and truncated by 2 bases at 3′end
A51—identical to A40, but with 2′MO modifications instead of 2′MOE and truncated by 2 bases at 5′end
A52—identical to A40, but including LNA modifications

### Results

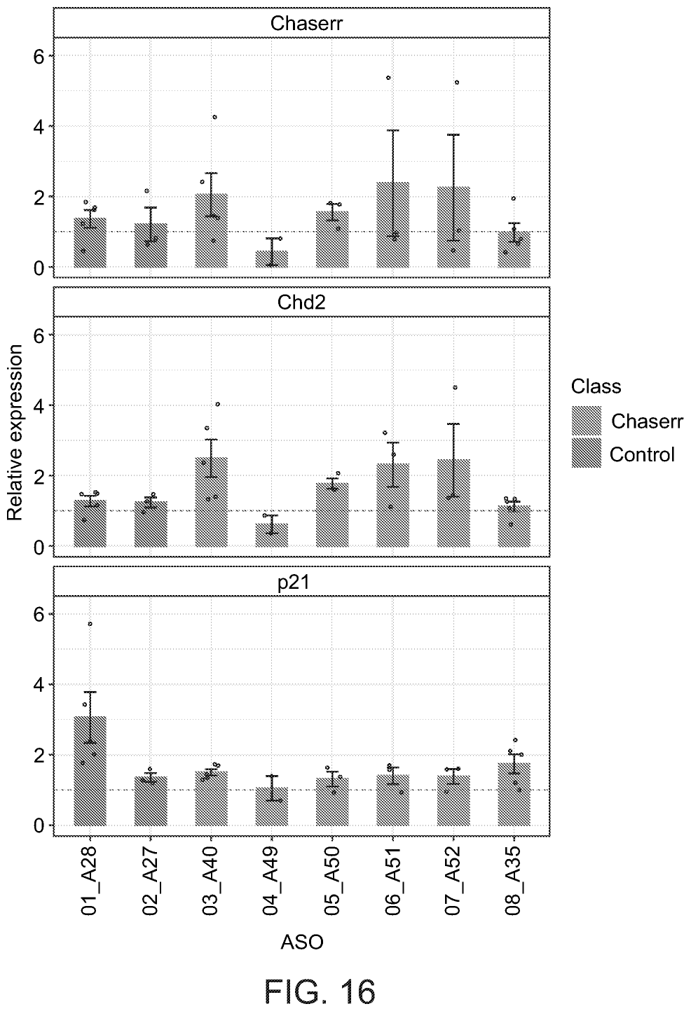

The effects on CHD2 mRNA and protein levels were compared to a non-targeting ASOs A27 and A28. A28 is causing up-regulation of p21 and stress response in SH-SY5Y cells (FIG. 16), therefore the comparison was done to A27.

Cells were plated at a density of 2.5×10⁵/35 mm plate. The cells were transfected with 25 nM of ASO using DharmaFECT4 transfection reagent (T-2004-03, horizon). RNA was extracted 48 hrs post-transfection.

ASOs A40, A50, A51, and A52 were most potent in up-regulating CHD2 relative to untransfected cells or cells transfected with the control ASOs (FIG. 16).

### Example 8

### Targeting of CHASERR Causes Upregulation of CHD2 in MCF7 Cells and SH-SY5Y

### Antisense Oligonucleotide and LNA GapmeR Transfections

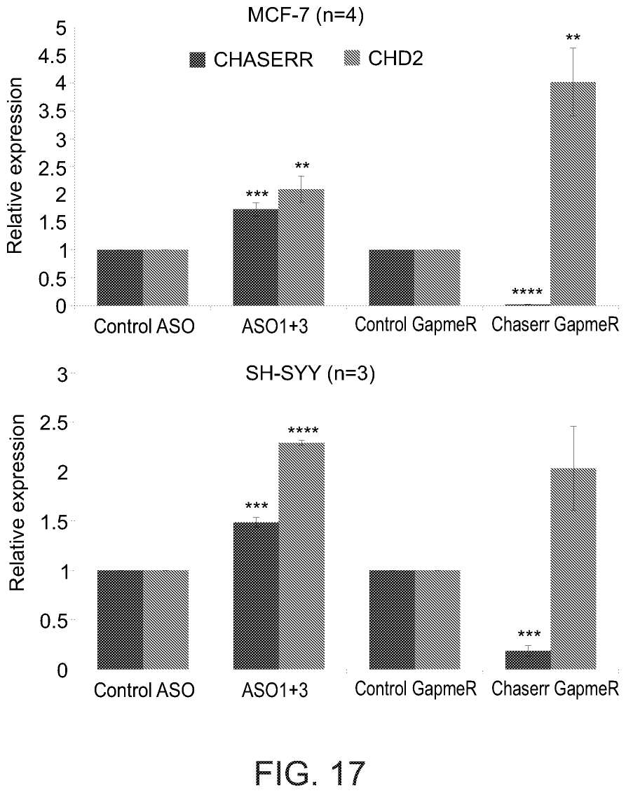

MCF7 cell lines (obtained from the ATCC) were cultured in DMEM containing 10% fetal bovine serum and 100 U penicillin/0.1 mg ml⁻¹ streptomycin. SH-SY5Y cell lines (obtained from the ATCC) were cultured in DMEM/Nutrient Mixture F-12 Ham (Sigma: D6421) containing 10% fetal bovine serum, 100 U penicillin/0.1 mg ml⁻¹ streptomycin and 2 mM GlutaMAX (Thermofisher: 35050061). All cells were cultured at 37° C. in a humidified incubator with 5% CO₂ and routinely tested for mycoplasma contamination. The first set of ASOs: ASO1 (A40, SEQ ID NO: 128) and ASO3 (A41, SEQ ID NO: 134) were modified with 2′-O-methoxy-ethyl bases. An LNA gapmer, targeted to the second intron of human Chaserr was used for Chaserr knockdown. Transfection: 2×10⁵ MCF7 or SH-SY5Y were seeded in a six-well plate and transfected using Dharmafect4 (Dharmacon) transfection reagent following the manufacturer's protocol with either a mix of ASO1 (ASO40) and ASO3 (ASO41) or with the Chaserr gapmeR (Table 5) to a final concentration of 50 nM. Endpoints for all experiments were at 48 h post transfection, after which the cells were collected with TRIZOL for RNA extraction and assessment by RT-qPCR analysis. The effect on Chasser and CHD2 expression is shown in FIG. 17.

TABLE 5
Oligonucleotide sequences of ASOs and LNA GapmeRs

| Name | Sequence/SEQ ID NO: |
|---|---|
| ASO1 (ASO40) | CCAUAGUAGACUGCCAUCUU/128 |
| ASO3 (ASO41) | ATCCACUGUCCAUUUGTG/134 |
| Control ASO (A28) | CGAUAGCAGGAGAAGUCUGAAG/126 |
| Chaserr GapmeR | GTCGAATAAACCAGTATC/135 |
| Control GapmeR | AACACGTCTATACGC |
| | (Cat#: LG00000002)/136 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other References are Included in the Text)

1. Ulitsky, I. & Bartel, D. P. lincRNAs: genomics, evolution, and mechanisms. Cell 154, 26-46 (2013).
2. Iyer, M. K. et al. The landscape of long noncoding RNAs in the human transcriptome. Nat. Genet. 47, 199-208 (2015).
3. Ulitsky, I. Evolution to the rescue: using comparative genomics to understand long non-coding RNAs. Nat. Rev. Genet. (2016) doi:10.1038/nrg.2016.85.
4. Hezroni, H. et al. Principles of Long Noncoding RNA Evolution Derived from Direct Comparison of Transcriptomes in 17 Species. Cell Rep. (2015) doi:10.1016/j.celrep.2015.04.023.
5. Wang, A. X., Ruzzo, W. L. & Tompa, M. How accurately is ncRNA aligned within whole-genome multiple alignments? BMC Bioinformatics 8, 417 (2007).
6. Bartel, D. P. Metazoan MicroRNAs. Cell 173, 20-51 (2018).
7. Dominguez, D. et al. Sequence, Structure, and Context Preferences of Human RNA Binding Proteins. Mol. Cell 70, 854-867.e9 (2018).
8. Maier, D. The Complexity of Some Problems on Subsequences and Supersequences. (1978).
9. AtamtQrk, A. & Savelsbergh, M. W. P. Integer-Programming Software Systems. Ann. Oper. Res. 140, 67-124 (2005).
10. Agarwal, V., Bell, G. W., Nam, J.-W. & Bartel, D. P. Predicting effective microRNA target sites in mammalian mRNAs. Elife 4, e05005 (2015).
11. Van Nostrand, E. L. et al. A Large-Scale Binding and Functional Map of Human RNA Binding Proteins. bioRxiv 179648 (2017) doi:10.1101/179648.
12. Ulitsky, I., Shkumatava, A., Jan, C. H., Sive, H. & Bartel, D. P. Conserved function of lincRNAs in vertebrate embryonic development despite rapid sequence evolution. Cell 147, 1537-1550 (2011).
13. Kleaveland, B., Shi, C. Y., Stefano, J. & Bartel, D. P. A Network of Noncoding Regulatory RNAs Acts in the Mammalian Brain. bioRxiv (2018).
14. Zhang, M. et al. Post-transcriptional regulation of mouse neurogenesis by Pumilio proteins. Genes Dev. 31, 1354-1369 (2017).
15. Goldstrohm, A. C., Hall, T. M. T. & McKenney, K. M. Post-transcriptional Regulatory Functions of Mammalian Pumilio Proteins. Trends Genet. 34, 972-990 (2018).
16. Li, X., Pritykin, Y., Concepcion, C. P., Lu, Y. & La Rocca, G. High-resolution in vivo identification of miRNA targets by Halo-Enhanced Ago2 Pulldown. bioRxiv (2019).
17. McGeary, S. E., Lin, K. S., Shi, C. Y., Bisaria, N. & Bartel, D. P. The biochemical basis of microRNA targeting efficacy. doi:10.1101/414763.
18. Elfakess, R. & Dikstein, R. A translation initiation element specific to mRNAs with very short 5′UTR that also regulates transcription. PLoS One 3, e3094 (2008).
19. Elfakess, R. et al. Unique translation initiation of mRNAs-containing TISU element. Nucleic Acids Res. 39, 7598-7609 (2011).
20. Housman, G. & Ulitsky, I. Methods for distinguishing between protein-coding and long noncoding RNAs and the elusive biological purpose of translation of long noncoding RNAs. Biochim. Biophys. Acta (2015) doi:10.1016/j.bbagrm.2015.07.017.
21. Bitetti, A. et al. MicroRNA degradation by a conserved target RNA regulates animal behavior. Nat. Struct. Mol. Biol. 25, 244-251 (2018).
22. Munschauer, M. et al. The NORAD lncRNA assembles a topoisomerase complex critical for genome stability. Nature 561, 132-136 (2018).
23. Lovci, M. T. et al. Rbfox proteins regulate alternative mRNA splicing through evolutionarily conserved RNA bridges. Nat. Struct. MoL. Biol. 20, 1434-1442 (2013).
24. Jangi, M., Boutz, P. L., Paul, P. & Sharp, P. A. Rbfox2 controls autoregulation in RNA-binding protein networks. Genes Dev. 28, 637-651 (2014).
25. Chi, S. W., Zang, J. B., Mele, A. & Darnell, R. B. Argonaute HITS-CLIP decodes microRNA-mRNA interaction maps. Nature 460, 479-486 (2009).
26. Michel, A. M. et al. GWIPS-viz: development of a ribo-seq genome browser. Nucleic Acids Res. 42, D859-64 (2014).
27. Rom, A. et al. Regulation of CHD2 expression by the Chaserr long noncoding RNA gene is essential for viability. Nat. Commun. 10, 5092 (2019).
28. Sievers, F. et al. Fast, scalable generation of high-quality protein multiple sequence alignments using Clustal Omega. Mol. Syst. Biol. 7, (2011).
29. Chen, M. C. et al. Structural basis of G-quadruplex unfolding by the DEAH/RHA helicase DHX36. Nature 558, 465-469 (2018).
30. Sauer, M. et al. DHX36 prevents the accumulation of translationally inactive mRNAs with G4-structures in untranslated regions. Nat. Commun. 10, 2421 (2019).
31. Kikin, O., D'Antonio, L. & Bagga, P. S. QGRS Mapper: a web-based server for predicting G-quadruplexes in nucleotide sequences. Nucleic Acids Res. 34, W676-82 (2006).
32. Garant, J.-M., Perreault, J.-P. & Scott, M. S. G4RNA screener web server: User focused interface for RNA G-quadruplex prediction. Biochimie vol. 151 115-118 (2018).
33. Haque, N., Ouda, R., Chen, C., Ozato, K. & Hogg, J. R. ZFR coordinates crosstalk between RNA decay and transcription in innate immunity. Nat. Commun. 9, 1145 (2018).
34. Shabalina, S. A., Ogurtsov, A. Y., Rogozin, I. B., Koonin, E. V. & Lipman, D. J. Comparative analysis of orthologous eukaryotic mRNAs: potential hidden functional signals. Nucleic Acids Res. 32, 1774-1782 (2004).
35. Kirk, J. M. et al. Functional classification of long non-coding RNAs by k-mer content. Nat. Genet. 50, 1474-1482 (2018).
36. Quinn, J. J. et al. Rapid evolutionary turnover underlies conserved lncRNA-genome interactions. Genes Dev. 30, 191-207 (2016).
37. Tycowski, K. T., Shu, M. D., Borah, S., Shi, M. & Steitz, J. A. Conservation of a triple-helix-forming RNA stability element in noncoding and genomic RNAs of diverse viruses. Cell Rep. 2, 26-32 (2012).
38. Deveson, I. W. et al. Universal Alternative Splicing of Noncoding Exons. Cell Syst 6, 245-255.e5 (2018).
39. Katoh, K., Misawa, K., Kuma, K.-I. & Miyata, T. MAFFT: a novel method for rapid multiple sequence alignment based on fast Fourier transform. Nucleic Acids Res. 30, 3059-3066 (2002).
40. Altschul, S. F., Gish, W., Miller, W., Myers, E. W. & Lipman, D. J. Basic local alignment search tool. J. MoL. Biol. 215, 403-410 (1990).
41. Karp, R. M. Reducibility among Combinatorial Problems. in Complexity of Computer Computations: Proceedings of a symposium on the Complexity of Computer Computations, held Mar. 20-22, 1972, at the IBM Thomas J. Watson Research Center, Yorktown Heights, New York, and sponsored by the Office of Naval Research, Mathematics Program, IBM World Trade Corporation, and the IBM Research Mathematical Sciences Department (eds. Miller, R. E., Thatcher, J. W. & Bohlinger, J. D.) 85-103 (Springer US, 1972).
42. Hagberg, A., Swart, P. & S Chult, D. Exploring network structure, dynamics, and function using NetworkX. www(dot)osti(dot)gov/biblio/960616 (2008).
43. Mitchell, S., OSullivan, M. & Dunning, I. PuLP: a linear programming toolkit for python. The University of Auckland, Auckland, New Zealand (2011).
44. Kent, W. J. BLAT-The BLAST-Like Alignment Tool. Genome Res. 12, 656-664 (2002).
45. Dobin, A. et al. STAR: ultrafast universal RNA-seq aligner. Bioinformatics 29, 15-21 (2013).
46. Li, B. & Dewey, C. N. RSEM: accurate transcript quantification from RNA-Seq data with or without a reference genome. BMC Bioinformatics 12, 323 (2011).
47. Elinger, D., Gabashvili, A. & Levin, Y. Suspension Trapping (S-Trap) Is Compatible with Typical Protein Extraction Buffers and Detergents for Bottom-Up Proteomics. J. Proteome Res. 18, 1441-1445 (2019).
48. Cox, J. & Mann, M. MaxQuant enables high peptide identification rates, individualized p.p.b.-range mass accuracies and proteome-wide protein quantification. Nat. Biotechnol. 26, 1367-1372 (2008).

What is claimed is:

1. A method of increasing an amount of Chromodomain Helicase DNA Binding Protein 2 (CHD2) in a neuronal cell, the method comprising introducing into the cell a nucleic acid agent that down-regulates activity or expression of human Chaserr, wherein the nucleic acid agent is directed at the last exon of human Chaserr, thereby increasing the amount of CHD2 in the neuronal cell.

2. A method of treating a disease or medical condition associated with Chromodomain Helicase DNA Binding Protein 2 (CHD2) haploinsufficiency in a subject in need thereof, the method comprising administering to the subject a therapeutically effective amount of a nucleic acid agent that down-regulates activity or expression of human Chaserr, wherein the nucleic acid agent is directed at the last exon of human Chaserr, thereby treating the disease or medical condition associated with CHD2 haploinsufficiency.

3. A nucleic acid agent that down-regulates activity or expression of human Chaserr for use in treating a disease or medical condition associated with Chromodomain Helicase DNA Binding Protein 2 (CHD2) haploinsufficiency in a subject in need thereof, wherein the nucleic acid agent is directed at the last exon of human Chaserr.

4. A nucleic acid agent that activity or expression of human Chaserr, wherein the nucleic acid agent comprises a nucleic acid sequence that hybridizes at the last exon of human Chaserr.

5. The method or nucleic acid agent for use, or nucleic acid agent of any one of claims 1-4, wherein said human Chaserr comprises an alternatively spliced variant selected from the group consisting of SEQ ID NO: 11 (NR_037600), SEQ ID NO: 12 (NR_037601), and SEQ ID NO: 13 (NR_037602).

6. The method or nucleic acid agent for use, or nucleic acid agent of any one of claims 1-5, wherein said nucleic acid agent comprises a sequence that is complementary to SEQ ID NO: 2 (AUGG).

7. The method or nucleic acid agent for use, or nucleic acid agent of any one of claims 1-5, wherein said nucleic acid agent comprises a sequence that is complementary to AAGAUG (SEQ ID NO: 5) or AAAUGGA (SEQ ID NO: 6).

8. The method or nucleic acid agent for use, or nucleic acid agent of any one of claims 1-5, wherein said nucleic acid agent comprises a sequence that is complementary to UUUUUACCU (SEQ ID NO: 122).

9. The method or nucleic acid agent for use, or nucleic acid agent of any one of claims 1-8, wherein said nucleic acid agent inhibits binding of DHX36 to Chaserr.

10. The method or nucleic acid agent for use, or nucleic acid agent of any one of claims 1-8, wherein said nucleic acid agent inhibits binding of CHD2 to Chaserr.

11. The method or nucleic acid agent for use, or nucleic acid agent of any one of claims 1-9, wherein said nucleic acid agent is an antisense oligonucleotide.

12. The method or nucleic acid agent for use, or nucleic acid agent of any one of claims 1-11, wherein said nucleic acid agent comprises one or more nucleotides having a 2′ to 4′ bridge, and/or one or more nucleotides having a 2′-0 modification.

13. The method or nucleic acid agent for, or nucleic acid agent use of claim 9, wherein said antisense oligonucleotide is as set forth in SEQ ID NO: 92-99.

14. The method or nucleic acid agent for use, or nucleic acid agent of claim 10 or 12, wherein said antisense oligonucleotide is as set forth in SEQ ID NO: 128, 131, 132, 133, 140, 141, 142 or 143.

15. The method or nucleic acid agent for use, or nucleic acid agent of any one of claims 11, 12 and 13, wherein said antisense oligonucleotide comprises at least 2 antisense oligonucleotides.

16. The method or nucleic acid agent for use, or nucleic acid agent of claim 15, wherein said at least 2 antisense oligonucleotides comprise ASO40 of SEQ ID NO: 140 or 128 and ASO41 of SEQ ID NO: 144 or 134.

17. The method or nucleic acid agent for use, or nucleic acid agent of any one of claims 1-10, wherein said nucleic acid agent is an RNA silencing agent.

18. The method or nucleic acid agent for use, or nucleic acid agent of any one of claims 1-10, wherein said nucleic acid agent is a genome editing agent.

19. The method or nucleic acid agent for use, or nucleic acid agent of any one of claims 1-18, wherein said nucleic acid agent is active in an inducible manner.

20. The method or nucleic acid agent for use, or nucleic acid agent of any one of claims 1-10, wherein said nucleic acid agent is active in a tissue or cell-specific manner.

21. The method or nucleic acid agent for use, or nucleic acid agent of any one of claims 2-20, wherein said disease or medical condition associated with Chromodomain Helicase DNA Binding Protein 2 (CHD2) haploinsufficiency is selected from the group consisting of intellectual disability, autism, epilepsy and Lennox-Gastaut syndrome (LGS).

22. A method of analyzing a set of sequences describing a plurality of homologous polynucleotides, the method comprising:
constructing a graph having a plurality of nodes arranged in layers, and a plurality of edges connecting nodes of consecutive layers, wherein each layer represents a sequence of the set such that a first layer represents a sequence describing a query polynucleotide, each node represents a k-mer within a respective sequence, and each edge connects nodes representing identical or homologous k-mers, k being from 6 to 12;
searching said graph for continuous non-intersecting paths along edges of said graph; and
generating an output identifying a k-mer corresponding to at least one path as a nucleic acid sequence of functional interest.

23. The method according to claim 22, comprising, before said generating said output, iteratively repeating said constructing and said searching, each time for a shorter k-mer.

24. The method according to claim 23, comprising, at each iteration cycle, applying paths obtained in a previous iteration cycle as constraints for said search.

25. The method according to any of claims 22-24, wherein said searching comprises applying a path depth criterion as a constraint for said search, such that said search is preferential for deeper paths than for shallower paths.

26. The method according to any of claims 22-25, wherein said searching comprises applying an Integer Linear Program (ILP) to said graph.

27. The method according to any of claims 22-25, wherein said homologous polynucleotides are DNA sequences.

28. The method according to any of claims 22-25, wherein said homologous polynucleotides are RNA sequences.

29. The method according to any of claims 22-28, comprising aligning said sequences in said set according to a predetermined order, so as to provide a multiple alignment with multiple alignment layers, where a first layer is said query polynucleotide of said plurality of homologous polynucleotides, and wherein said multiple alignment layers respectively correspond to said layers of said graph.

30. The method of claim 29, wherein said predetermined order is evolution-dictated, optionally wherein said query is the most advanced in evolution is said homologous polynucleotides.

31. The method of any of claims 22-30, wherein a homology among said homologous k-mers is at least 70%.

32. The method of any one of claims 22-31, wherein said homologous polynucleotides comprise partial sequences.

33. The method of any one of claims 22-32, wherein said homologous polynucleotides are selected from the group consisting of 3′UTR, lncRNA and enhancer.
